SEQUENCE LISTING (28 sequences)

SEQ ID NO: 1 - DNA, 17 bp, Artificial Sequence
  Primer
aggaactgcaggtgtcc

SEQ ID NO: 2 - DNA, 20 bp, Artificial Sequence
  Primer
cagctacaggtgtccactcc

SEQ ID NO: 3 - DNA, 19 bp, Artificial Sequence
  Primer
tggcagcarcagctacagg

SEQ ID NO: 4 - DNA, 19 bp, Artificial Sequence
  Primer
ctgcctggtgacattccca

SEQ ID NO: 5 - DNA, 17 bp, Artificial Sequence
  Primer
ccaagctgtgtcctgtc

SEQ ID NO: 6 - DNA, 20 bp, Artificial Sequence
  Primer
ttttaaaaggtgtccagkgt

SEQ ID NO: 7 - DNA, 23 bp, Artificial Sequence
  Primer
cctgtcagtaactrcaggtgtcc

SEQ ID NO: 8 - DNA, 20 bp, Artificial Sequence
  Primer
ttttaaaaggggtccagtgt

SEQ ID NO: 9 - DNA, 19 bp, Artificial Sequence
  Primer
cgttcctggtatcctgtct

SEQ ID NO: 10 - DNA, 21 bp, Artificial Sequence
  Primer
atgaagttgtggytraactgg

SEQ ID NO: 11 - DNA, 18 bp, Artificial Sequence
  Primer
tgttggggctkaagtggg

SEQ ID NO: 12 - DNA, 23 bp, Artificial Sequence
  Primer
agaaggtgtgcacaccgctggac

SEQ ID NO: 13 - DNA, 31 bp, Artificial Sequence
  Primer
gggaattcgaggtgcagctgcaggagtctgg

SEQ ID NO: 14 - DNA, 22 bp, Artificial Sequence
  Primer
gctcagggaartagcccttgac

SEQ ID NO: 15 - DNA, 24 bp, Artificial Sequence
  Primer
rgtgcagattttcagcttcctgct

SEQ ID NO: 16 - DNA, 24 bp, Artificial Sequence
  Primer
tggacatgagggcycctgctcagt

SEQ ID NO: 17 - DNA, 24 bp, Artificial Sequence
  Primer
ctstggttgtctggtgttgaygga

SEQ ID NO: 18 - DNA, 22 bp, Artificial Sequence
  Primer
gttgctgctgctgtggcttaca

SEQ ID NO: 19 - DNA, 17 bp, Artificial Sequence
  Primer
gtatctggtacctgtgg

SEQ ID NO: 20 - DNA, 22 bp, Artificial Sequence
  Primer
tgcctgttaggctgttggtgct

SEQ ID NO: 21 - DNA, 25 bp, Artificial Sequence
  Primer
gctcagttccttggtctcctgttgc

SEQ ID NO: 22 - DNA, 20 bp, Artificial Sequence
  Primer
tgggtgctgctgctctgggt

SEQ ID NO: 23 - DNA, 27 bp, Artificial Sequence
  Primer
cagttcctgtttctgttartgctctgg

SEQ ID NO: 24 - DNA, 26 bp, Artificial Sequence
  Primer
tgctctggttatatggtgctgatggg

SEQ ID NO: 25 - DNA, 21 bp, Artificial Sequence
  Primer
actgaggcacctccagatgtt

SEQ ID NO: 26 - DNA, 24 bp, Artificial Sequence
  Primer
gayattgtgmtsacmcarwctmca

SEQ ID NO: 27 - DNA, 19 bp, Artificial Sequence
  Primer
tgggaagatggatacagtt

SEQ ID NO: 28 - protein, 10 aa, Artificial Sequence
  Cell adhesion peptide
RGDSPGERCG

(19) United States
(12) Patent Application Publication
(10) Pub. No.: US 2025/0290844 A1
(43) Pub. Date: Sep. 18, 2025

(54) METHODS OF USING SHAPED PARTICLES IN FLOW CYTOMETERS FOR ASSAYS ON B CELLS AND T CELLS

(71) Applicant: THE REGENTS OF THE UNIVERSITY OF CALIFORNIA, Oakland, CA (US)

(72) Inventors: Dino Di Carlo, Los Angeles, CA (US); Joseph de Rutte, Los Angeles, CA (US); Robert Dimatteo, Sherman Oaks, CA (US); Maani Meshkin Archang, Los Angeles, CA (US); Robert Damoiseaux, Beverly Hills, CA (US); Doyeon Koo, Los Angeles, CA (US)

(73) Assignee: THE REGENTS OF THE UNIVERSITY OF CALIFORNIA, Oakland, CA (US)

(21) Appl. No.: 18/560,103

(22) PCT Filed: May 5, 2022
(86) PCT No.: PCT/US2022/027924

§ 371 Date: Nov. 10, 2023

Related U.S. Application Data
(60) Provisional application No. 63/186,719, filed on May 10, 2021.

(51) Int. Cl. G01N 15/149; G01N 15/10; G01N 33/543; G01N 33/68

(52) U.S. Cl. CPC G01N 15/149; G01N 33/543; G01N 33/68; G01N 2015/1006; G01N 2333/78

(57) ABSTRACT

Suspendable shaped particles or nanovials are used to viably sort single cells based on their secreted product at high-throughput using only commonly accessible lab infrastructure. These shaped particles act as a solid support which facilitates cell attachment, templates formation of uniform aqueous compartments which prevent cross-talk between cells, and captures secreted proteins. Using this platform, high-throughput screening of producer cells on relative IgG production, B/plasma cells based on secreted antibody binding to antigen, and T cells based on cytokine secretion is demonstrated using commercially available flow sorters. These shaped particles are easily distributed and used, democratizing access to high-throughput functional cell screening.

### RELATED APPLICATION

This Application is a U.S. National Stage filing under 35 U.S.C. § 371 of International Application No. PCT/US2022/027924, filed on May 5, 2022, which claims priority to U.S. Provisional Patent Application No. 63/186,719 filed on May 10, 2021, which are hereby incorporated by reference in its entirety. Priority is claimed pursuant to 35 U.S.C. § 119, 371 and any other applicable statute.

### STATEMENT REGARDING FEDERALLY SPONSORED RESEARCH AND DEVELOPMENT

This invention was made with government support under Grant Number GM126414, awarded by National Institutes of Health. The government has certain rights in the invention.

### TECHNICAL FIELD

The technical field generally relates to methods of using shaped particles or nanovials for massively parallel single-cell functional analysis and sorting.

### BACKGROUND

The microwell plate format has become a foundation for biological assays because of the ability to scale experiments and integrate with lab automation infrastructure. This simple piece of plasticware allows scientists to add or exchange reagents while preventing cross-talk between samples. The bottom surface of each well can be functionalized to adhere cells, promote cell growth, and perform biomolecular reactions that allow for colorimetric and fluorescent readouts. Biological samples of interest can then be isolated by pipetting fluid from a well. Despite its simplicity and utility, even the smallest, 1536-well format, plates hold volumes that are hundreds of thousands of times larger than cells in each well limiting the ability to study the functional properties, such as secreted products, from single cells.

The ability to perform functional biological assays at the resolution of individual cells promises to deepen the understanding of biology and accelerate the development of new biological products. For example, sorting of rare B cells or plasma cells directly based on activity of high affinity secreted antibodies allows for the acquisition of gene sequences that can be used to make new antibody drugs or diagnostic affinity reagents. Selection of stable production-grade cells, such as Chinese hamster ovary (CHO), based on their production rate of humanized IgG protein would allow for the selection of specific clones useful for monoclonal antibody production environments.

Although technologies have emerged to enable to the probing of single cells, there are significant tradeoffs that either limit functionality or inhibit widespread adoption. Fluorescence activated cell sorting is one of the first single-cell technologies to gain wide spread adoption, enabling users to probe and sort individual cells based on light scatter properties and fluorescently stained molecules at throughputs over 10,000 events per second. Screening of viable cells is typically limited to non-functional parameters such as exterior cell surface proteins (i.e., the clusters of differentiation markers, or CDs) while intracellular markers can be analyzed but require membrane permeabilization and fixation. Approaches have been developed to capture secreted products directly to the cell surface, but these techniques suffer from significant crosstalk between cells. Microfluidic technologies have emerged that enable the creation of volumes approaching the size of cells and high-throughput analysis of single cells based on additional properties such as secreted molecules. Devices with microscale chambers as well as water-in-oil droplets generated using microfluidic devices have been employed to isolate cells, accumulate secreted product, and even sort. Despite the utility of these existing platforms, the ability to add reagents and wash reactions is limited, and microfluidically-generated droplets lack the solid surface of a microwell plate which is critical for standard assay formats like sandwich immunoassays or enzyme-linked immunosorbent assays (ELISA). Approaches to overcome these shortcomings have been developed, but each requires specialized assay formats, and often extremely expensive equipment to perform the assays, which hinders widespread adoption.

### SUMMARY

A new approach is disclosed herein to perform functional assays on individual cells at high-throughput using structured or shaped particles, which act as suspendable and sortable microwells. These shaped particles or “nanovials” hold single cells in sub-nanoliter volumes of fluid, 100,000 times less volume then 1536-format microwells yet require no new instrumentation. Fluids are easily exchanged by centrifugation and pipetting, and each compartment can be sealed and unsealed using biocompatible oils to prevent cross-talk between samples. The surfaces can be modified to bind cells or capture biomolecules for molecular readout. The shaped particles or nanovials can be analyzed and isolated using commonly available FACS instruments enabling screening at rates of >1000 events per second. While other particle systems have been utilized to hold sub-nanoliter volumes, the approach disclosed herein is the first approach that allows attaching and protection of cells in cavities within particles and unlocks new capabilities to expand scale of analysis by integration with flow cytometry that was not previously achieved. Using this new particle-based format, a screen of >500,000 events in less than an hour was conducted using commercial flow cytometers and FACS instruments, demonstrating single-cell antibody discovery and cell line development workflows that can identify rare antibody secreting cells from an order of magnitude more cells than previous approaches all within one day. A similar experiment would require >1300 384-well plates and at least a week of additional culture to grow up a sufficiently large clonal population of cells to detect secretions in the larger volumes. Other applications may include the selection of producer cell(s) with either optimized yields or stability of production. The strength of this system is that it is widely usable.

In one embodiment, a shaped particle system includes a plurality of three-dimensional shaped particles (i.e., nanovials), each shaped particle having a void or cavity formed therein that is a single opening to an external environment of the shaped particle, wherein each shaped particle further includes a poly-L-lysine (PLL)-containing surface in the void or cavity. An extracellular matrix protein or protein fragment may also be disposed on the surface of the void or cavity.

In another embodiment, a shaped particle system includes a plurality of three-dimensional shaped particles, each shaped particle having a void or cavity formed therein that includes a single opening to an external environment of the shaped particles, wherein each shaped particle further comprises a cell binding moiety and one or more capture agents disposed on a surface of the shaped particle in the void or cavity. For example, the antibodies to the cell surface marker may include one or more of: (1) anti-CD45 antibodies or a fragment thereof, anti-CD19 antibodies or a fragment thereof, anti-CD3 antibodies or a fragment thereof, anti-CD28 antibodies or a fragment thereof, streptavidin, biotin, an antigen, a peptide-major histocompatibility complex and the one or more capture agents include one or more of: (2) anti-IL-2, anti-TNF-α, anti-IFN-γ antibodies or fragments thereof.

In another embodiment, a method of screening B or T cells for one or more secretions of interest using a plurality of three-dimensional shaped particles includes: loading single B or T cells into respective voids or cavities formed in the plurality of three-dimensional shaped particles; capturing the one or more secretions of interest with one or more capture agents disposed on or in the three-dimensional shaped particles; labeling the one or more captured secretions of interest with fluorescent reporter(s); sorting the three-dimensional shaped particles using a flow cytometer or fluorescence activated cell sorter based on a fluorescence signal from the fluorescent reporter(s) to create a sorted population of three-dimensional shaped particles. Nucleic acid sequence analysis may then be performed on nucleic acids contained in at least some of the sorted population of three-dimensional shaped particles.

In another embodiment, a method of sorting shaped particles loaded with cells using a flow cytometer or FACS instrument includes: providing a population of shaped particles loaded with single cells into respective voids or cavities formed therein; capturing a secretion from the cells on one or more of the shaped particle containing single cells therein; exposing the one or more shaped particles with the captured secretion to a fluorescent reporter; flowing the population of shaped particles loaded with single cells through the flow cytometer or FACS instrument; optically interrogating the shaped particles in the flow cytometer or FACS instrument to measure a fluorescence signal for each shaped particle that comprises two or more of: a fluorescence peak area, fluorescence peak height, and fluorescence peak width; and identifying and sorting a sub-population of the shaped particles based at least in part on a threshold or gate on two or more of: fluorescence peak area, fluorescence peak height, and fluorescence peak width. In some embodiments, the sub-population is identified and sorted based on a ratio between fluorescence peak area and fluorescence peak height. For example, the sub-population may be identified and sorted based on a threshold ratio, wherein a sub-population having a ratio above the threshold ratio are sorted separately from another sub-population having a ratio below the threshold ratio. This may include, for example, certain phenotypes or populations of B cells or T cells.

### BRIEF DESCRIPTION OF THE DRAWINGS

FIG. 1A schematically illustrates a shaped particle or nanovial that contains a cell therein.

FIGS. 2A-2D illustrates a microparticle platform for high-throughput single cells secretion screening. FIG. 2A: individual cells are loaded into prefabricated shaped particles or nanovial containers (nanovials) and bound using a variety of binding schemes. Particles and associated cells are agitated by pipetting with biocompatible oil and surfactant to generate monodisperse compartments dictated by the particle size (dropicles) preventing crosstalk between microcontainers. Cells are incubated to accumulate secretions on associated particles and transferred back to a water phase for fluorescent labeling. Particles, cells, and associated secretions are then analyzed and sorted using high-throughput commercial flow sorters. Isolated populations can then be analyzed downstream for different phenotypic (growth, productivity) and genotypic properties (RNA expression, nucleic acid sequencing) and surface marker patterns which allow for subgrouping of resulting cells. FIG. 2B: photograph of a tube with 2 million dropicles formed using simple pipetting steps in less than 1 minute (left). Fluorescence microscopy image of uniform dropicles formed with fluorescently stained nanovials and water-soluble dye (right) show distinctly sealed sub-nanoliter volumes. FIG. 2C: microscopy image of nanovials with cells and associated secretions after unsealing the nanovials and staining with fluorescent labels. Scale bar is 100 μm. FIG. 2D is an example of a flow cytometry plot and post-sort images of enriched highly-secreting populations. Scale bars are 100 μm.

FIGS. 3A-3D: Loading and binding of single cells into nanovials. FIG. 3A: particles are loaded into wells and settle with their cavities oriented upright due to their asymmetric center of mass. Cells are then seeded into the open cavities and attached using various binding moieties. Unbound cells are washed away using a reversible cell strainer and recovered particles and associated cells are analyzed. FIG. 3B: example microscopy images of fluorescently tagged cells bound to nanovials. FIG. 3C: comparison of cell binding is plotted below for different cell types and binding moieties. Adherent producer cells such as CHO DP12 (Blue) and HEK293 cells (magenta) bound to particle cavities through integrin binding sites (RGD peptide) linked to the particle matrix. Cell retention for suspension adapted ExpiCHO (green) was increased by introduction of positively charged poly-L-lysine (PLL). Inclusion of surface marker specific antibodies on the nanovial surface increased retention of B-Cells (red). FIG. 3D: loading of cells into nanovial cavities follows Poisson statistics. Error bars represent SD of n=3 samples for loading distribution.

FIGS. 4A-4D: Massively parallel device-free formation of uniform droplets. FIG. 4A: biocompatible oil and surfactant are added to a tube containing concentrated particles. The suspension is pipetted vigorously for 1 minute to generate smaller and smaller emulsions. FIG. 4B: microscopy images of the emulsion show a uniform population of droplets containing particles and smaller background satellite droplets. FIG. 4C: histograms of dropicle diameter for different nanovial sizes. Nanovial containing droplets are highly-uniform and comparable to advanced microfluidic techniques (CV<5%). FIG. 4D: For both all nanovial sizes nearly all droplets formed have either 0 or 1 particle encapsulated, with only a small fraction (<1%) containing 2 or more particles per droplet. Scale bars, 200 μm.

FIGS. 5A-5F: Analysis of single-cell secretions using dropicles. FIGS. 5A-5B: characterization of cross-talk when performing a secretion assay with human IgG producing CHO cells with and without an encapsulation step. FIG. 5A: significant crosstalk is observed when cells and particles are left in an aqueous phase for >10 hours during secretion incubation. FIG. 5B: minimal cross-talk is observed when cells and associated particles are encapsulated in oil during the secretion incubation step. Thresholds in both (FIG. 5A) and (FIG. 5B) are set at 3 standard deviations above empty particle signal for the encapsulation condition. FIG. 5C: as the secretion incubation time is increased, the distribution in measured secretions increases as expected. FIG. 5D: averaged secretion measurements across the producing population shows signal increasing proportionally with time across 3 separate samples. FIG. 5E: a secretion assay was performed on a mixture of human IgG producing CHO cells (magenta) spiked into a non-producing CHO cell population (blue). Samples were sorted based on positive IgG signal using FACS and imaged using fluorescence microscopy to determine purity and enrichment ratio. FIG. 5F: spiked target cells were successfully isolated across a range of dilutions (1:5-1:1000) at purity up to 99% and nearly 1000-fold enrichment. 100,000 single cells were sorted during the enrichment study.

FIGS. 6A-6D: Selection of high secretion cell sub-populations using FACS. FIG. 6A: the secreting population of cells were gated and sorted based on IgG secretion signal and CellTracker (CT) labelling. Both cells with any secretion signal disparate from background as well as the top 20% of secretors were separately sorted. FIG. 6B: microscopy images before and after sorting show enrichment of secreting cells with fluorescence intensity proportional to the selection criteria. FIG. 6C: example images of cells expanded out of nanovials after encapsulation and release. FIG. 6D: after expanding isolated cells for 10 days, bulk ELISA was performed to determine the production rate for the different sub-populations. Full experiment was performed with separate cell passages (n=4). Error bars represent s.d.

FIGS. 7A-7E. Detection and sorting of antigen-specific antibody secreting cells. FIG. 7A: hybridomas or B cells loaded into nanovials secrete antibodies which are captured onto the nanovial surface via biotinylated capture antibodies. Captured IgG is then labeled with fluorescent antigen to assess specificity of the secreted IgG. FIG. 7B: fluorescence microscopy images reveal specific signal from binding to Alexa Fluor™ 647 conjugated when egg lysozyme (HEL-647) on nanovials containing a hybridoma line (HyHEL-5) secreting IgG HEL while a cell line (9E10) secreting an off-target IgG results in no signal. FIG. 7C: HyHel-5 hybridomas spiked into 9E10 hybridomas (CellTracker™ Blue) at a 1:25 ratio were assayed by fluorescence microscopy for HEL-specific signal before FACS sorting (Pre-Sort) and after sorting using a high HEL-AF647 gate (Post-Sort). A table showing the Pre-Sort and Post-Sort statistics is shown. FIG. 7D: Using the index sorting function of the flow cytometer single hybridomas loaded onto nanovials were sorted and (i) demonstrate that RNA can be reverse transcribed and amplified from single cells loaded on nanovials at comparable rates to single cells sorted that were freely suspended. A gel band corresponding to the correct heavy chain amplicon length is observed. (ii) individual hybridomas can be expanded into a clonal colony directly from the nanovials following sorting. FIG. 7E: antibody-secreting B cells from ovalbumin (OVA)-immunized mice are assayed for antigen specificity using nanovials. OVA-specific signal is observed directly on the nanovials by fluorescence microscopy down to 20,000-times dilution of serum from OVA-immunized mice but not control mice. B cells loaded on nanovials were observed to have both cell surface signal and OVA-specific signal by fluorescence microscopy. Following FACS sorting based on a gate encompassing the top 2.17% of OVA fluorescence area signal, OVA-specific antibody-secreting cells were successfully imaged possessing minimal cell surface staining with OVA, along with some cells staining strongly but possessing no OVA-specific secreted antibody signal.

FIGS. 8A-8E: Analysis of single-cell IgG secretion distributions for transiently transfected cells on nanovials. FIG. 8A: biotinylated HEK293 cells stained with CellTracker™ Violet were loaded onto streptavidin coated nanovials. After cell binding, biotinylated anti-IgG was bound to remaining streptavidin sites. Cells are then transiently transfected with an IgG expression vector and incubated for 16 hrs. Cells and nanovials were then emulsified in oil and incubated for an addition 32 hrs to accumulate secretion signal. Cells and nanovials were transferred back to buffer solution and stained with fluorescent secondary antibodies against human IgG and analyzed using flow cytometry. FIG. 8B: representative forward and side scatter gating strategy is shown. Debris and residual oil are removed by excluding low scatter signal. FIG. 8C: to accurately differentiate subpopulations, cells were tagged and gated based on CellTracker™ Violet signal and nanovials were tagged and gated based on AlexaFluor™ 647 signal. FIG. 8D: a fraction of cell-containing nanovials were observed to have IgG signal above empty nanovials indicating cell secretion-specific signal. FIG. 8E: for both constructs a signal above a negative DNA control sample is seen indicating cell secretion-specific signal.

FIG. 9 illustrates the schematic of two fluorescence-linked immunosorbent assay setups for antigen specific B/plasma cell detection, quantification, and sorting.

FIG. 10 illustrates the dynamic range and limit of detection assay with secreted antibody capture on nanovials. Upper left: the fluorescent-linked immunosorbent assay (FLISA) schematic for capture and detection of antigen specific antibodies in the dynamic range assay. Capture agents on the nanovials were ovalbumin (OVA) and fluorescent reporters were an anti-IgG antibody. Top right: histograms of intensity and mean intensity of nanovials as a function of log(1/dilution) is shown for sera from OVA vaccinated mice and diluted by dilution-fold with buffer. Lower left: control. Bottom right: histograms of intensity and mean intensity of nanovials as a function of log(1/dilution) is shown for sera from non-vaccinated mice as a control.

FIGS. 11A-11B: Assessment of the dynamic range of capture and detection of antigen specific antibodies on nanovials. FIG. 11A: schematic shows the assay format in which biotinylated anti-Mouse IgG H+L antibody acts as a capture agent on nanovials to bind to secreted antibodies. Secreted antibodies are detected with a fluorescent reporter, fluorescently labeled OVA antigen. Images are shown of nanovials incubated with serial dilutions of sera from OVA vaccinated and non-vaccinated mice and stained with fluorescently conjugated ovalbumin. Antigen-specific antibodies in mice sera at dilutions down to 1:20,000 are detected and show no non-specific binding for control sera by fluorescence microscopy. FIG. 11B: histograms of intensity and mean intensity of nanovials as a function of log(1/dilution) is shown for sera from non-vaccinated mice as a control.

FIGS. 12A-12C: Assessment of components of the workflow for detection and sorting of antigen specific antibody secreting cells. FIG. 12A: fluorescence and brightfield micrographs of nanovials seeded with cells before and after removal of excess cells and sorting for cell containing nanovials. FIG. 12B: fluorescence and brightfield micrographs of B/plasma cells after a complete workflow for detection of antigen specific antibody secretion signal via capture of secreted antibodies on nanovials using anti-IgG and staining with fluorescently-labeled antigen. Fluorescently-labeled antigen (OVA) stains nanovials from cells secreting antibodies that bind OVA and in some cases cells. Nanovials shown were sorted by FACS (Sony SH800) using a gate H for high Cy5-fluorescence area and high Cy5-fluorescence height. FIG. 12C: flow cytometry plots of the B/plasma cell-containing nanovials after staining with fluorescently-labeled antigen (OVA). Higher fluorescence (Cy5) area for a specific fluorescence (Cy5) height, or higher fluorescence (Cy5) width can be used to identify nanovials with bound secreted antibody across their surface specific to antigen compared to cell surface staining which results in lower fluorescence area for the same fluorescence height or lower fluorescence width for the same fluorescence height.

FIGS. 13A-13D illustrate gel results from RT-PCR of single hybridoma cells freely suspended and loaded on nanovials following FACS sorting into separate wells of a 96 well plate. FIG. 13A-B: RT-PCR results from hybridoma cells off of nanovials. Bands showing the correct DNA base pair length (shown on the left) for amplicons of the heavy chain are observed for a fraction of the wells (solid boxes). FIG. 13C-D RT-PCR results from hybridoma cells loaded on nanovials. Bands showing the correct DNA base pair length (shown on the left) for amplicons of the heavy chain are observed for a fraction of the wells (solid boxes).

FIGS. 14A-14H: Nanovial fabrication using an aqueous two-phase system combined with droplet microfluidics. FIG. 14A: a solution comprised of UV reactive PEG and photoinitiator is co-flowed with a solution containing dextran, dithiol crosslinkers, and RGD peptides in a microfluidic droplet generator. FIG. 14B: a third solution of oil and surfactant is injected into the device to generate water-in-oil droplets at a rate of ˜1000 Hz. FIG. 14C: downstream on the device PEG and dextran undergo phase separation resulting in two distinct regions in the droplet. The droplets are exposed with UV light at the end of the device to crosslink the PEG rich portion of the droplet, while the dextran rich region remains as a liquid. FIG. 14D: the resulting shaped particles or nanovials are then collected, washed to remove oil and dextran, and stored for later use. FIG. 14E: photograph of a 15 mL conical tube with 10 million drop-carrier particles fabricated in ˜3 hours. FIG. 14F: nanovials fabricated using this approach are highly monodisperse with outer diameter CV of 1.5% and cavity opening diameter CV of 2.1% (n=409). Particle uniformity was calculated by analyzing fluorescence microscopy images. FIG. 14G: fluorescence microscopy image of biotinylated nanovials stained with Alexa Fluor 568 Streptavidin (AF568-SA). A large fraction of the seeded particles settles with their cavities exposed upright. FIG. 14H: nanovial cavity morphology and upright orientation was confirmed using confocal microscopy.

FIGS. 15A-15B: Nanovial morphology can be tuned by adjusting the concentrations of PEG and dextran in the droplets. FIG. 15A: the morphology of nanovials is dictated by a balance of interfacial tensions (γ) between the different phases. γ_{PEG/Oil} and γ_{DEX/oil} are expected to vary minimally, the balance of interfacial tensions and the resulting morphology is expected to be dictated mostly by a change in γ_{PEG/DEX}. FIG. 15B: experimentally determined phase diagram of 4-Arm PEG Norbornene, and dextran. At very low concentrations phase separation does not occur resulting in a spherical particle (i). As the total concentration of PEG and dextran is increased above the binodal line, phase separation occurs enabling fabrication of cavity-containing shaped particles or nanovials. (I) The relative opening diameter of the particle cavity is increased by increasing the total polymer concentration and thus γ_{PEG/DEX} resulting in relative opening size of 48-63% of the outer diameter. (II) By adjusting the concentration ratio of dextran and PEG, particles can be fabricated with different relative cavity sizes. Here the relative cavity diameter is from 34-76% the outer diameter. Particles fabricated using a PEG concentration of 13% and dextran concentration of 5% (denoted by *) were found to have high structural integrity while maintaining a relatively large cavity opening to enable efficient cell loading. This condition was used for all other experiments in this work.

FIG. 16: Compatibility of a range of nanovial sizes with different cell types and instruments. Nanovials with high uniformity were fabricated with mean outer diameters ranging from 35 to 105 μm and respective mean cavity diameters of 20 to 62 μm. Smaller nanovials are more compatible with standard flow cytometers which are more accessible and allow higher throughputs, but are limited to smaller cell types. Larger nanovials are compatible with larger cells and cell colonies/clusters, but have fewer commercially available flow sorters. Notably all sizes are compatible with microscopy imaging, pipettes, liquid handlers, and analysis in microfluidic devices and channels with channel dimensions greater than the maximum nanovial dimensions. Scale=100 μm.

FIG. 17: Time-lapse images of nanovials seeded into a well plate. To prepare nanovials for cell seeding they are pipetted into a well plate by dispensing liquid up and down in circular motions. Due to the unique morphology of the nanovials they tend to settle with their cavities oriented upright and displace adjacent nanovials aside to form a monolayer.

FIGS. 18A-18B: Representative sorting gates for nanovials on different FACS instruments. FIG. 18A: biosorter from Union Biometrica. FIG. 18B: on-Chip Sort from On-Chip Biotechnologies.

FIGS. 19A-19B: Energy minimalization theory supports monodisperse dropicle formation. FIG. 19A: simulated volume-energy (V-E) curves show that droplets formed with nanovials (dropicles) have a minimal energy configuration when the size of the droplet surrounding it approaches the size of the nanovial. Here the system energy and volume are normalized by the minimum energy values (E0 and V0, respectively). A free droplet of the same outer diameter follows along the same energy curve until the minimal energy point. As energy is added to the system (e.g., by shearing with a pipette) excess fluid is shed from the nanovial (iii), until the minimal energy volume is reached (ii). Lower volumes could only be achieved by drying out excess fluid via evaporation (i). FIG. 19B: on the basis of the V-E curve in (FIG. 19A), splitting of a drop containing two nanovials is theoretically expected to depend on the total fluid volume. At volumes below twice V0 nanovials are expected to remain as aggregates. At slightly larger volumes if enough energy is added volume is expected to split evenly based on energy minimalization theory. At larger volumes the fluid is expected to distribute asymmetrically between nanovials; one with a smaller volume approaching V0 and one with the remainder of the fluid. The nanovial with the larger volume may undergo additional volume reduction via shearing until reaching V0.

FIGS. 20A-20E: Characterization of cell viability and growth after dropicle formation and release. FIG. 20A: brightfield and fluorescence microscopy image of cells encapsulated in dropicles. Biotinylated nanovials are stained with Alexa Fluor 568 streptavidin and cells are stained with calcein AM. FIG. 20B: cells maintained high viability after dropicle formation and release. Viability was assessed by staining with calcein AM and propidium iodide after recovering particles and cells from droplets. Live/dead assay was performed with a minimum of n=3 samples, cell number >1000 per condition. FIG. 20C: Cells initially remain in the particle cavities after releasing them. Colonies derived from single cells remain in the particle cavities during initial expansion. FIG. 20D: after significant accumulation of cells in the cavities, cells begin to spread to the outside of the cavities and onto the well plate surface. FIG. 20E: cell growth in nanovials post dropicle release is comparable to cell growth on standard well plates (control) (n=3). Growth is characterized by fold change in cell number over a 24 hr period as characterized by counting cell number using CellTracker staining.

FIG. 21: Detailed overview of high IgG producer enrichment workflow. RGD coated nanovials are first seeded into a well plate and settle with cavities exposed upright. CHO cells are then seeded into the exposed cavities and incubated to allow attachment via integrin binding. Nanovials and attached cells are recovered by pipetting and transferred into a centrifuge or conical tube. Nanovials are then modified sequentially with streptavidin and biotinylated anti-IgG antibodies. The nanovials are then emulsified to prevent crosstalk and incubated in a CO₂ incubator and accumulated secreted antibodies from cells. Nanovials, cells, and associated secretions are transferred back into water phase, labeled with fluorescent reporter molecules (anti-human IgG antibodies with a fluorescent label) and analyzed with flow to measure amount of secreted product. The higher producing cells, as measured by fluorescence are sorted out and expanded in a well plate for downstream productivity assessment. The full secretion assay workflow is completed in a single day and cells are expanded for a week after enrichment for downstream assessment via bulk well plate ELISA.

FIG. 22: Detailed overview of workflow for selection of antigen specific antibody secreting cells and linked repertoire sequencing. Antibody-conjugated nanovials (e.g., containing anti-CD45 and anti-IgG or anti-Fc) are seeded into a well plate and settle with cavities exposed upright. B cells, plasma blasts, and/or plasma cells are then seeded into the exposed cavities and incubated to allow binding. Nanovials and attached cells are transferred to a microcentrifuge tube by pipetting. Any background antibodies are washed away and the nanovials and cells are then optionally emulsified to prevent crosstalk and incubated to accumulate secreted antibodies on the surface of the nanovials. If emulsified, the nanovials, cells, and captured secreted antibodies are then transferred back into water phase and stained with fluorescent reporter molecules (e.g., fluorescently-labeled antigen). Using FACS, nanovials are screened in high-throughput and single nanovials with a cell secreting antibody with affinity to the antigen of interest are index sorted into a 96-well plate based on fluorescence signal on the nanovial. Lysis and downstream sequencing of nucleic acids from the single sorted cells are performed. Sequencing of paired heavy and light chain sequences is shown. Information from the sequencing is linked to the information on fluorescence signal for the sorted nanovial to create a linked data set of antigen binding/affinity and paired heavy and light chain sequences.

FIGS. 23A-23C: Optimization of T cell adhesion to nanovial surfaces. FIG. 23A: cells were loaded into nanovials either without modification or after modifying the nanovial surface with anti-CD45 antibodies. Samples were incubated for 1 hour and then strained through a 20 μm cell strainer to remove unbound cells. Recovered samples were imaged and analyzed for the fraction of particles containing cells. FIG. 23B: after cell analysis it was found that ˜8.6% of CD45 modified particles retained a cell after straining and recovery, in comparison less than 0.01% of unmodified particles contained a cell. FIG. 23C: particle loading was increased by increasing the concentration of antibodies on particles and resuspending particles and cells after the suspension had settled in the well plate. 5.5 μg/mL of anti-CD45 antibodies were utilized for all further studies of T cells as this was the lowest concentration of antibody used that gave robust cell loading. Scale bars are 50 μm in width.

FIGS. 24A-24B: Recombinant cytokines were used to verify the limit of detection and dynamic range for on-nanovial cytokine secretion assays. Three separate experiments were run to assess the potential to measure IL-2, TNF-α, and IFN-γ on nanovials using both (FIG. 24A) flow cytometry and (FIG. 24B) fluorescence microscopy. The limit of detection for IL-2 and TNF-α was 10 ng/mL using flow cytometry, and 100 ng/mL through fluorescence microscopy. The limit of detection of IFN-γ was 1 ng/mL using flow cytometry and 10 ng/mL using fluorescence microscopy. Scale bars are 50 μm in width.

FIGS. 25A-25C: Nanovials were used to measure TNF-α production from (FIG. 25A) conditioned media, (FIG. 25B) co-incubation with cells in a microwell plate, and (FIG. 25C) individual cells encapsulated with particles within picoliter volume droplets (dropicles). All three samples yielded positive fluorescence signal. Conditioned media samples gave uniform fluorescence signal across all particles. Samples that were co-incubated with cells yielded localized fluorescent signals due to neighboring secreting cells. Emulsified samples containing cells gave bright signals within the particle cavities. Events of particular interest are delineated with a green outline and magnified below the image. Scale bars are 50 μm in width.

FIGS. 26A-26B: Due to the relatively low rate of cytokine production from single T cells, single cell secretion readouts can be obtained without emulsification with limited cross-talk. Here cells were seeded into nanovials allowed to attach for one hour and background cells were strained out of the sample. After recovering from straining, samples were activated with PMA and ionomycin and incubated in a single 24 well plate for 3 hours. After recovery, subsequent labeling with fluorescent reporters for TNF-α or IFN-γ was conducted. Particles containing individual cells yielded specific fluorescence within the nanovial cavity associated with secretion of TNF-α (FIG. 26A) or IFN-γ (FIG. 26B). Fluorescence microscopy images are shown before FACS sorting of nanovials containing cells and after sorting nanovials containing cells and with fluorescence height and area signal above a threshold value. Insets are zoomed in images for specific nanovials in brightfield and fluorescence channels. A combination of fluorescence height, area, and/or width can be used to further detect and/or sort secreted cytokines bound to nanovials. Wider/larger area for a specific fluorescence height corresponds to stained nanovials, which have a larger width than cells. Nanovial maximum diameter is ˜50 micrometer.

FIG. 27 illustrates an overview of high-throughput analysis and sorting of individual T cells based on secreted cytokines using nanovial technology. Operation A: Nanovials are functionalized with biotinylated cytokine capture antibodies and a cell binding motif via streptavidin-biotin chemistry. Operation B: T cells are loaded into the cavities of nanovials in a well plate and any unbound cells are removed using a cell strainer. Scale bars represent 25 μm. Operation C: T cells are activated for 3 hours and secreted cytokines are captured in the cavity of the nanovial. Operation D: Captured cytokines are labeled with a fluorescent detection antibody and cells may be labeled with additional surface markers. Operation E: Cytokine binding to nanovials can be distinguished from non-specific cell staining using a combination of fluorescence peak area and height signals associated with staining of the larger nanovials. Cells are sorted based on the secretion signal as well as cell surface markers using a commercial FACS. Scale bars represent 100 μm.

FIGS. 28A-28B: Nanovial fabrication and functionalization with multiple antibodies. FIG. 28A: An aqueous phase comprised of 4-arm PEG acrylate and photoinitiator is co-flowed with a gelatin solution in a microfluidic droplet generator to make uniform monodisperse aqueous two-phase water-in-oil droplets. After droplet formation, PEG and gelatin undergo phase separation and then are exposed to UV light at the end of the device to cross-link the 4-arm PEG acrylate. After collection, any excess polymerized PEG, gelatin, or oil are removed during washing and cross-linked nanovials are incubated with Sulfo-NHS-biotin to be biotinylated. Scale bars represent 50 m. FIG. 28B: Flow cytometry fluorescence histograms of nanovials following the cytokine capture assay with different capture antibody concentrations and recombinant cytokines. Nanovials were functionalized with cell binding motif (anti-CD45) and two cytokine capture antibodies at different concentrations (anti-IFN-γ, anti-TNF-α) and were incubated with recombinant TNF-α or IFN-γ. The ability of nanovials to detect each individual cytokine was not significantly affected with the presence of other cytokine capture antibody. There was also a capture antibody concentration-dependent shift in the average intensity on the nanovials.

FIGS. 29A-29C: Loading statistics and dynamic range of nanovials. FIG. 29A: T cells were loaded onto anti-CD45 and anti-IFN-γ labeled nanovials at different cell number to nanovial ratios. The highest single-cell loading efficiency was achieved when cells were seeded at 1.6 cells to nanovials. Increased cell seeding density resulted in a larger fraction of nanovials with two or more T cells inside the cavities of the nanovial. Loading of cells into nanovial cavities followed Poisson statistics. FIG. 29B: Top: Comparison of cell binding motifs (anti-CD45, anti-CD3, anti-CD3/CD28. Nanovials conjugated with anti-CD45 showed the highest loading efficiency when cells were loaded after initial CD3/CD28 activation in culture. Bottom: Increased anti-CD45 concentration on nanovials increased cell binding by nearly 6-fold. FIG. 29C: Flow cytometry analysis and microscopy images showed that nanovials were able to detect each recombinant cytokine (anti-IFN-γ, anti-TNF-α, anti-IL-2) as low as 1 to 10 ng/mL above background with at least three orders of magnitude dynamic range. Each scale bar represents 50 μm.

FIGS. 30A-30B: Analysis and sorting of single cells based on cytokine secretion using FACS. FIG. 30A: T cells stimulated with PMA/ionomycin were allowed to secrete for 3 hours on nanovials and analyzed using a Sony SH800. Fluorescence peak area and height are shown where two different slope regions are used to differentiate spatially-distributed IFN-γ secretion signals on nanovials (low secretion and high secretion) from more punctate signal solely from non-specific cell surface binding (label binding to cell). Nanovials and loaded cells from these regions were sorted and confirmed to have different distribution of fluorescence signal, as shown in the images. Scale bars represent 50 μm. FIG. 30B: Nanovials with each cytokine (TNF-α or IL-2) secretion signal were sorted using area vs. height metrics. The ability to isolate on-nanovial cytokine staining was consistent across different cytokines as shown in fluorescence microscopy images. Scale bars represent 50 μm.

FIGS. 31A-31C: Using the fluorescence peak area and height to measure single-cell secretions on nanovials. FIG. 31A: Overview schematic of the nanovial fluorescence peak shape obtained when a nanovial transits a laser spot in a flow cytometer. A nanovial passes through the laser beam and generates scattered light and fluorescence signals in a time-dependent manner. The nanovial is fully illuminated and produces a maximum amount of fluorescence signal (Peak Height) when it is located at the center of the laser and as it flows out the signal drops back to the baseline. The area is the integral of the fluorescence intensity emitted over the entire transit event through the laser spot. FIG. 31B: Fluorescence microscopy images of pre-sort nanovials with cells showed two distinct fluorescence patterns, with dotted lines in insets outlining the nanovial boundaries: fluorescence spread across the nanovial cavity from secreted cytokines or fluorescence associated with cells on nanovials without signal across the cavity area. The fluorescence intensity profile was computed across the cavity of each image and the maximum intensity (height, H), area under the intensity curve (area, A), and the ratio between the area and height were calculated. Nanovials with spatially spread secretion signal and nanovials with label bound to cells had similar peak height values, but secretion signal had much higher area over height ratio (A/H). FIG. 31C: Flow cytometry histograms for calcein AM staining of cells on nanovials. Gates for high calcein AM are used to select living, intact cells, representing ˜20% of events. Scale bars represent 100 μm.

FIGS. 32A-32C: Selection of viable T cells based on cytokine secretion level. FIG. 32A: Captured cytokines and loaded T cells were labeled with fluorescently labeled detection antibodies (anti-IFN-γ BV421 or anti-TNF-α APC) and calcein AM live cell dye. Using a Sony SH800, the nanovial population was first gated for high calcein signal (as seen in FIG. 31C) and fluorescence secretion signal was quantified using the fluorescence peak area vs. fluorescence peak height (A/H). Nanovials with non-specific cell surface labeling (low A/H ratio) using a live cell gate such that only a single A/H line is observed. Single cells were sorted as high, medium and low secretors based on their IFN-γ and TNF-α secretion level. Microscopy images show each corresponding population, with dotted lines in insets outlining the nanovial boundaries. Scale bars represent 50 μm. FIG. 32B: Recovery and regrowth of cells in nanovials post-sort is seen in microscopy images. Cells increased in number after 7 days, forming in clusters. FIG. 32C: Two nanovial types were introduced together to evaluate cross-talk. Fluorescently labeled nanovials (AlexaFluor 488) without cells were mixed will T cell-loaded nanovials activated with PMA/ionomycin at a ratio of 1:2. Secretion signal was labeled and analyzed on both nanovial types by gating on the green fluorescence signal on nanovials (Alexa 488 Area). While cell-containing nanovials had significant IFN-γ secretion signal, less than 1% of nanovials without cells had signal in the gate, most at low fluorescence values. Scale bars represent 100 μm.

FIGS. 33A-33B: Analysis and sorting of single cells based on multiple secreted cytokines. FIG. 33A: Flow cytometry scatter plots of nanovials without any cytokine capture antibody and nanovials with only IFN-γ or TNF-α cytokine capture antibody are used to identify a positive threshold gates for IFN-γ, TNF-α or IFN-γ and TNF-α secreting cells. Each quadrant represents the following secreting population: Q1) IFN-γ secretors, Q2) IFN-γ and TNF-α polyfunctional secretors, Q3) TNF-α secretors, Q4) non-secretors. FIG. 33B: Flow cytometry scatter plots of T cells loaded onto nanovials labeled with all three antibodies (anti-CD45, anti-IFN-γ, anti-TNF-α) and sorted populations based on calcein signal and presence in one of the four quadrant gates according to each secretion phenotype. Green signal on microscopy images represents calcein signal. The majority of T cells heavily secreted IFN-γ and limited number of cells secreted TNF-α. Polyfunctional T cells (IFN-γ and TNF-α secretors) represented 13.4% of the population and were successfully sorted and recovered. Scale bars represent 50 μm.

FIGS. 34A-34D: Multiplexed secretion profiling combined with cell surface labeling. FIG. 34A: Overview of multiplexed profiling of single-T cells based on cytokine secretion and cell phenotype. T cells were loaded onto nanovials labeled with anti-IFN-γ and TNF-α, or anti-IFN-γ and IL-2 along with anti-CD45. After accumulating secretion over 3 hours following PMA/ionomycin stimulation, captured cytokines were labeled with their respective fluorescent antibodies and cells were stained with fluorescent anti-CD4 and anti-CD8 antibodies, followed by analysis and sorting with a Sony SH800. FIG. 34B: Flow cytometry scatter plots showing CD8+ and CD4+ gates for cells on nanovials, control nanovial scatter plots for secretions without any secretion capture antibodies, and experimental scatter plots showing IFN-γ and TNF-α or IFN-γ and IL-2 secretion on nanovials. The control nanovial scatter plots were used to set the gates for sorting secreting populations of cells on nanovials. FIG. 34C: Single cells were sorted based on CD4+ or CD8+ gates as well as the four quadrant gates shown in B. Scale bars represent 50 μm. FIG. 34D: The population distribution based on secretion is shown as a pie chart for CD4+ and CD8+ cells. There were more polyfunctional CD4+ cells than CD8+ cells.

### DETAILED DESCRIPTION OF ILLUSTRATED EMBODIMENTS

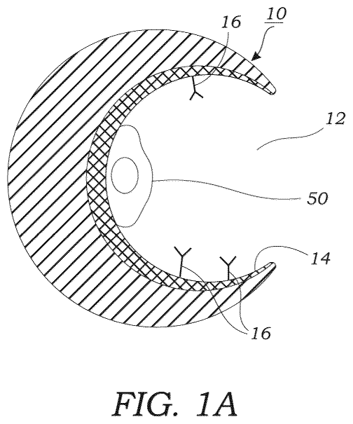

As seen in FIG. 1A, the shaped particles 10 (sometimes also referred to as nanovials) typically are micrometer sized particles (e.g., three-dimensional shaped particles). Generally, the shaped particles 10 have a longest dimensional length of around 100 μm or less. For applications that require the loading of cells 50 into/onto the shaped particles 10, the shaped particles 10 typically have a minimum dimensional length of at least 10 μm. Of course, in other applications, there is no lower limit on the size of the shaped particles 10. In embodiments in which flow cytometers or fluorescence activated cell sorters are used to analyze or sort shaped particles 10, the shaped particles 10 are preferably between ˜30 μm and ˜60 μm in a maximum dimension. The shaped particles 10 may be formed from biocompatible materials or polymers. In one embodiment, the shaped particles 10 are formed from polyethylene glycol (PEG).

The shaped particle 10 includes a void or cavity 12 as seen in FIG. 1A. The void or cavity 12 may have a single opening that opens to the external environment of the shaped particle 10 as illustrated in FIG. 1A. The opening of the cavity 12 is dimensioned to allow cells 50 to enter the void or cavity 12. Alternatively, when cells 50 are not loaded into the shaped particle 10, the void or cavity 12 may be completely enclosed by a surface of the shaped particle 10 (e.g., spherical particle with hollow void or cavity 12 therein). In still yet another embodiment, the shaped particle 10 may also include a particle (e.g., spherical particle) with a number of separate voids or cavities 12 distributed within the shaped particle 10. The shaped particles 10 may have a number of shapes including: crescent shaped, bowl shaped, moon shaped, capsule shaped, concentric sphere shaped. The shaped particles 10 may have a void or cavity 12 sized to fit a single cell 50 or, in some embodiments, a plurality of cells 50. For example, the shaped particles 10 may have a void or cavity 12 with a longest dimension of 10 μm-30 μm. In a preferred embodiment, the void or cavity 12 (or collection of multiple voids or cavities 12) is dimensioned to hold a sub-nanoliter volume of fluid. The fluid may include an aqueous-based fluid. As explained herein, in some preferred embodiments, the shaped particles 10 preferably are designed to carry or hold cell(s) 50 within the void or cavity 12. The cell(s) 50 may be located within the volume of fluid within the void or cavity 12. In some embodiments, the cell(s) may adhere or become adherent to a surface of the shaped particle 10 within the void or cavity 12. Of course, in other embodiments, the shaped particles 10 may not hold cells 50. The shaped particles 10 may hold molecules or other substances with or without cells 50.

In one embodiment, the shaped particles 10 have a localized cell adhesive region 14 that includes a cell binding moiety. The localized cell adhesive region 14 is preferably located along the inner surface of the void or cavity 12, however, the localized cell adhesive region 14 also may extend across the outer surface of the shaped particles 10. The localized cell adhesive region 14 may be functionalized with biotin and/or streptavidin to enable binding directly to biotin or streptavidin labeled cells. Alternatively, or in addition, the localized cell adhesive region 14 comprises cell adhesive or binding moieties specific to other cell surface markers or labels such as antibodies or fragments thereof, extracellular matrix proteins or fragments thereof, aptamers, nucleic acids, oligonucleotides, and the like. In one embodiment, the localized cell adhesive region 14 may include poly-L-lysine (PLL) or PLL and fibronectin.

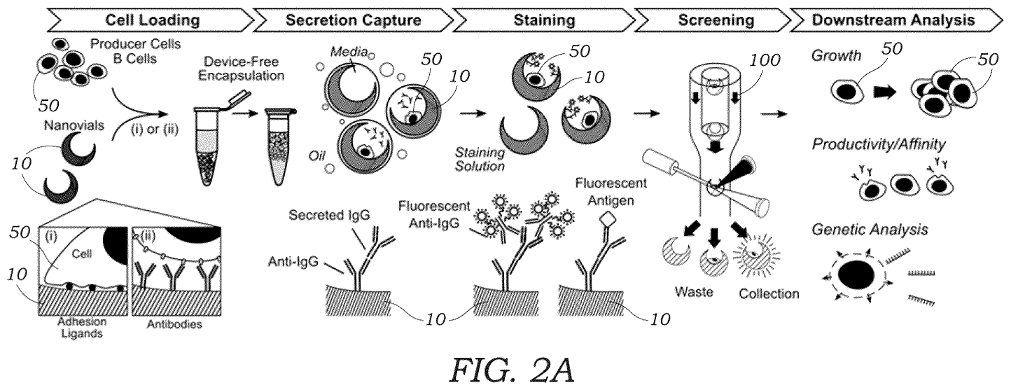

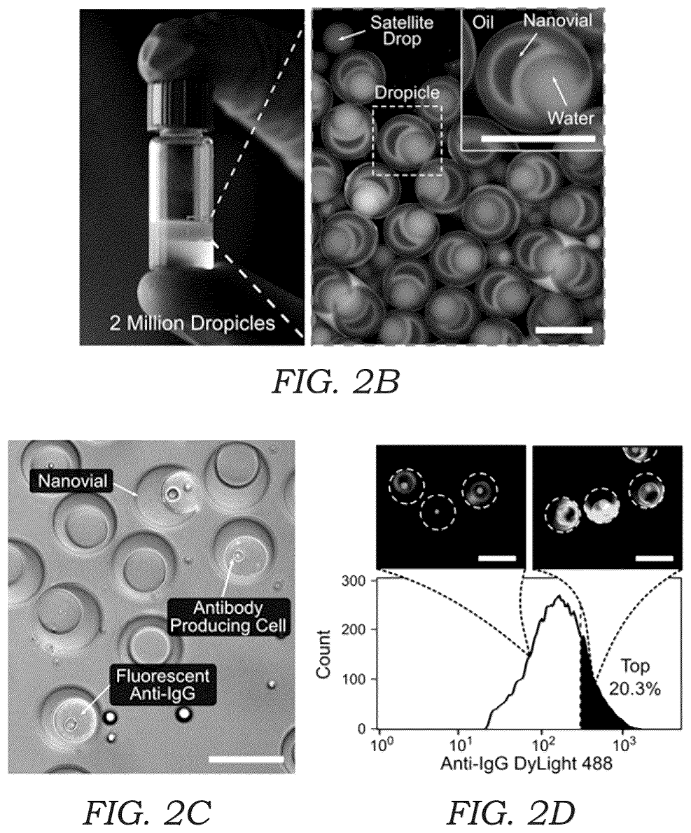

The localized cell adhesive region 14 may be functionalized with one or more affinity capture agents 16. The affinity capture agents 16 may also be incorporated or conjugated throughout the shaped particle 10. The affinity capture agent 16 may include, for example, biotin, streptavidin, a capture antibody or fragment thereof, enzyme, protein, protein fragment, nucleic acid, aptamer, or the like. The affinity capture agent 16 may be secured to the localized cell adhesive region 14 or the shaped particle 10 using a linker molecule such as, for example, biotin and/or streptavidin. By populating the localized cell adhesive region 14 with affinity capture agents 16, the shaped particles 10 can be used to locally enrich secreted biomolecules or other products secreted or released from cells 50. In addition, the localized cell adhesive region 14 populated with the affinity capture agents 16 advantageously reduces unwanted leakage or crosstalk of secreted or released biomolecules interacting with other shaped particles 10. The localized cell adhesive region 14 populated with the affinity capture agent 16 also enables single cell secretion assays to be performed without the need for the formation of shaped particle 10 emulsions (i.e., no need for formation of system in which shaped particles 10 are surrounded by an oil phase). Shaped particles 10 containing cells 50 and secreted or released biomolecules captured on affinity capture agents 16 may be labeled, e.g., with fluorescent reporters (seen, e.g., in FIGS. 2A, 7A, 8A, 10, 11A, 21, 22, 27, 32A, 34A) to characterize the amount, affinity, specificity, or other properties of secreted or released biomolecules.

It should be appreciated that affinity capture agents 16 specific to different biomolecules or products may be contained in a single shaped particle 10 to allow for multiplex analysis. For example, multiple different secreted biomolecules or products may be captured and analyzed in a single shaped particle 10. For example, different fluorescence reporters may be used for the different secreted biomolecules or products that allow for simultaneous downstream analysis using a flow cytometer or FACS instrument 100.

Fluorescent reporters may include fluorescently labeled antibodies, fluorescently-labeled antigens, fluorescently-labeled oligonucleotides, fluorescently-labeled ligands or proteins, enzymes that generate localized fluorescent signal, and the like. Fluorescent reporters (e.g., antigens) may be exposed to shaped particles 10 with captured biomolecules (e.g., antibodies) at different concentrations to probe the affinity of the captured biomolecules to the fluorescent reporter. Generally, the concentration of bound fluorescent reporter (b_{eq}) at equilibrium is linearly proportional to the concentration of captured biomolecules on the shaped particle 10 surface (bₘ), and follows the equation:

$${b}_{e q} = {b}_{m} \frac{{c}_{0} / {K}_{D}}{1 + {c}_{0} / {K}_{D}} ,$$

where the solution concentration of fluorescent reporter is co, and the equilibrium dissociation constant is K_{D}=kₒₙ/k_{off}. Here, kₒₙ and k_{off} are the kinetic association and dissociation constants respectively. Saturating substantially all sites of affinity capture agents 16 on shaped particles 10 with bound secreted or released biomolecules can assist in comparing the equilibrium dissociation constants between secreting cells 50. The concentration of fluorescent reporter can also be adjusted to obtain a high signal for secreted molecules with affinity or equilibrium dissociation constant above a threshold, while a lower signal for with affinity below a threshold. For example, using a concentration of fluorescent reporter approximately equal to or up to 10 times higher than the desired equilibrium dissociation constant threshold leads to significantly reduced bound fluorescent reporter as the equilibrium dissociation constant is reduced, allowing for distinguishing of affinity based on fluorescence signal on the shaped particle 10. In addition, or alternatively, kinetic parameters, such as kₒₙ or k_{off} can be assayed for secreted or released molecules attached to the shaped particles 10 binding to the fluorescent reporter. For both kₒₙ and k_{off} measurements, higher concentrations of fluorescent reporter may be used such as 10 to 100-fold expected or desired K_{D}. Here, the time period for incubation with fluorescent reporter can be used to create a threshold for only high kₒₙ reactions, e.g., by incubations with fluorescent reporter for 30 minutes, 15 minutes, 10 minutes, or even 5 minutes, before washing and fluorescence analysis. To a create a threshold for only low or high k_{off} rates, high concentrations of fluorescent reporter >10 fold expected K_{D} are incubated with shaped particles 10 for >30 minutes or >1 hr. Then shaped particles 10 are washed with a buffer without fluorescent reporter and incubated in the buffer for a period of time, such as 15 minutes, 1 hour, 3 hours, or even 24 hours to allow dissociation of fluorescent reporter from the shaped particle 10, prior to analysis of remaining fluorescence signal. Longer time periods should be used to identify biomolecules with lower k_{off}. The determination of kₒₙ and k_{off} values can be further enabled by blocking antibodies as benchmark or by using different ratios of mutant proteins that can also be labeled. This extends the assayable range of affinities past the general range of nanomolar to high picomolar range either downwards or upwards.

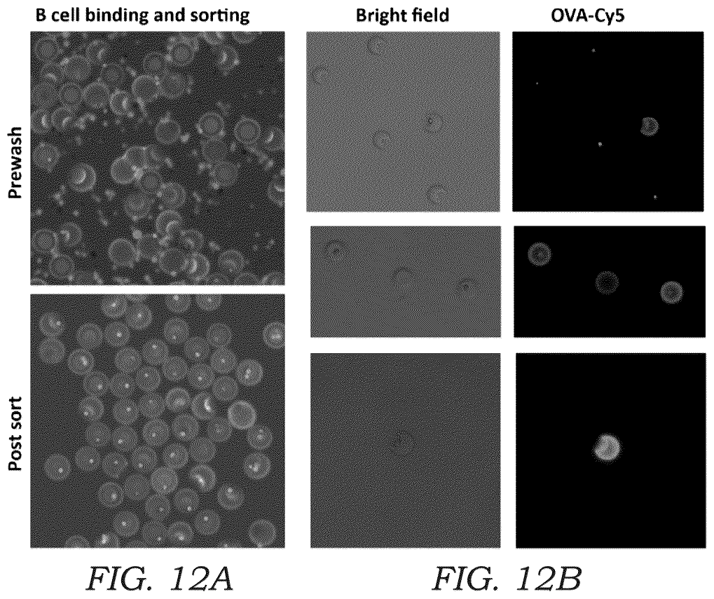

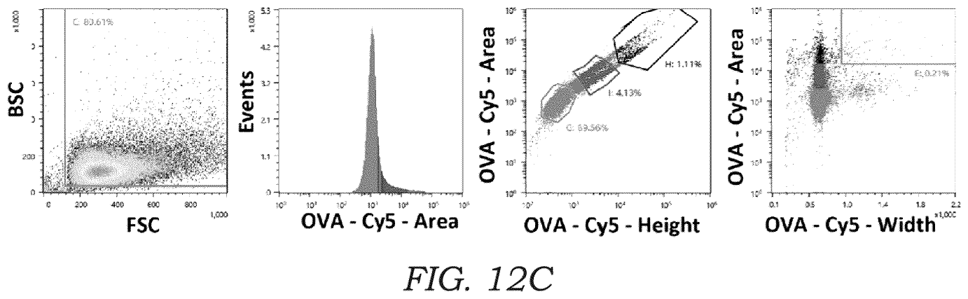

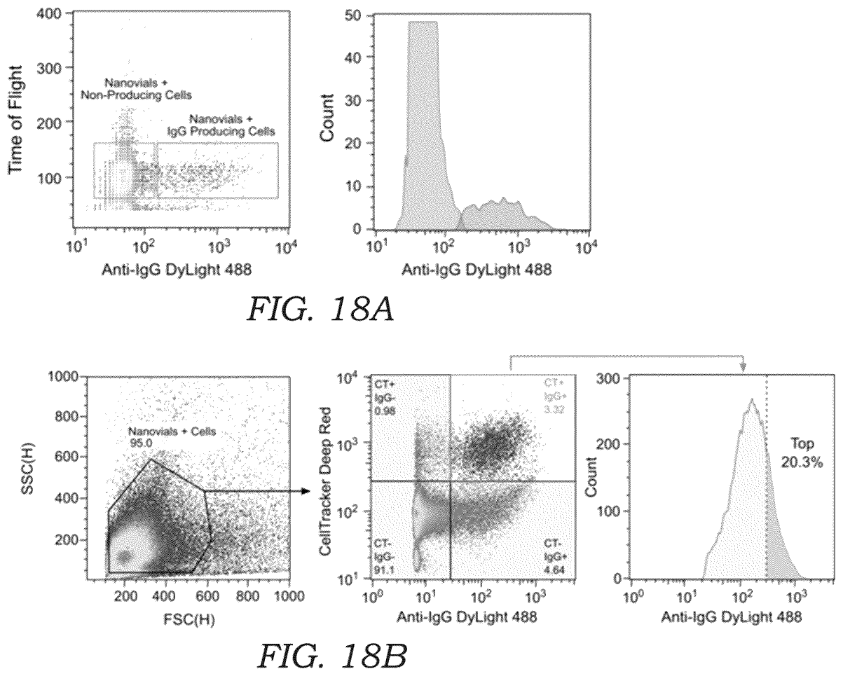

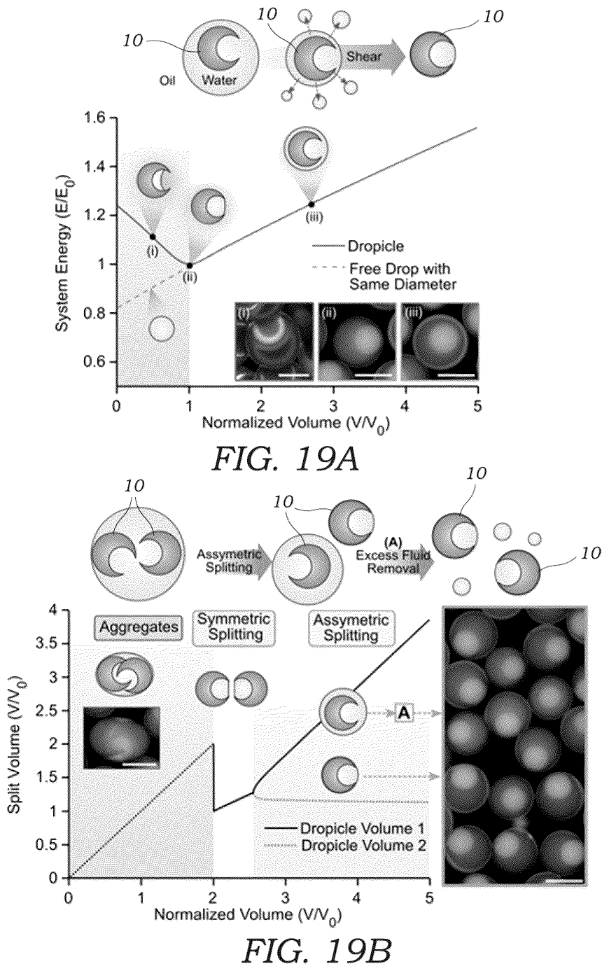

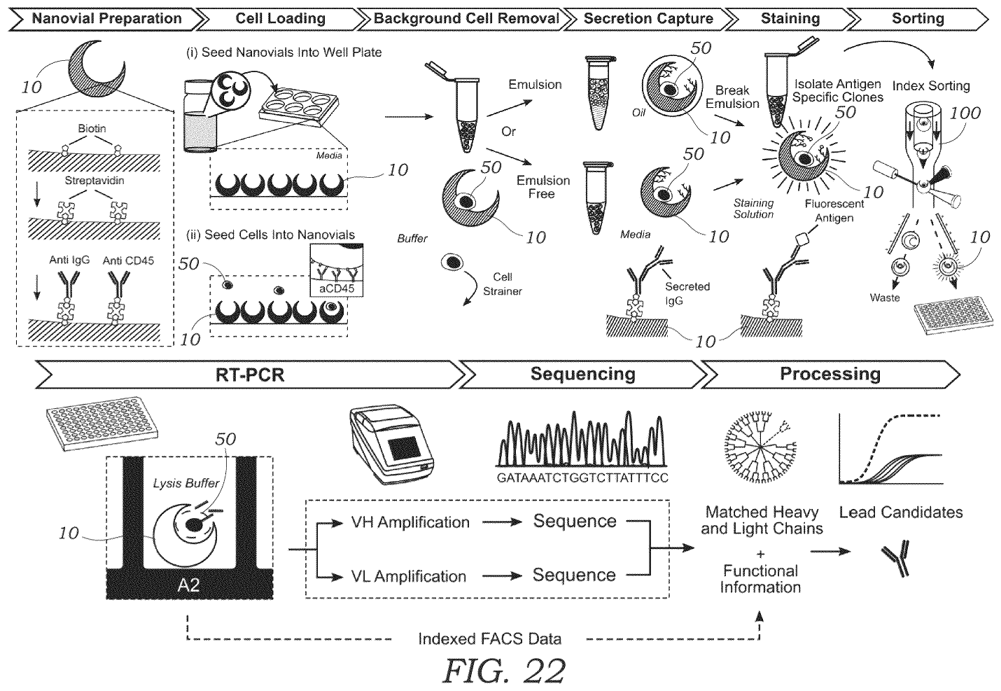

The shaped particles 10 can then be analyzed using a flow cytometer or FACS instrument 100 as seen in FIGS. 2A, 6A, 19, and 20. Data generated from a cytometer or FACS 100 is seen in FIGS. 12C, 18A-18B. The FACS instrument 100 is able to detect an optical signal (e.g., fluorescence) emitted by the shaped particles 10. In addition, or separate from fluorescence detection, scatter measurements may be performed by the FACS instrument 100. This includes forward scatter and side scatter or back scatter measurements. A shaped particle 10 containing a cell 50 and associated secreted or released biomolecules that are labeled with a fluorescent reporter may yield unique fluorescence peak signals when analyzed by the FACS instrument 100. The fluorescence peak signal obtained thereby may be characterized by a fluorescence peak height, fluorescence peak area, and fluorescence peak width. Because of the larger size of the shaped particle 10 compared to a cell 50, the fluorescence peak signals associated with a fluorescently labeled shaped particle 10 may have a larger fluorescence peak width or a larger fluorescence peak area for a given fluorescence peak height when compared to cells alone. These measurements can be used to analyze and/sort the shaped particles 10 containing cells 50 based on the properties of secreted or released biomolecules. A single shaped particle 10 or multiple shaped particles 10 may be sorted into individual wells of a multiwell plate or other container and the fluorescence peak information may be linked or mapped to the location (i.e., the particular well) on the well plate the shaped particle(s) 10 was sorted as seen in FIG. 22. FIG. 22 illustrates a single shaped particle 10 contained in well A2 which has indexed FACS data linked or mapped to that shaped particle 10 and cell 50 contained therein. Shaped particles 10 may also be analyzed using nucleic acid amplification and sequencing reactions (e.g., Sanger sequencing or next-generation sequencing). This analysis may provide information on a nucleic acid sequence of interest from the cell 50 loaded on the shaped particle 10 and associated with specific fluorescence peak measurements. The nucleic acid amplification and sequencing reactions may also be single-cell nucleic acid amplification and sequencing reactions (e.g., single-cell RNA-seq). In some embodiments, the shaped particles 10 may form emulsions in which the shaped particles 10 are located in an aqueous phase and surrounded by a hydrophobic or oil phase. That is say the void or cavity 12 of the shaped particles 10 contains the aqueous phase while the shaped particles 10 are carried in a hydrophobic or oil phase.

FIG. 2A illustrates an exemplary workflow that uses the shaped particle 10 platform for high-throughput single cells secretion screening. Individual cells 50 are loaded (Cell Loading) into prefabricated shaped particles 10 bound using a variety of binding schemes as explained herein (e.g., RGD/PLL or antibodies). The shaped particles 10 and associated cells 50 are agitated by pipetting with biocompatible oil and surfactant (when emulsions are used) to generate monodisperse compartments in the void or cavity 12 dictated by the particle size (dropicles). Crosstalk is thus mitigated between other shaped particles 10. Cells 50 are incubated to accumulate secretions (Secretion Capture) on the associated shaped particles 10 and transferred back to a water phase for fluorescent labeling (Staining). FIG. 2C illustrates a microscopy image of the shaped particles 10 containing an antibody producing cell 50 and fluorescent reporters (Fluorescent Secondaries). The shaped particles 10, cells 50, and associated secretions are then analyzed and sorted using high-throughput commercial flow sorting instruments 100 (Screening). Isolated populations can then be analyzed downstream (Downstream Analysis) for different phenotypic (growth, productivity) and genotypic properties (RNA expression, nucleic acid sequencing) and surface marker patterns which allow for subgrouping of resulting cells 50.

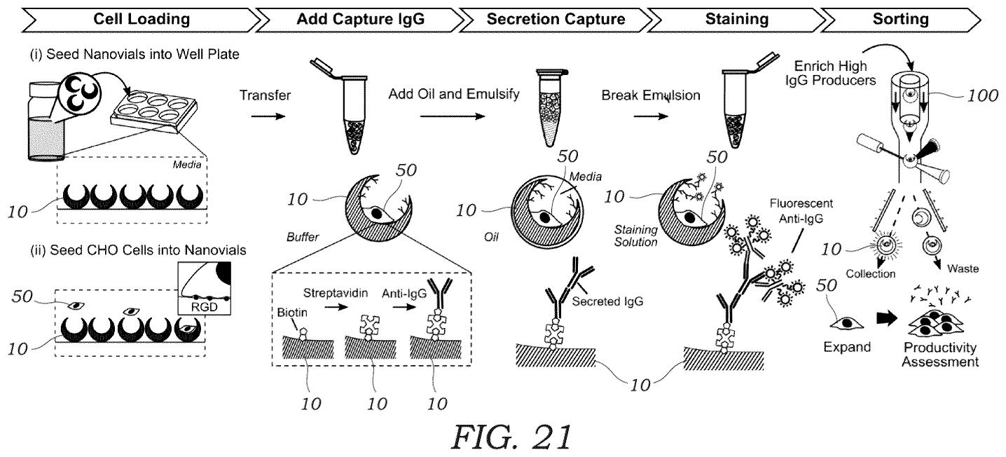

FIG. 21 illustrates an additional exemplary workflow for the enrichment and sorting of high IgG producer cells 50. RGD coated shaped particles 10 are first seeded into a well plate and settle with voids or cavities 12 exposed upright. CHO cells 50 are then seeded into the exposed voids or cavities 12 and incubated to allow attachment via integrin binding (Cell Loading). The shaped particles 12 and attached cells 50 are recovered by pipetting and transferred into a centrifuge or conical tube. The shaped particles 10 are then modified sequentially with streptavidin and biotinylated anti-IgG antibodies (Add Capture IgG). The shaped particles 10 are then emulsified to prevent crosstalk and incubated in a CO₂ incubator and accumulate secreted antibodies from cells 50 (Secretion Capture). The shaped particles 10, cells 50, and associated secretions are transferred back into water phase, labeled with fluorescent reporter molecules (anti-human IgG antibodies with a fluorescent label) (Staining) and analyzed with a flow sorting instrument 100 to measure amount of secreted product. The higher producing cells 50, as measured by fluorescence are sorted (Sorting) out and expanded in a well plate for downstream productivity assessment. The full secretion assay workflow is completed in a single day and cells 50 are expanded for a week after enrichment for downstream assessment via bulk well plate ELISA.

### EXPERIMENTAL

### Precise Fabrication of Suspendable Microcontainers

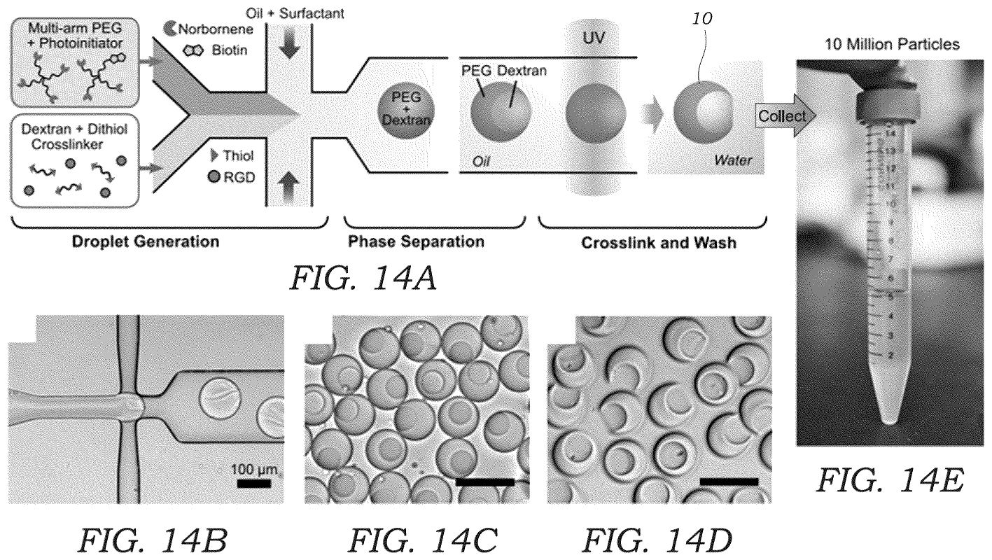

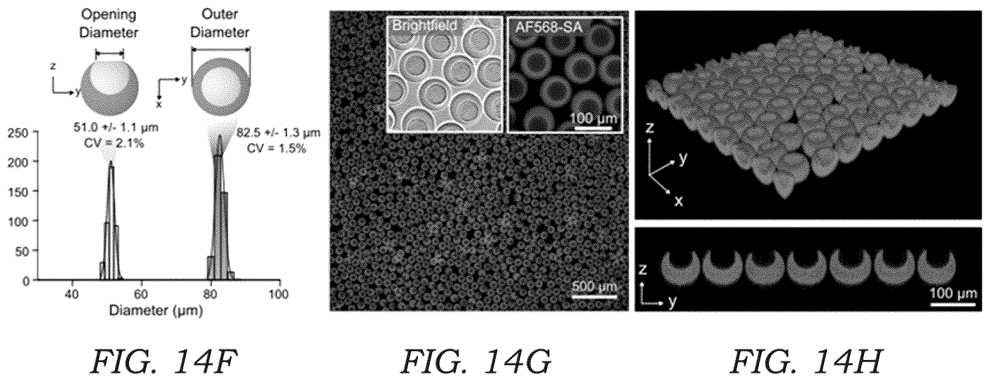

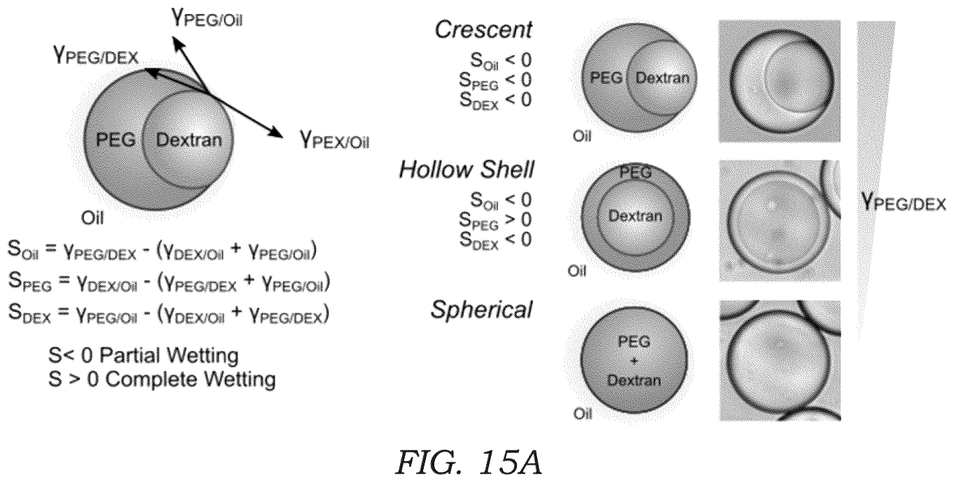

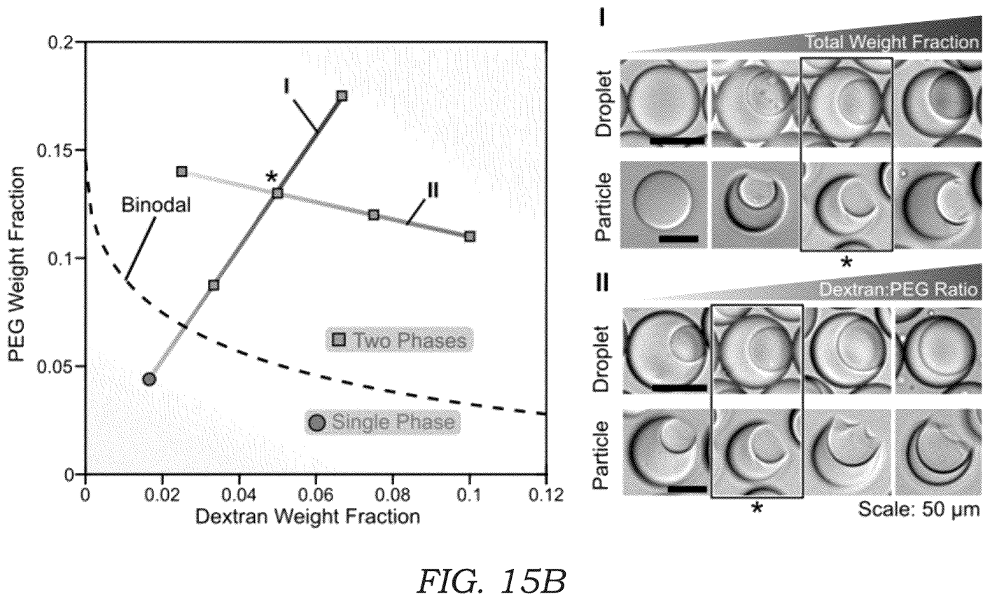

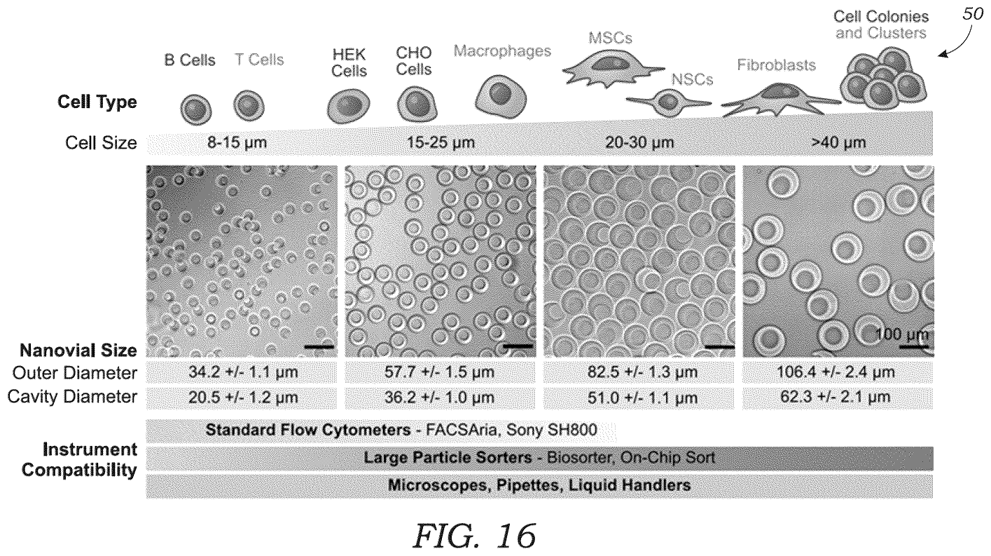

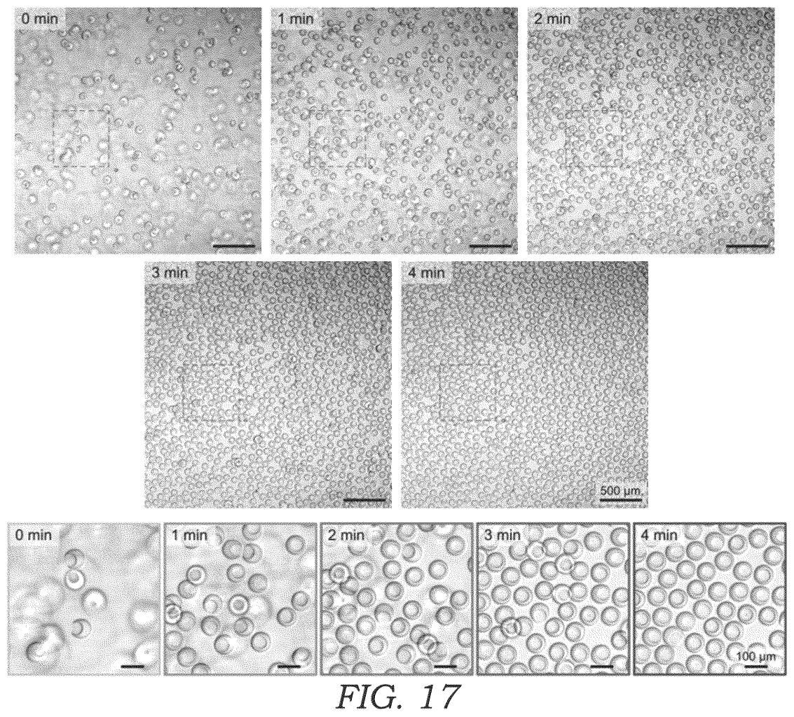

The approach to screen single cells 50 requires shaped particles 10 that are engineered to have voids or cavities 12 to directly load and protect cells 50, functional groups to attach cells 50 and perform chemical reactions, precise shape to prevent cross-talk and loss of secreted signals, and engineered to be compatible with commercial flow cytometers or FACS instruments 100. Utilizing an aqueous two-phase system combined with droplet microfluidics and hydrogel chemistry developed for tissue engineering hydrogel shaped particles 10 (nanovials) were fabricated that meet these requirements at high throughputs (˜1000 s⁻¹) (FIGS. 14A-14H). By tuning the fabrication parameters, one can achieve highly monodisperse polyethylene glycol (PEG)-based shaped particles 10 with accessible internal voids or cavities 12 (outer diameter CV of 1.5%, cavity opening diameter CV of 2.1%, FIGS. 14F-14G). The shaped particles 10 are easily modified with various cell adhesion moieties (e.g., arginine-glycine-aspartate (RGD) peptides, poly-L-lysine, fibronectin or retronectin). Biotin conjugated within the particle matrix allows for facile surface decoration with streptavidin (FIG. 14G) which allows linkage with biotinylated antibodies or antigens for desired secretion assays. This linkage can also be used for antibodies specific to cell surface proteins (e.g., CD45, CD3, CD4, CD8, CD28, and CD19) for cell binding. Monodisperse shaped particles 10 were fabricated to achieve a range of mean diameters (FIG. 16) from 34 to 106 μm which are compatible with a range of cell types and common flow cytometer and FACS instruments 100 (FIG. 16). The morphology of the void or cavity 12 and relative size can be adjusted by tuning the concentration of components in the particle precursor solutions (FIGS. 15A-15B). Importantly, this fabrication step is the only part of the process that requires use of a specialized microfluidic device and is completely separate from the assays themselves. All parts of the single cell assay workflows are performed directly with prefabricated shaped particles 10 and standard lab equipment. This is a critical feature of the platform as it enables the more complex microfluidic/particle fabrication work to be centralized and performed in advance.

### Shaped Particles (Nanovials) as Modular Single-Cell Carriers

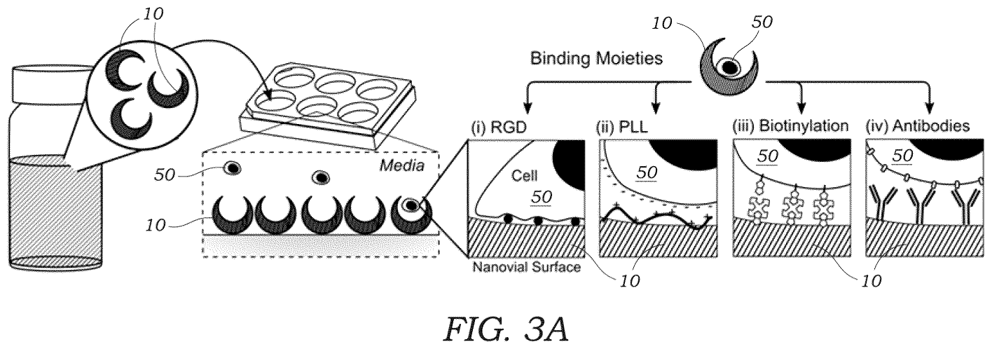

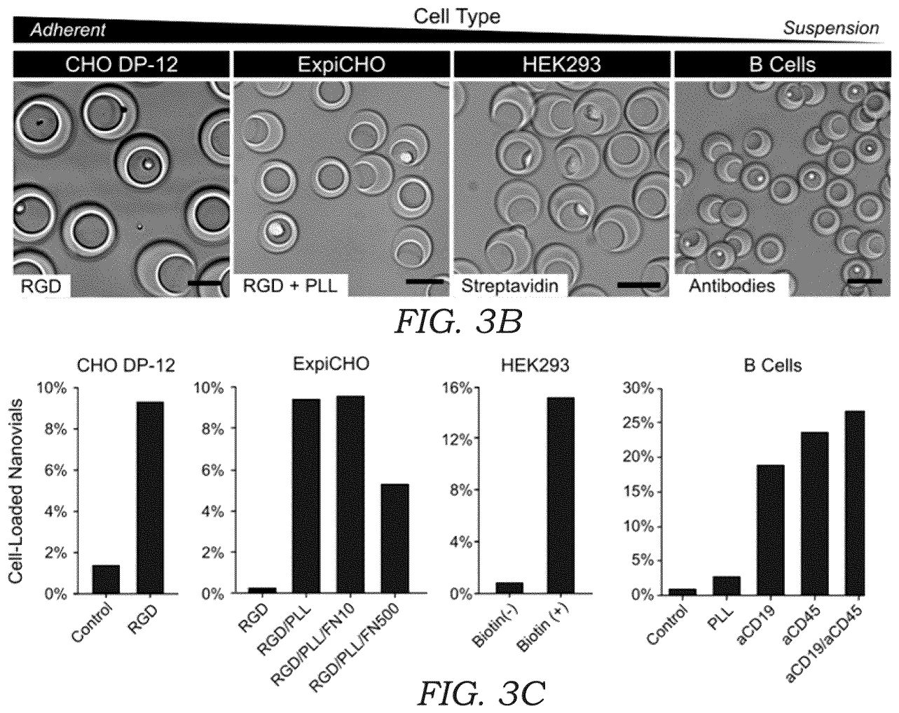

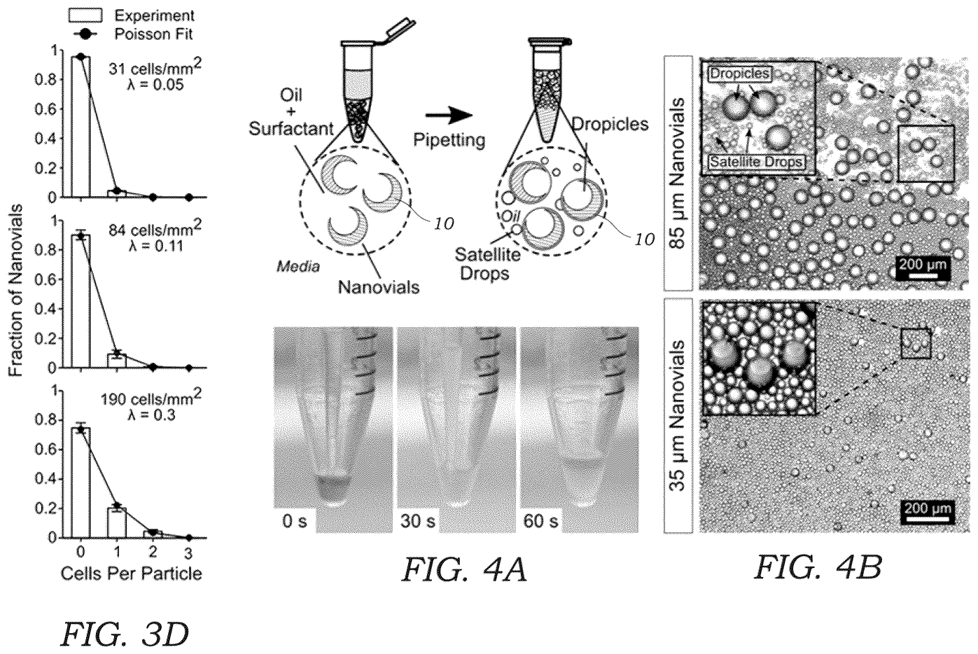

Loading of cells 50 into the voids/cavities 12 of the shaped particles 10 utilizes simple pipetting steps followed by incubation to allow cell binding. Shaped particles 10 can be first loaded into a standard well plate by pipetting and due to their unique morphology settle with their voids or cavities 12 mostly in an upright configuration, forming a monolayer of shaped particles 12 with exposed voids or cavities 12 at the bottom of the well (FIGS. 14G, 14H, 17). Cells 50 are then seeded over the shaped particles 12 and settle with a sizeable fraction coming to rest in the particle voids/cavities 12 (FIG. 3A). It should be noted that this seeding approach led to cell occupancies that closely followed Poisson statistics (FIG. 3D), as is expected for loading of single cells into microfluidically-generated droplets. By controlling the cell seeding density, one can control occupancy such that most shaped particles 10 have either zero (0) or one (1) cell 50 associated with them.

Following seeding, cells 50 are bound to the shaped particles 10 through a variety of cell-surface interactions, depending on the cell type of interest. Adherent producer cell lines used for the production of monoclonal antibodies (mAbs) and other biologics can be adhered through cell surface interactions with RGD, a well-known cell-binding motif present in fibronectin. CHO DP12 and HEK293 cells adhered (FIGS. 3B, 3C) and maintained high levels of single cells 50 within cavities following vigorous wash steps. Suspension-adapted cell lines, like ExpiCHO cells, did not adhere well to RGD alone. However, increased adhesion similar to other cell lines was achieved by modifying the shaped particles 10 to also contain poly-L-lysine (PLL) or PLL and fibronectin (FIG. 3B). For other primary suspension cell populations, such as B cells, it was found that antibodies against cell surface proteins led to optimum adhesion in the voids/cavities 12 of shaped particles 10 even after vigorous washing and sorting. Antibodies against CD45, CD19 and their combination all yielded high levels of B cell, plasma blast, and plasma cell adhesion and maintenance that followed Poisson loading statistics (FIGS. 3B-3D). Additionally, antibodies against CD138 and/or CD38 can be used to enrich plasma cells and plasma blasts.

### Sealing of Shaped Particles Using Biocompatible Oil and Surfactants to Prevent Cross-Talk

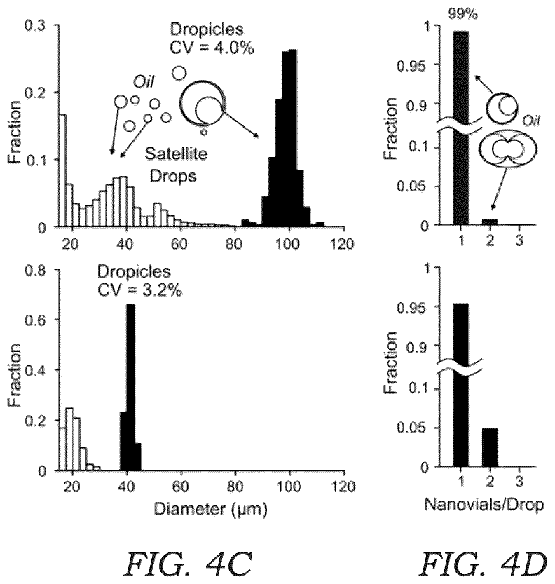

In one embodiment, by exploiting the structure of the shaped particles 10, uniform droplets are formed around the shaped particles 10 via simple pipetting steps with oil and surfactant to seal them and prevent cross-talk between samples (FIGS. 4A-4C). An aqueous solution of shaped particles 10 is first concentrated in a conical or microcentrifuge tube by centrifuging and aspirating the supernatant. A layer of biocompatible oil with surfactant is added and the suspension is then pipetted vigorously for 1 min to create smaller and smaller water-in-oil droplets. Eventually the droplet size is maintained by the outer periphery of the microparticle to form “dropicles” and it was found that, in agreement with minimal energy considerations (FIGS. 19A-19B), uniform volumes of fluid remain stably trapped within the void or cavity 12 by the hydrophilic shaped particle 10. Any excess fluid in the suspension is partitioned into much smaller satellite droplets (FIGS. 4B and 4C). The resulting emulsion shows two unique distributions; (1) small non-uniform satellite droplets and (2) monodisperse droplets each templated by single shaped particles 10 or nanovials (i.e., dropicles) (FIG. 4C). The compartmentalization process was tested over a range of shaped particle 10 sizes (35-85 μm diameters). For larger shaped particles 10 it was found that uniform emulsions (mean diameter of ˜90 microns, CV=4% could be formed with minimal aggregates (<1%, FIG. 4C) from the concentrated shaped particles 10 directly. For smaller shaped particles 10 the addition of biocompatible surfactants in the aqueous phase assisted in breaking the emulsions into smaller partitions. Further dilution of the sample solution aided in reduction of aggregates.

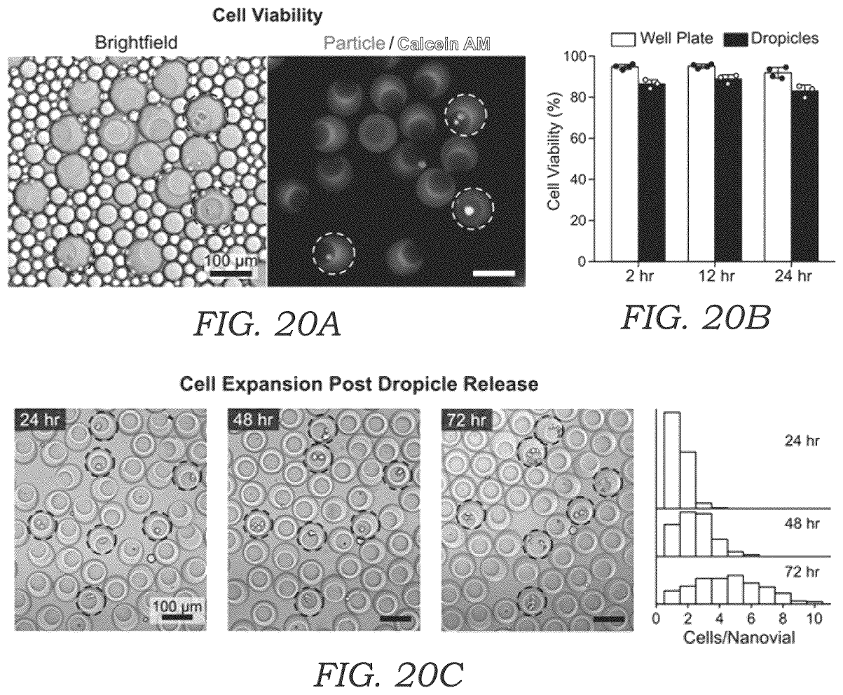

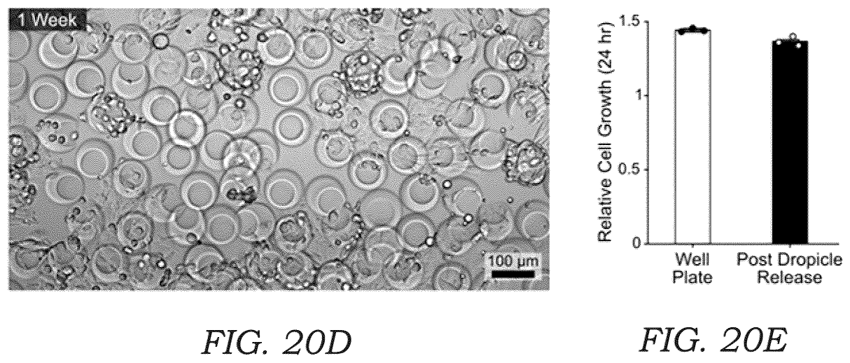

Importantly the shaped particles 10 can be recovered from the oil while still retaining functional cells 50. To recover the shaped particles 10 a biocompatible destabilizing agent is used to coalesce the droplets together. Live/dead analysis of CHO cells 50 following encapsulation and subsequent emulsion breaking shows high viability over a 24-hour period (>80%) indicating that the workflow is biocompatible (FIGS. 20A-20B). Further, similar growth rates from cells 50 expanded after dropicle release in comparison to cells seeded directly into a well plate were observed. (FIGS. 20C-20E). CHODP12 cells 50 adhered via integrin binding and B cells bound via antibodies showed high retention in the voids or cavities 12 of the shaped particles 10 before and after emulsification. After washing and emulsification steps very few cells 50 are found bound to the external region of the shaped particles 10 suggesting that the void or cavity 12 potentially protects cells 50 from shear forces during processing.

### Single Cell Secretion Analysis and Sorting Using Shaped Particles

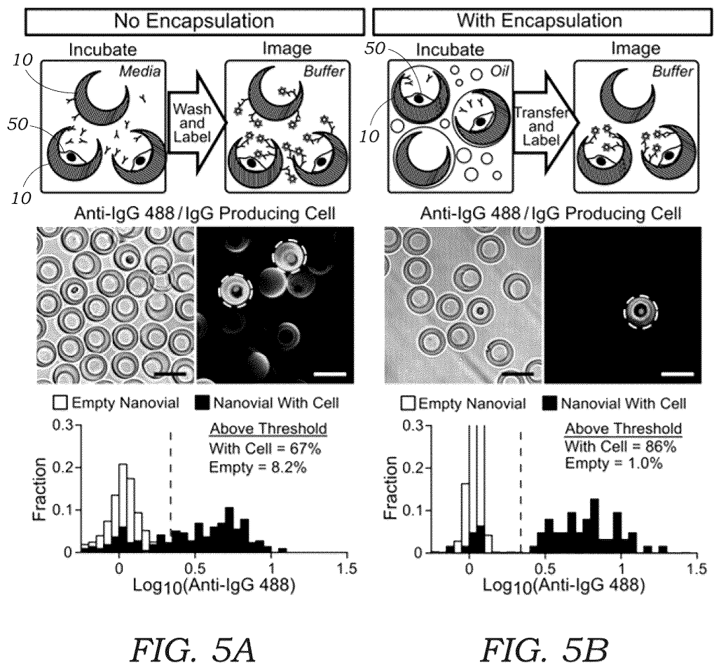

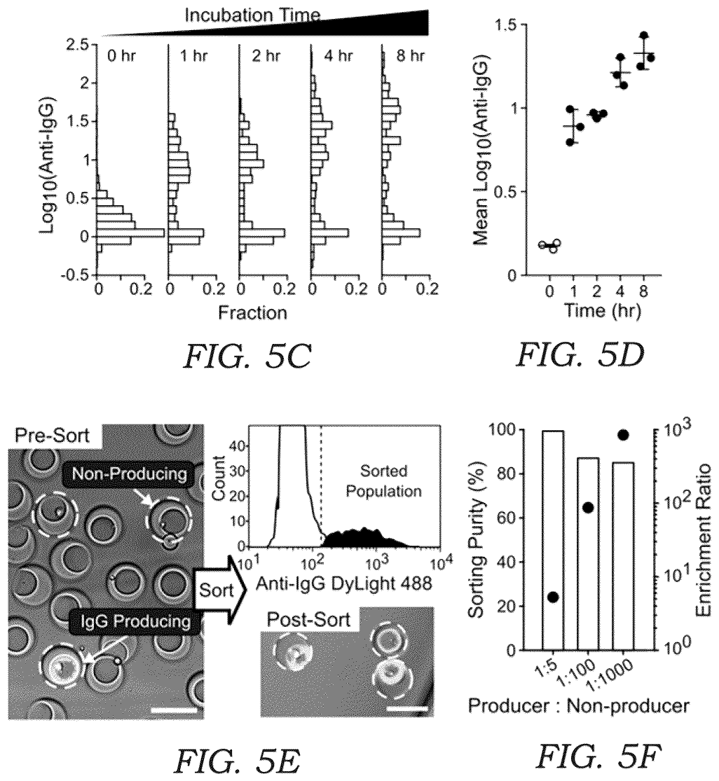

Using the shaped particle 10 platform, a device free workflow has been developed to perform single-cell secretion assays with minimal crosstalk (FIGS. 5A-5B). Given the importance of selecting high antibody titer cell lines for therapeutic production, a Chinese hamster ovary (CHO) cell line was chosen that produces human IgG targeting interleukin 8 (IL-8) and shaped particles 10 modified to capture human IgG as the model system. Cells 50 are first loaded into the voids/cavities 12 of the shaped particles 10 and adhere via integrin binding sites as previously described. After initial cell seeding, the shaped particles 10 and associated cells 50 are collected and washed to remove background secretions. Shaped particles 10 are then coated with anti-human IgG Fc antibodies by binding to biotin groups linked to the particle matrix. Following this step, the shaped particles 10 and associated cells 50 are rapidly compartmentalized in less than 1 minute by pipetting with oil and surfactants. The compartmentalized CHO cells 50 are then incubated, and the secreted antibodies are captured onto the associated particle matrix. After incubation, the emulsions are broken and the shaped particles 10 with attached cells 50 and secretions are collected and washed. The shaped particles 10 with the associated cells 50 and secretions are then labeled with secondary fluorescent antibodies targeting the secreted anti-IL-8 antibodies. Using fluorescence microscopy, retention of cells 50 and associated secretion signal on the particle surface was confirmed after recovering out of oil into an aqueous phase (FIGS. 2C, 5B, 5E, 6B).

Because of the heterogeneity across cell populations, preventing secretion cross-talk between shaped particles 10 is critical to enable quantitative analysis and sorting. When there are high secretion rates, like observed for producer cells 50, rapid encapsulation can aid in to preventing cross-talk of secretions as demonstrated by a side-by-side comparison of the secretion assay with and without the oil encapsulation step (FIGS. 5A-5B). When secretions are captured on shaped particles 10 without the encapsulation step, some shaped particles 10 without associated cells 50 show high secretion signals indicating significant cross-talk (8.2% of empty shaped particles 10 have signal above threshold) (FIG. 5A). Conversely, with the oil encapsulation step there are two visibly distinguishable populations, shaped particles 10 with and without secreting cells 50, and only 1% of shaped particles 10 without cells 50 have signal above the cut off threshold (FIG. 5B). Minimizing the fraction of false positives is particularly critical when secretions can spill over into neighboring shaped particles 10 containing non-secreting cells 50 that would contaminate downstream cultures or sequencing assays.

An increase in mean accumulated fluorescence signal on the 85 μm diameter shaped particles 10 was observed that trends, as expected, with increasing incubation time and corresponding antibody production (FIGS. 5C-5D). Using fluorescence microscopy, one is able to measure significant signal over background (3 standard deviations) after 1 hour of incubation. Interestingly, across all incubation times there are a sizeable population of cells 50 (˜25%) that have no measurable secretion signal (FIG. 5C). Viability analysis of this population revealed that >50% of these cells 50 were still alive indicating that the lack of signal is likely due to a fraction of the cell population no longer expressing the antibody production gene or secreting the produced antibodies.

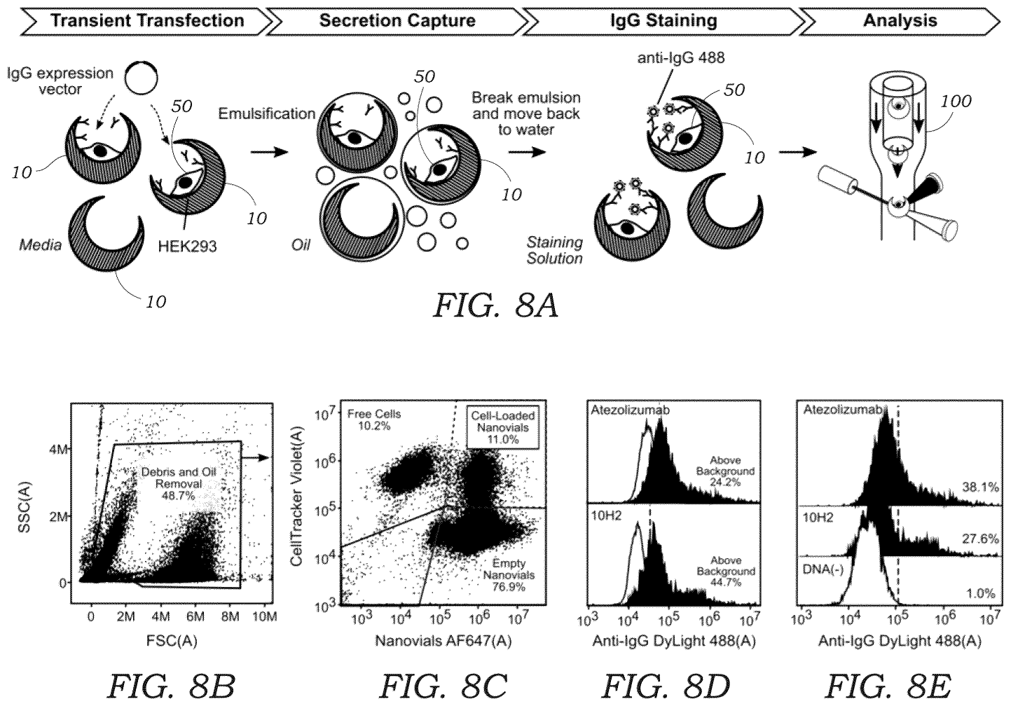

In addition to characterizing IgG production for the stable CHO cell line, the ability to characterize the distribution of secreted product from transiently transfected HEK293 cells was demonstrated (FIG. 8A). HEK293 cells were transfected with plasmids coding for two recombinant IgGs: atezolizumab and 10H2. Cells 50 were first loaded onto streptavidin-coated shaped particles 10 via surface biotinylation of HEK293 cells 50, and plasmid was introduced. Cells 50 and associated shaped particles 10 were then emulsified and cells 50 were allowed to secrete recombinant products for 32 hrs. The emulsion was subsequently broken and shaped particles 10 were recovered for flow cytometry (FIGS. 8B-8C). For atezolizumab and 10H2, 24.2% and 44.7% of cell-containing shaped particles 10, respectively, showed antibody signal above background empty shaped particles 10 (FIG. 8D), demonstrating minimal crosstalk in the system. A long tail of secretion profiles for both atezolizumab and 10H2 was observed, suggesting substantial heterogeneity in the level of gene expression and/or functional secretion. Similarly, 38.1% and 27.6% of cell-containing shaped particles 10 for the atezolizumab and 10H2 transfected conditions, respectively, showed antibody signal above threshold defined by the non-transfected control (FIG. 8E).

To further validate the capability of the system to identify rare subpopulations based on secretions, a FACS enrichment experiment was performed. Secretion-based sorting was performed with a mixed population of the anti-IL-8 producing CHO cells and non-producing CHO cells each labeled with a separate Cell Tracker™ dye (FIG. 5E). After the secretion assay and prior to sorting, fluorescence imaging of the stained 85 μm diameter shaped particles 10 shows increased signal on the particles 10 containing the antibody-secreting cells 50 compared to those containing non-secreting cells 50 (FIG. 5D). This further demonstrates the lack of cross-talk in the system as well as the specificity of the labels only to the secretions of interest. The shaped particles 10 with associated cells 50 and secretions were then sorted based on the labeled secretion intensity (FIG. 5E). Downstream analysis shows effective isolation of the sub-population of interest with high-purity (85-99%) (FIG. 5F) over a range of target cell dilutions. In the most dilute case (1:1000) an enrichment ratio of 850× was achieved indicating the capability to isolate rare target cell events. Cells 50 isolated using this approach can be expanded directly from the particle matrix enabling a streamlined workflow with minimization of trypsinization steps (FIGS. 20C-20D).

### Viable Enrichment of High-Titer Subpopulations Using FACS

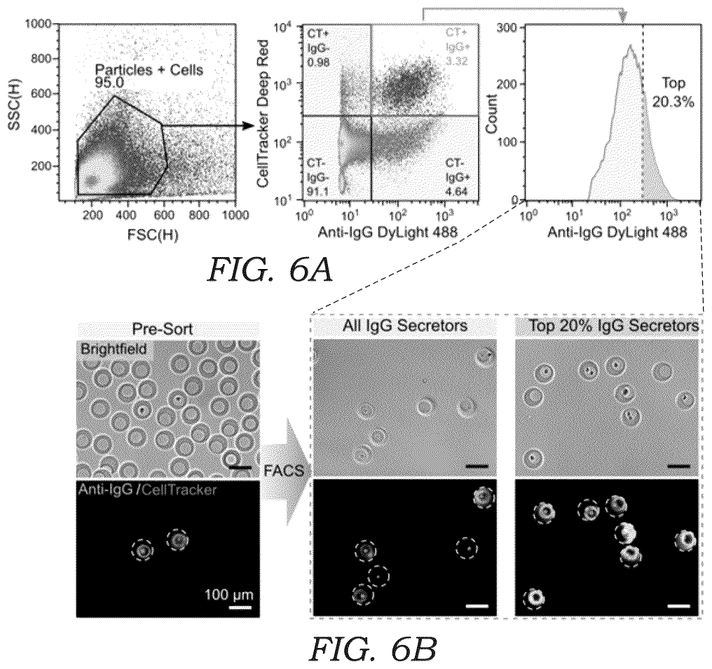

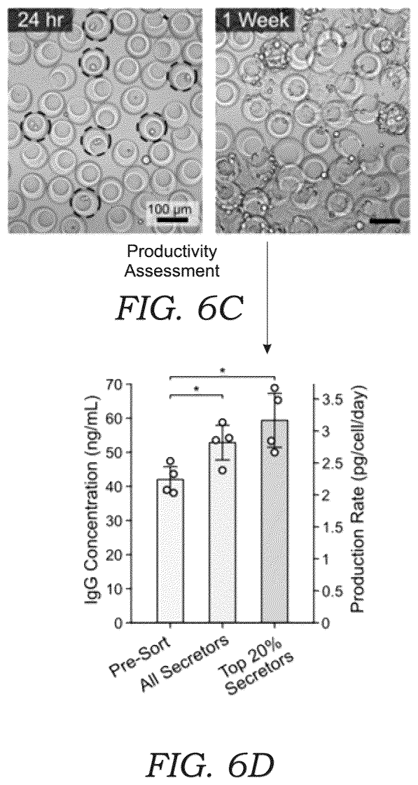

Sorted cells 50 maintain the ability to produce antibodies at levels correlating with the mean intensity of initial secreted signal. In a separate set of experiments with 85 μm shaped particles 10, a secretion assay was performed on human anti-IL8 producing CHO cells 50 and selected out sub-populations based on level of secretion signal using FACS 100 (FIGS. 6A-6D). Cells 50 secreting antibodies were sorted in high-throughput (>200 events/s) by gating off both the fluorescently labeled secretion channel as well as CellTracker™ dye (FIG. 6A, FIG. 16). For each separate passage of cells 50 analyzed (n=4) two sub-populations were sorted: (1) all shaped particles 10 with cells 50 and detectable secretion signal above background and (2) shaped particles 10 with cells 50 and the top 20% of antibody secretion signal. Microscopy images of the shaped particles 10 that were sorted and associated cells 50 show successful isolation of secreting cells 50 with signal proportional to their respective FACS gating (FIG. 6B). The sorted sub-populations were seeded into a 96-well plate and expanded out of the shaped particles 10 over the course of ˜10 days (FIG. 6C). Samples were then plated at the same cell density and bulk antibody production of the different subpopulations was measured by ELISA and compared with non-sorted control samples. It was observed that a 26% increase in total IgG production for the sorted sub-population 1 (all secreting cells) in comparison to the pre-sort control population (FIG. 6D). This increase is most likely due to the removal of cells that are no longer secreting IgG following the sort, which was measured to be ˜25% based on microscopy analysis (FIG. 5C) as well as flow analysis (FIG. 6A). For sorted sub-population 2 (the top 20% of secretors) an average increase of 41% in total IgG production (n=4) was measured with a maximum increase among the samples of 58% (FIG. 6D), indicating the capability of the platform to select out functionally higher producing sub-populations that maintain the phenotype for at least 10 days.

### Isolation of Antigen-Specific IgG Secreting Cells

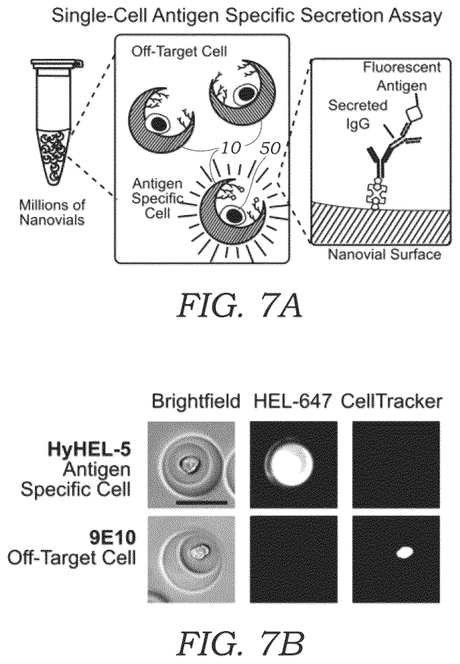

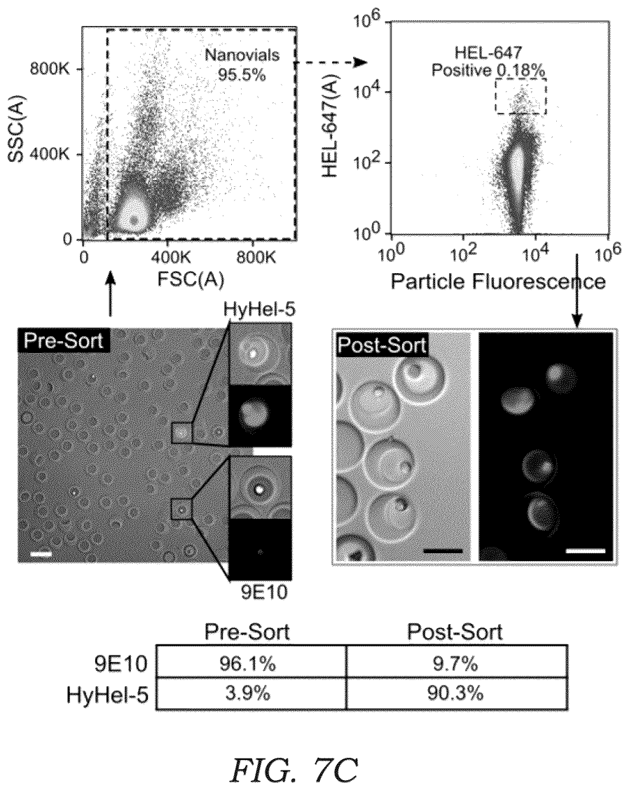

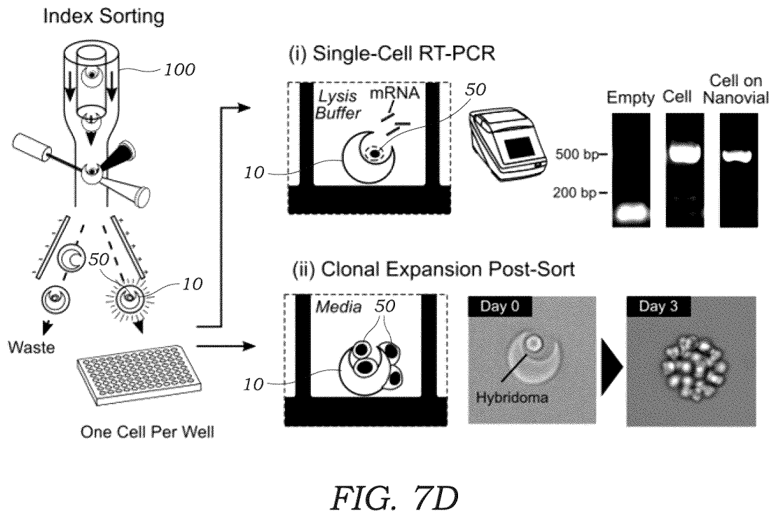

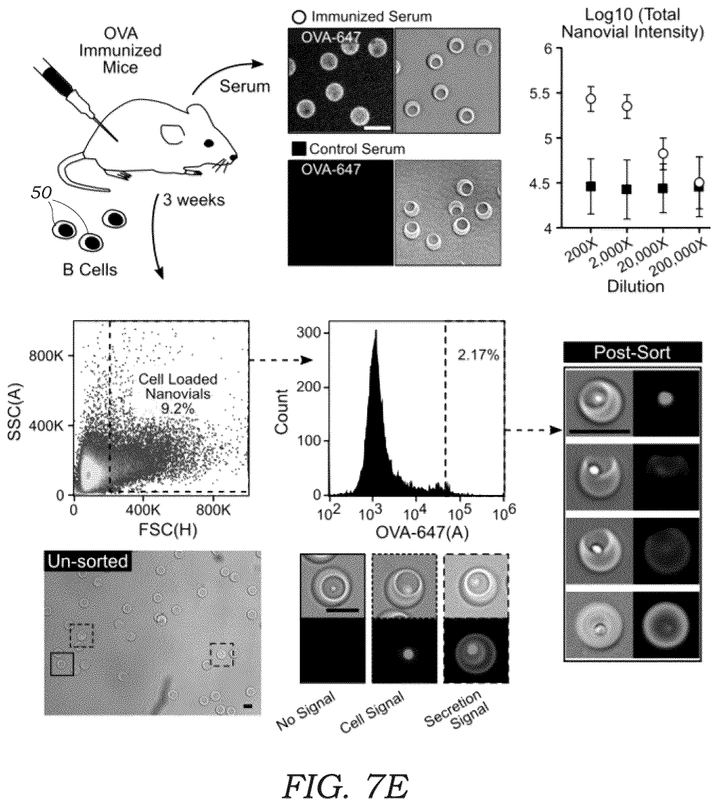

In one application, the feasibility of using the shaped particle 10 platform was tested to screen and isolate antigen-specific antibody producing cells 50 out of a background of similar, non-specific, antibody producers (FIGS. 7A-7E, FIG. 22). Rather than selecting simply for high secretion rates, this type of assay replicates strategies taken for antibody discovery against a target antigen using the nanovial secretion screen. HyHel-5 hybridoma cells secreting anti-hen egg lysozyme (anti-HEL) antibodies (IgG1) were used as the target cell population and were diluted into a background of similar, CellTracker™ Blue (CTB) labeled, 9E10 hybridoma cells 10 producing anti-myc antibodies (IgG1).

Mixed populations of HyHel-5 and 9E10 hybridoma cells 50 were loaded and bound to shaped particles 10 using anti-CD45 cell capture antibodies as described previously, and all antibodies secreted from both cell types were captured non-specifically onto the shaped particle 10 surfaces using biotinylated secretion capture antibodies targeting mouse IgG heavy and light chains (FIG. 7A). Following incubation to capture secreted IgG, particles 10 were labeled with fluorescently conjugated HEL antigen (HEL-647), revealing the presence of only the antigen-specific antibody secretions (FIG. 7A). When assaying mixed hybridoma populations, this assay format yielded strong and specific signal with over 80% of shaped particles 10 containing HyHel-5 hybridoma cells 50 staining strongly for antigen specific secretion after one hour, while no detectable signal was present on shaped particles 10 containing 9E10 cells (FIG. 7B). Over one million shaped particle 10 events were analyzed and a gate encompassing 0.18% of the shaped particle 10 population was sorted based off of their high HEL fluorescence area. Subsequent microscopic analysis confirmed highly enriched populations of strongly labeled HEL-647⁺/CTB⁻ cell-loaded shaped particles 10 in the post-sort population (>90% purity), confirming that the entire analysis and sorting workflow was compatible with detecting antigen-specific antibody produced by target suspension cells (FIG. 7C). In addition to bulk recovery of enriched cell samples producing antigen-specific antibodies, single hybridoma-loaded shaped particles 10 were recovered directly into individual wells of a 96-well plate for downstream sequence recovery and re-growth (FIG. 7D). Individual cells 50 loaded on shaped particles 10 that were deposited into wells were lysed and antibody sequence information was obtained through standard single-cell RT-PCR amplification (FIG. 7D) with similar efficiency (˜50%) compared to freely suspended single cells, indicating that the materials of the shaped particles 10 do not interfere with the RT-PCR chemistry. Single hybridoma clones loaded on shaped particles 10 were able to be sorted into individual wells, culture them and expand them into clonal sub-populations (FIG. 7D).

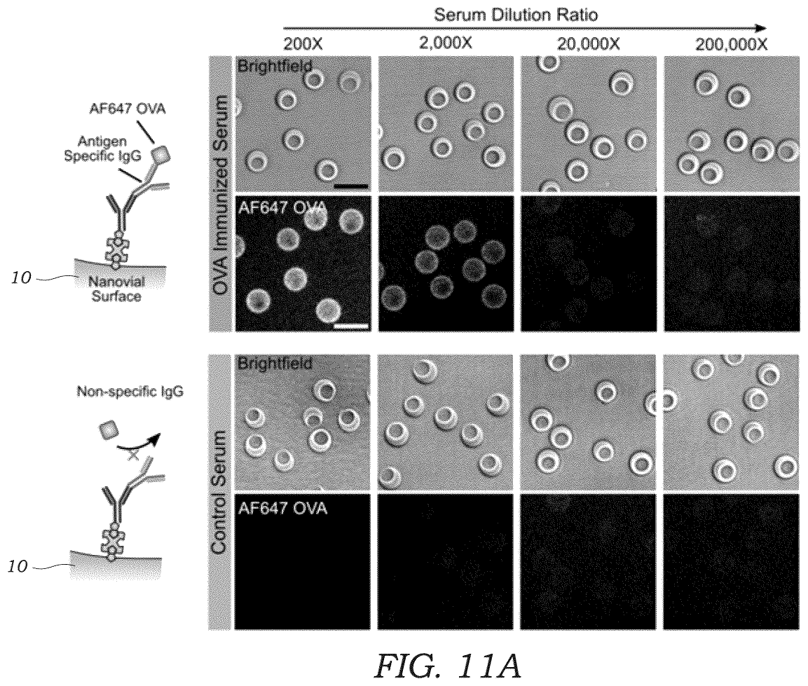

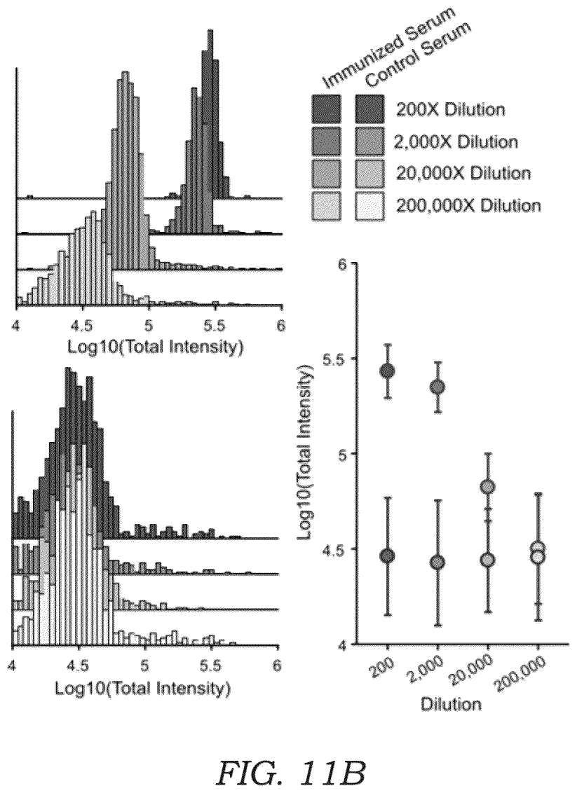

The antigen binding assay was extended to a second antigen and to primary B cells from immunized mice. It was first confirmed that ovalbumin (OVA)-immunized mice produced IgG that was OVA-specific by incubating shaped particles 10 in serum from either immunized or control mice. Anti-IgG coated shaped particles 10 incubated in serum of immunized mice and exposed to fluorescent OVA yielded differentiable fluorescence signal down to a 20,000-times serum dilution, whereas no detectable signal was generated from control serum at any concentration (FIG. 7E, FIGS. 11A-11B). OVA-specific antibody-secreting B cells isolated from the spleens of immunized mice were then loaded and sorted (FIG. 7E). A subset of the analyzed and sorted cell-containing shaped particle 10 was associated with strong OVA-specific signal on the shaped particles 10 themselves, indicating capture of secreted antibodies (FIG. 7E). There was little to no surface stain on the captured cells (FIG. 7E), suggesting the isolation of plasma B cells which canonically lack high levels of B cell receptor (BCR) on the cell surface. Another subset of the B cells acquired strong surface fluorescence after exposure to fluorescent antigen, but did not secrete detectable levels onto the shaped particle 10 surface, suggesting direct binding to BCRs, or non-specific binding to membrane compromised cells. A third population of cells had both cell surface BCR staining and staining on the shaped particle 10 surfaces, indicative of secreted antibodies. The top ˜2% of shaped particles 10 with the highest OVA staining were sorted by FACS and visualized, yielding the same subsets of cell types (secretion signal, cell surface signal, and combined secretion and cell surface signal) that likely reflects the ability to isolate a larger repertoire of B cell populations, including plasma B cells difficult to isolate with standard antigen baiting workflows.

### Selection of Antigen Specific IgG Secreting B and Plasma Cells

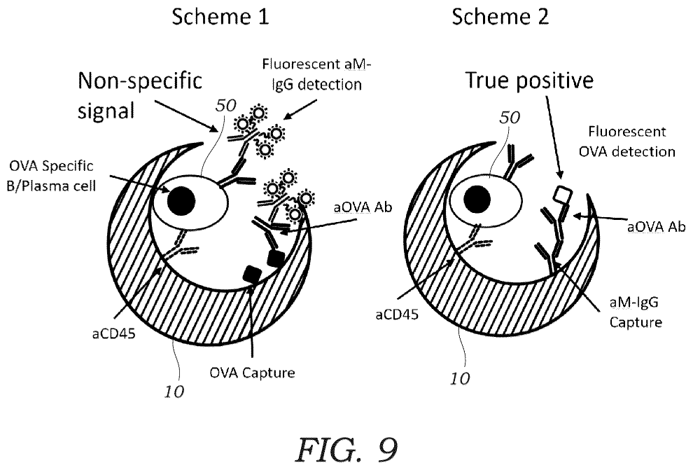

Using smaller 35 μm diameter shaped particles 10, the dropicle platform was applied for the isolation of B lymphocytes, plasmablasts, and plasma cells based on secreted IgG specific to ovalbumin (OVA). Two approaches were developed to assay the antibodies secreted by B cells 50 loaded in the shaped particles 10 (FIG. 9). In the first (FLISA Scheme 1), biotinylated antigen is directly linked to the shaped particle 10 surface and used to capture secreted antibody, and a labeled secondary antibody is bound to the secreted antibody for detection. In the second (FLISA Scheme 210c), a capture antibody is linked to the shaped particle 10 and binds secreted antibody, and labeled antigen is then incubated to yield specific signal when secreted antibodies bind antigen. The amount of signal (i.e., fluorescence) will also depend on the affinity of the secreted antibody to labeled antigen (i.e., fluorescently labeled OVA).

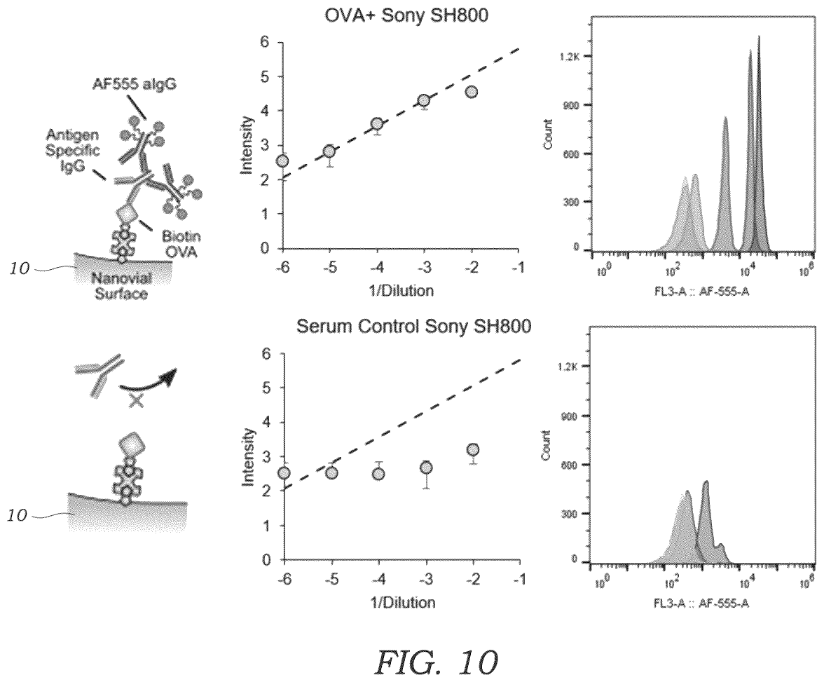

To assess the dynamic range and limit of detection when binding antigen to the shaped particles 10, sera from OVA vaccinated mice was used. Shaped particles 10 coated with antigen were incubated with mice sera diluted from 100 to 2,000,000-fold, and stained with fluorescent anti-Mouse IgG H+L antibodies (FIG. 10). Using fluorescence microscopy, one could detect signal on shaped particles 10 from sera diluted up to 20,000-fold. The sera from non-vaccinated mice had no reactivity with shaped particles 10 at titers as low as 1:10. Assessing the same shaped particles 10 with flow cytometry, the limit of detection was improved by roughly 10-fold, detecting antigen specific antibodies from sera of vaccinated mice at 200,000-fold dilution (FIG. 10).

The second assay format was also tested, capturing serum antibodies with anti-Mouse IgG coated shaped particles 10, and then binding fluorescently-labeled ovalbumin. Using this setup, antigen specific antibodies in sera from OVA vaccinated or immunized mice could be detected at dilutions up to 20,000-fold by fluorescence microscopy (FIGS. 11A-11B). Control serum from mice that were not vaccinated did not show any fluorescence signal above background no matter the dilution amount (FIGS. 11A-11B). The decreased limit of detection here is expected as the non-specific capture method results in most capture sites being occupied by serum antibodies that are not ovalbumin specific.

To test the cell capture and sorting workflow with B cells 50, 35 μm diameter shaped particles 10 were seeded with B cells 50 and sorted at rate of >250 events/sec. The smaller shaped particle 10 sizes were suited to isolate mouse B cells 50 which adhered to anti CD45 antibodies pre-loaded on the surface of the shaped particle 10 through streptavidin-biotin affinity (FIG. 3C) and enabled sorting using a common FACS instrument 100 (Sony SH800) reaching a purity of above 90% cell loaded shaped particles 10 (FIG. 12A). The full workflow was performed to assess antigen specific antibody secretion by plasma cells 50 isolated from the bone marrow of OVA vaccinated mice. 55 μm diameter shaped particles 10 coated with anti CD45 and anti-mouse IgG, for plasma cell binding and secreted antibody capture, respectively, were used. The assay was also performed with CD138+ splenocytes loaded on 40 μm diameter shaped particles 10. After cell binding, dropicles were formed and incubated at 37° C. to capture antibody secretions from encapsulated cells 50. After 2 hours of incubation, the shaped particles 10 were transferred back to water and stained with a fluorescent reporter (Alexa Fluor 647 conjugated ovalbumin). The fluorescence signal on the surface of a fraction of shaped particles 10 containing CD138+ cells (presumably plasma cells, plasmablasts, and some B cells) secreting OVA-specific antibodies was readily visualized using fluorescence microscopy. Furthermore, some cells 50 appeared brightly stained in the images which were not antibody producing cells. The same samples were run on a Sony SH800 FACS instrument 100 and sorted. The shaped particles 10 with cells 50 could be differentiated from empty shaped particles 10 based on a higher back scatter signal (BSC) (FIG. 12C). A subset of events had higher fluorescence (Cy5) area signal and could be gated based on high Cy5 area and/or height (FIG. 12C, gate H). This sub-population could be further gated based on fluorescence (Cy5) width to identify and sort shaped particles 10 with significant staining across their surfaces corresponding to bound antibody secreting cells 50 secreting antibodies with affinity to OVA, compared to cells 50 directly stained with OVA which had lower fluorescence width metrics (i.e., a narrower fluorescence peak). It was also possible to gate and sort sub-populations of shaped particles 10 containing fluorescence signal across the shaped particle 10 by gating for events with higher fluorescence area metrics for a given fluorescence height (FIG. 12B). For example, gating the top 20% or top 10% of fluorescence area for each value of fluorescence height. Gating based on the accumulated fluorescent signal on shaped particles 10 leads to isolation of new populations of cells 50 (e.g., plasma cells) that could not be identified by surface staining of e.g., B cell receptors directly with fluorescent antigen. These cells 50 may have new antibody sequences and/or more mRNA corresponding to the highly secreted antibody, leading to a higher probability of recovering antibody sequence information. The larger number of antibodies bound to the surface of the shaped particles 10 compared to BCRs on a typical cell, may also enable screens based on antigen affinity with a larger dynamic range and limit of detection.

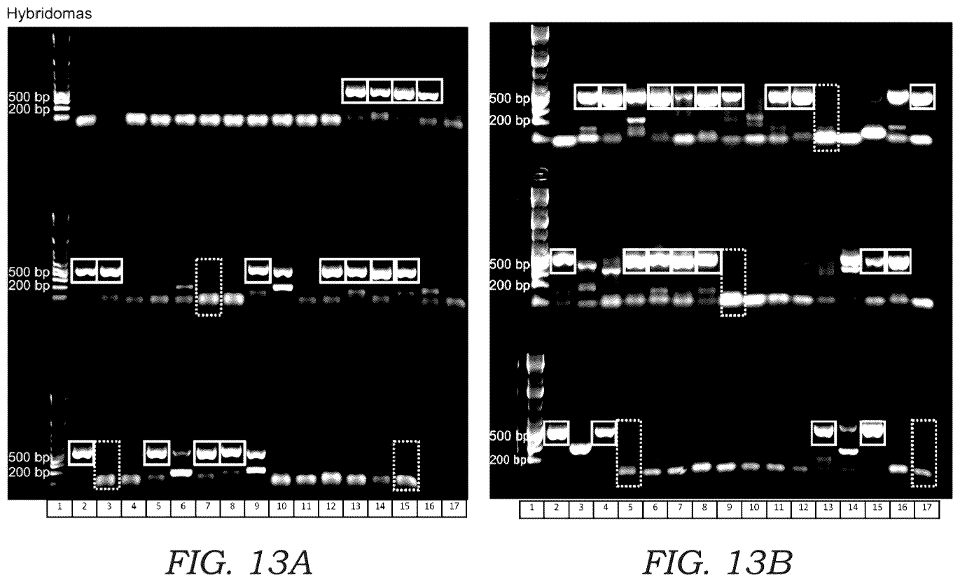

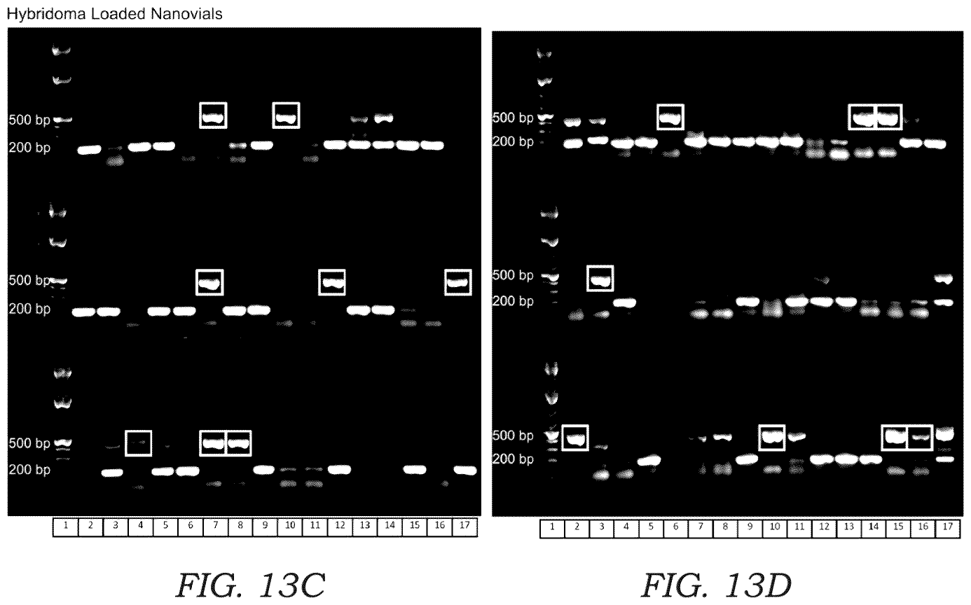

In a separate set of experiments hybridomas derived from B cells 50 adhered on shaped particles 10 were sorted into separate wells of a 96 well plate prepared with reagents for downstream RT-PCR. Cells 50 were lysed and reverse transcription was performed to recover cDNA, followed by nested PCR to amplify the heavy chain and light chain sequences of the sorted hybridoma cells 50 (FIG. 13). The data shows that single hybridoma cells 50 adhered to shaped particles 10 can be sorted in separate wells of a multiwell plate using a FACS instrument 100 and that the shaped particles 10 do not interfere with the reverse transcription or downstream PCR reactions. A large fraction of the wells yield cDNA of the correct length for the expected amplicon. An entire workflow for linking antigen affinity of secreted antibodies from individual B cells/plasma cells/hybridoma cells to antibody sequence information (matched heavy and light chains) is shown in FIG. 22. The workflow includes initial steps of cell loading in shaped particles 10 functionalized with a cell adhesive region 14 and affinity capture agent (anti-IgG) 16 to generate a fraction of shaped particles 10 containing cells 50 therein. Shaped particles 10 are optionally emulsified to reduce secretion leakage and cross-talk between cells 50 in neighboring shaped particles 10. Shaped particles 10 containing cells 50 are incubated to accumulate secreted antibodies with are captured by the anti-IgG. If emulsified, the emulsion is broken and shaped particles 10 are returned to an aqueous phase. Shaped particles 10 are then exposed to a fluorescent reporter for the presence of high affinity IgGs targeting the antigen, in this case fluorescently-labeled antigen. Shaped particles 10 are sorted using a FACS instrument 100 into individual wells of a multiwell plate based on fluorescence signal associated with the shaped particles 10, including fluorescence area, height, and width metrics. Shaped particles 10 with cells 50 that secrete antibodies with affinity to the antigen are selected. These cells 50 are then lysed, mRNA is reverse transcribed, and cDNA is amplified to obtain cDNA for the heavy and light chains of the secreted antibody. The cDNA can then be sequenced, e.g., by Sanger sequencing to obtain an antibody sequence linked to fluorescence signal related to the antibody's function (e.g., affinity).

The shaped particle-based emulsion (i.e., dropicle) platform provides specific advantages in sorting cells 50 based on secretions, but also lays the foundation for the next generation of microfluidic assays on single cells 50 and molecules without the need for new, specialized instruments. Two key features of the shaped particle 10 platform allow for widely accessible analysis and sorting of cells 50 based on secretions: (i) the ability to form uniform compartments (i.e., the void or cavity 12) containing single cells 50 in small sub-nanoliter volumes with minimal crosstalk using simple pipetting and no devices, and (ii) the compatibility of the shaped particles 10 with commercially available flow sorters like FACS and flow cytometer instruments 100 such that viable cells 50 can be sorted based on their associated secretions and remain viable after sorting. Shaped particles 10 allow for seeding of cells 50 into their open voids or cavities 12 and emulsification to form hundreds of thousands to millions of dropicles all in parallel using simple pipetting operations. Parallel encapsulation in <1 min is an important differentiator from microfluidic techniques which often require tens of minutes to several hours to sequentially form droplets while cells remain mixed and secreting in the input sample volume. Rapid emulsification minimizes cross-talk from cells 50 secreting into the mixed input sample volume during the time over which secreting cells 50 are encapsulated which is critical for enabling screening of large cell populations. Dropicle emulsification and analysis steps can also be more easily performed in a sterile environment (e.g., biosafety hood) using standard sterile plasticware (e.g., well plates, pipette tips), as opposed to bulkier equipment. Although microfluidics is used to manufacture the shaped particles 10, the shaped particles 10 can be easily produced in batch (>10 million drop-carrier particles per batch, FIGS. 12A-12H) and provided to other labs who do not have expertise in microfluidics. Shipping of shaped particles 10 is significantly more cost-effective and rapid than replicating microfluidic setups for generation and sorting of droplets. Further, the amount of additional expertise required to use the particle-based system is substantially lower than microfluidic based approaches due to the familiar handling steps.

The shaped particle system is also compatible with commercial flow cytometers which enabled users to screen over 100,000 and in some cases over 500,000 cells in one day of experiments at rates of up to 200 cells/second (or up to 1000 events/second assuming 20% loading). This throughput is comparable to state-of-the-art microfluidic approaches and by further optimizing the loading of cells 50 into the shaped particles 10 as well using higher-throughput FACS instruments 100 this throughput could exceed 10 million cells per day. Besides increasing availability to labs without expertise in microfluidics or specialized commercial instruments, there are some unique advantages of this new approach for encapsulation of mammalian cells 50.

The structure and surface of the shaped particles 10 provide unique capabilities for tuning the number and type of captured cells 50, analysis of adherent cell secretions, and analysis and sorting of clonal colonies. The size and opening diameter of the void or cavity 12 within a shaped particle 10 can be tuned (FIGS. 15A-15B), potentially enabling deterministic loading of single cells 50 based on size exclusion effects. This could enable more rapid screening because cell loading rates may not be limited by Poisson statistics. Besides structural changes to the shaped particles 10, the surface of the shaped particles 10 can also be functionalized with affinity agents that specifically enrich certain populations of cells 50, such as antibodies to CD3 that enrich T cells from a mixed population or antibodies to CD19 or CD138 that enrich B cells or plasma cells from a mixed population. Additionally, antigens or peptide major histocompatibility complexes (pMHC) can be used as moieties to bind cells 50 based on chimeric antigen receptor or T cell receptor affinity respectively. The surface of the shaped particle 10 also enables the attachment and growth of adherent cell populations, such as the CHO cells 50 used herein. Analysis of secretions from adherent cells 50 can be challenging with other microfluidic techniques which require cells 50 to be in suspension to flow into devices and sort afterwards. Adherent cells 50 begin to undergo apoptosis when remaining in suspension and it is expected that secretion rates of biomolecules would change in this condition. The inability to perform these assays has led to a dearth of information on the secretion phenotypes and heterogeneity of adherent cells 50 with important secretion products in health and disease, such as mesenchymal stem cells, glandular epithelial cells, endothelial cells, glial cells, and even adherent bacterial biofilms. Further, using shaped particles 10, when single cells 50 are initially seeded onto the shaped particles 10 they adhere and grow without nutrient limitations. The clonal colonies can then be encapsulated to form dropicles, enabling high-throughput screening based on the combination of growth and per cell secretion (i.e., overall biomass produced per unit time), potentially overcoming previous challenges in cell line selection in which growth and biologic production can be in a trade-off relation. This also enables direct sorting of single shaped particles 10 (and single cells 50 contained therein) into e.g., 96 or 384 well plates with the purpose of expansion of clonal populations with desired properties.

Sorting based on secretions extends beyond selection of high antibody-titer cell lines to many applications of importance in life sciences and biotechnology. Discovering high affinity mAb therapeutics relies on the selection of B cells 50, plasma blasts, and plasma cells producing antibodies with high affinity and specificity amongst a large background, and obtaining a DNA sequence for the matched heavy and light chains. The activity of immune cells is largely connected to their secretion profiles, which direct communication and effector functions and can be better studied by sorting out specific sub-populations for further functional testing in vivo or in vitro. The effectiveness of chimeric antigen receptor (CAR)-T cell batches also appears to depend on a multifunctional secretion of cytokines, such that sorting populations based on secretion profiles may enhance therapeutic activity. Identifying T cell receptors (TCRs) that are specific and lead to functional secretion of cytokines for specific antigen presenting cells are enabled by the described workflow, where the most functional TCR sequences (with matched alpha and beta chains) can be identified that lead to functional cytokine secretion responses. Finally, processes of directed evolution of cell products and cells themselves can benefit from larger numbers of clones being screened, mutagenized, and expanded across multiple cycles using an efficient process relying on standard equipment and expertise.

More broadly, the new approach to encapsulate single entities into uniform compartments with a solid phase can enable a centralized “lab on a particle” platform for a number of single-cell and single-molecule assays. Encapsulation is a key component for single-cell nucleic acid sequencing. Clonal colonies of bacteria, yeast, or algae producing engineered proteins (e.g., fluorescent proteins) can also be maintained in dropicles and sorted based on desirable features (e.g., intensity at particular excitation/emission wavelengths). Moving beyond cells 50, the compartments defined by the voids or cavities 12 can enable digital nucleic acid amplification assays and immunoassays, where the solid phase provides potential for barcoding and capturing of amplified assay signals. Given the ability to rapidly deploy this approach with established lab infrastructure widespread applications of lab on a particle technology across a range of these single cell and single molecule assays are anticipated in the near future. Rapid deployment across the world is of particular importance during emerging pandemics, enabling collective distributed research and development, and removing the bottlenecks of only a few skilled groups being able to contribute to key points in therapeutic and diagnostic pipelines.

### T Cell Cytokine Screening

The ability to measure and compare the relative amount of cytokine secretion from individual T cells massively in parallel has transformative potential for immunological discovery and the advancement of novel cell therapies. T cells are lymphocytes that make up part of the adaptive immune system and are responsible for detecting and clearing pathogenic materials through cytotoxic effector functions while simultaneously releasing secreted cytokine molecules to coordinate the functions of other responding immune cells. Recently, advances in biotechnology have enabled researchers to retarget traditional functions of T cells towards otherwise intractable systemic malignancies, fostering a new era of personalized cell therapies. However, to date, the design rules around the development of novel cell therapies is not well understood. Traditional immune cell phenotyping approaches rely on measures of surface marker expression. However, while these features dictate the primary role of the T cells being assayed, they do not describe the functional capacity of the various cells to carry out that function. By enabling easy measurement of the functional molecules secreted by T cells one can supplement traditional workflows with this critical information and enable both deeper immunological phenotyping to aid basic science and novel highly parallelized assessment of biosynthetic T cell receptor (TCR) or chimeric antigen receptor (CAR) sequence-function relationships to inform the next generation of immunotherapy. Similarly, the approach for engineered receptors can be applied to natural killer (NK) cells.

The shaped particle 10 platform was adapted to profile the secretions from individual T cells 50. Presented herein are details on nuanced variations in shaped particle 10 modification, cell loading, and secretion screening protocols and data showing detection of TNF-α, IL-2, and IFN-γ secreted from individual cells 50 after only 3 hours of incubation. This particle-based approach can also be used for multiplexed detection of several cytokines secreted by individual cells (e.g., T cells or B cells) simultaneously as explained herein. This approach can be used for screening chimeric antigen receptor (CAR) libraries or T cell receptor (TCR) libraries to identify construct designs with maximum cell activation efficacy that trigger functional responses when binding antigen or antigen-presenting cells. That is, the approach can be used along with MHC-peptide modified shaped particles 10 or antigen modified shaped particles 10 (antigens may be directly linked to shaped particles 10 or be present on cells 50 introduced into shaped particles 10 along with T cells) to screen T cell populations containing random variations of CARs or TCRs for function (i.e., cytokine secretion quantity) and sort out high performers for downstream sequence analysis of the high-performing CAR or TCR constructs.

### Particle Modification

T cells can be effectively loaded into shaped particles 10 through the modification of the surfaces of the shaped particles 10 with biotinylated antibodies on a cell adhesive region 14 similarly to methods described previously for B cell and plasma cell loading. Specifically, T cells 50 have successfully been attached onto shaped particles 10 by utilizing anti-CD3, anti-CD28, and anti-CD45 antibodies, but any biotinylated antibody targeting highly expressed T cell surface markers can be successfully adapted using this workflow. The specific choice of antibody should be selected based on user need. Anti-CD3 and anti-CD28 antibodies are T cell specific and are preferred in applications where T cells 50 should be specifically sequestered from a mixture of cells, however common T cell expansion protocols use similar antibodies to induce proliferation and may block a considerable fraction of surface antigens resulting in non-optimal binding. CD45 is a common yet less specific marker shared across lymphocytes, it is expressed highly and does not directly induce T cell activation making it an ideal candidate for studies where users are starting directly with a pure population of T cells for analysis.

To modify shaped particles 10, streptavidin is pre-diluted into the shaped particle 10 wash buffer to a concentration of 300 μg/mL and mixed with an equal volume of particle sample, yielding a final solution of 150 μg/mL of streptavidin. This solution is incubated with the shaped particles 10 for ˜15 minutes to allow streptavidin coating of the surface of the shaped particles 10. Samples are then washed three times and subsequently reacted with biotinylated antibodies for 30 minutes through a similar pre-dilution and mixing strategy. Due to the relatively low secretion rate for cytokines from primary cells compared to other preliminary work using producer cell lines, it was found that it is not a significant issue to react shaped particles 10 with both cells 50 and affinity capture agent (cytokine capture antibodies) 16 simultaneously, thus all anti-surface marker and anti-cytokine antibodies are pre-mixed simultaneously at this step. Through preliminary evaluations of cell binding efficiency and cytokine signal detection it was determined that optimal antibody concentrations for particle modification are 5.5 μg/mL for cell capture (anti-CD45, anti-CD3, anti-CD28 etc.) and 8.25 μg/mL for cytokine capture (anti-IL-2, anti-TNF-α, anti-IFN-γ) antibodies respectively. After the 30-minute incubation of shaped particles 10 with antibodies, samples are washed three times once more and are ready for use.

An alternative strategy that can be employed to load T cells 50 into shaped particles 10 is to first modify shaped particles 10 with streptavidin and to simultaneously coat T cells 50 directly with biotinylated antibodies against surface antigens. The advantage in this method of loading is that cell attachment antibodies will take up only enough streptavidin sites for cells 50 to bind properly, leaving the remainder of the functional handles open for subsequent affinity capture agents 16 used for cytokine binding. It was found that typically higher antibody concentrations are needed to coat cells for adhesion on the surface of the shaped particles 10 using this method.

T Cell Adhesion onto Shaped Particle Surfaces

Loading proceeds similarly to previously reported methods for lymphocyte adhesion into shaped particles 10. First, shaped particles 10 were seeded into a microwell plate in a volume such that a uniform monolayer of shaped particles 10 covers the entire well surface. In standard T cell applications, one typically adds 6 μL of 35 μm shaped particles 10 diluted into at least 1 mL of washing buffer to uniformly cover a 24 well plate. Samples settle through gravity and typically take 30 mins to orient properly at the bottom of the well with their cavities facing upwards. Once settled cells 50 can be added into the same well and allowed to settle into the previously seeded shaped particles 10. It was found that the addition of 122 μL of cell solution at a concentration of 1 million cells/mL yields optimal loading for most assays.

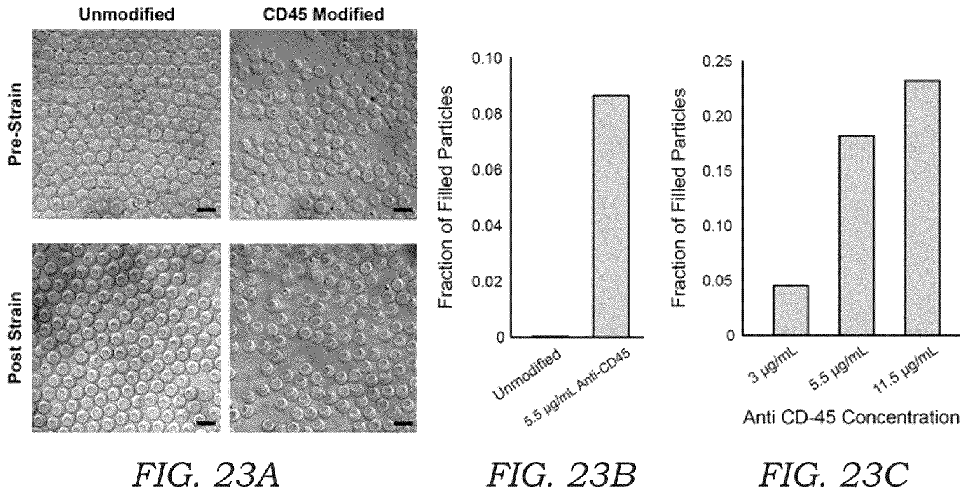

Two slightly modified protocols were tested for loading T cells 50 into shaped particles 10. In the first, cells 50 are added directly on top of previously seeded shaped particles 10 and allowed to settle into the upward facing cavities. Care must be taken in this step to not perturb the settled shaped particles 10 as too strong of a pipetting force may disrupt the shaped particle monolayer leading to large open areas of the well footprint where no shaped particles 10 are present, and no cells 50 can bind. If done correctly, this produces controllable loading fractions and high levels of single cells 50, however this method is more prone to user error especially for smaller sized ˜35 μm diameter shaped particles 10. The effectiveness of loading using this approach was compared by testing the retention of T cells 50 within voids or cavities 12 for both shaped particles 10 modified with 5.5 μg/mL of biotinylated anti-CD45 and all unmodified shaped particles 10. Cells 50 were loaded into each shaped particle condition and allowed to incubate for 1 hour to promote sufficient adhesion. After straining away unbound background cells it was determined that ˜8.6% of anti-CD45 particles retained a T cell within their cavity whereas less than 0.1% of unmodified shaped particles 10 had a cell bound (FIGS. 23A, 23B).

In the second method of loading, the same volume and concentration of cells are added into a well containing shaped particles 10 and the entire cell-particle mixture is successively mixed together with a 1 mL pipette and allowed to re-settle. Rather than loading and incubating for 1 hour as described in the previous approach, cells 50 and shaped particles 10 were mixed in a 24 well plate and allowed to settle for 30 minutes, after which time the entire volume was resuspended a second time and allowed to incubate for a subsequent 30 minutes. In this manner, cells 50 that initially were unable to fall into a void or cavity 12 can be redistributed and given a second chance to enter a shaped particle 10. This method will yield more cells 50 within particle voids or cavities 12 than the previous approach but will also give a higher fraction of cell multiplets especially if particle voids or cavities 12 are much larger than cell diameters. This method was tested with anti-CD45 antibodies at low (3 μg/mL), medium (5.5 μg/mL), and high (11 μg/mL) concentrations. Again, after straining away of background cells a clear differentiation between the three conditions was seen, with roughly 4.5%, 18%, and 23% of shaped particles 10 containing T cells 50 for the low, medium, and high conditions (FIG. 23C). Notably for the same concentration of antibody as used in the previous method there was an over two-fold increase in cell loading with the resuspension method. For the purposes of secretion assays 5.5 μg/mL was used as the optimal concentration for cell retention as it was the lowest concentration of antibodies that yielded robust cell adhesion, leaving as many sites as possible open for anti-cytokine antibody functionalization.

### Cytokine Detection

To characterize the limit of detection and range of cytokine concentrations that can be measured on the shaped particles 10, the variation in signal acquired for shaped particles 10 co-incubated with recombinant cytokine proteins was initially assessed. Three separate cytokines (IL-2, TNF-α, IFN-γ) were screened in this approach. Shaped particles 10 were initially split into three batches and each group was modified with capture antibodies against the associated cytokine of interest as described above. Once modified each group was again sub-divided evenly into six samples and each sample was reacted with a pre-diluted concentration of cytokine ranging from 0-1 μg/mL. After samples were incubated for at least an hour, they were recovered, washed three times and stained with fluorescently-labeled secondary antibodies against the target cytokines.

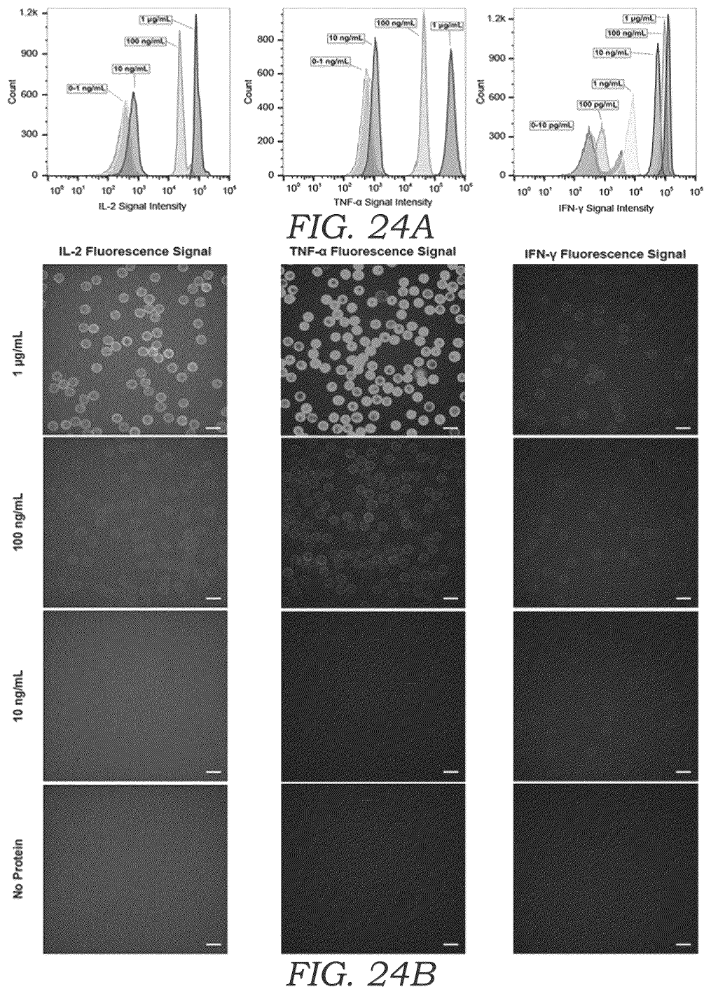

High concentration (1 μg/mL) samples of each cytokine yielded extremely bright signals on shaped particles 10 when analyzed through both flow cytometry (FIG. 24A) and fluorescence microscopy (FIG. 24B), while negative control samples yielded no detectable signal using either analysis method. This indicated that specific detection of each cytokine target was possible on the surface of the shaped particles 10. The observed limit of detection was 10 ng/mL for both IL-2 and TNF-α, and 1 ng/mL for IFN-γ using flow cytometry, and an order of magnitude higher (100 ng/mL for IL-2 and TNF-α and 10 ng/mL for IFN-γ) using fluorescence microscopy. These concentrations were in line with literature reports of similar technologies used to assess protein production from individual cells, indicating compatibility of the system with measuring single-cell cytokine production.

Measuring Cytokines Secreted from Individual T Cells

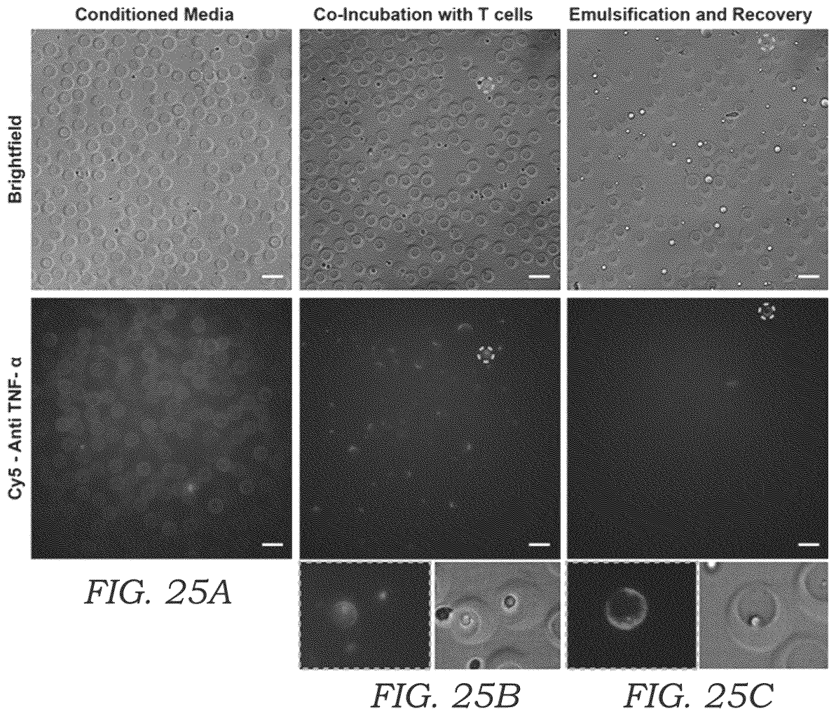

After verifying that the shaped particles-based system was capable of measuring various concentrations of recombinant cytokine products focus was shifted to measuring cytokine production directly from activated T cells 50. For the purposes of these studies, optimization of the secretion assay for a single cytokine, TNF-α was a main focus. Before running the full secretion assay workflow, it was first verified that one could detect TNF-α production from activated T cells in bulk by performing on-nanovial ELISAs both by using T cell conditioned media and by co-incubating nanovials and activated T cells together in a single well. T cells were activated at a concentration of 1 million cells/mL in RPMI media supplemented with a mixture of 10 μg/mL phorbol 12-myristate 13-acetate (PMA) and 25 mM ionomycin. Both samples were assayed three hours after activation. Shaped particles 10 were used to assay conditioned media samples yielded a dim but uniform fluorescence across all shaped particles 10 (FIG. 25A). Interestingly, shaped particle samples co-incubated directly with activated T cells 50 over the same three-hour period also displayed positive TNF-α signal that was localized to specific shaped particles 10 regions (FIG. 25B). T cells 50 locally released cytokines that bound to capture antibodies on neighboring shaped particles 10.

Once it was verified that the T cells 50 were indeed secreting and that the particle-based system could detect cytokines at biologically relevant concentrations following secretion in situ a full T cell secretion assay workflow was carried out with the shaped particle-based system. T cells 50 were activated for three hours using PMA and ionomycin as described above and seeded into anti-TNF-α and anti-CD45 modified shaped particles 10 as described in the previous section. After adhesion non-bound cells were strained out using a 20 μm cell strainer and the mixture of shaped particles 10 and shaped particles 10 containing T cells 50 were recovered. The shaped particles 10 were then centrifuged down to concentrate samples and diluted into a 15× volume of RPMI media. Subsequently, Novec™-7500 oil containing 2% Pico-Surf™ fluorinated surfactant was added to the mixture in a 2× excess in volume over the aqueous phase. The entire sample was pipetted vigorously ˜200 times to generate monodisperse emulsions templated by the hydrogel shaped particles 10. Samples were capped with a small volume of light mineral oil to prevent evaporation and subsequently stored in a 37° C. incubator to allow accumulation of secreted cellular TNF-α.

At the conclusion of 3 hours, 5 mL of fresh RPMI media was added to each sample tube and the light mineral oil was removed from the top layer of the sample volume. Next any remaining un-emulsified Novec™ oil was removed from the bottom phase of the sample and replaced with fresh Novec™ without Pico-Surf™ added. Emulsions were then destabilized through the addition of 20% 1H,1H,2H,2H-Perfluoro-1-octanol (PFO) to each sample (volume the same as oil volume used in initial emulsification step). Finally, the top media phase of each sample was recovered, washed twice, and stained with secondary antibodies according to manufacturer instructions (typically ˜5 μL of 50 μg/mL antibodies per sample). Subsequent fluorescent imaging revealed clear signal localized to the voids or cavities 12 of shaped particles 10 containing cells 50, further demonstrating the capability of this assay to detect and measure cytokine secretions from individual cells 50 (FIG. 25C). Clear fluorescence signal was noticed around the periphery of the cells 50 assayed, highlighting that out workflow can detect cell membrane bound TNF-α precursors as well. By combining this with flow cytometry signal metrics such as fluorescence peak height, peak area, and peak width, the shaped-particle platform will also be capable of differentiating and multiplexing surface expressed and secreted antigen signals. A larger fluorescence peak width, or larger fluorescence peak area for the same fluorescence peak height, can be seen in shaped particles 10 containing labeled secreted cytokine, because of the larger width of the shaped particles 10 compared to cells.

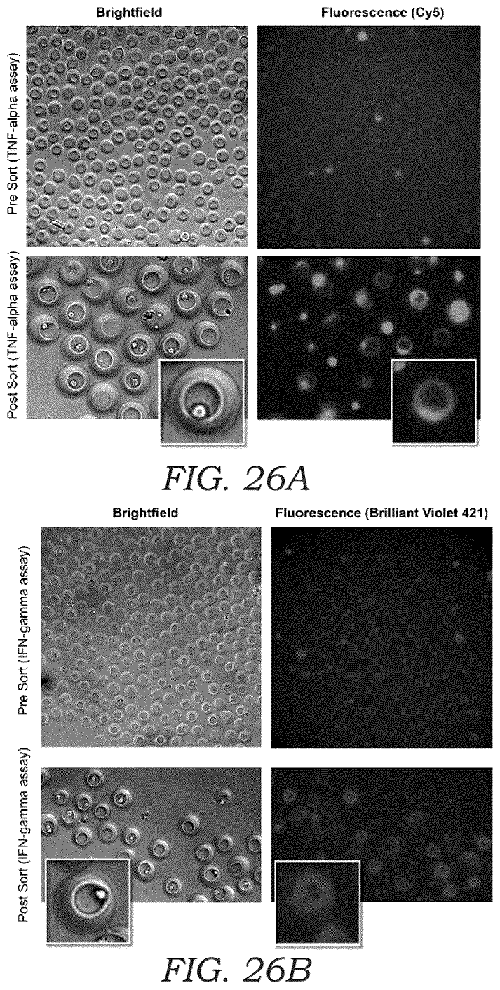

Lastly, for T cell applications, a shortened secretion assay workflow can also be utilized and may be preferred due to ease of use. It was found that by simply attaching cells 50 to shaped particles 10 and straining away background cells, TNF-α signal can be acquired on particles with minimal cross-talk within the same three-hour incubation time frame, without emulsification steps (FIG. 26A). In this shortened version of the assay a preliminary activation step is non-needed, bound cells 50 are activated directly after straining away unbound background cells reducing the two 3-hour incubation steps from the full protocol to one activation and signal acquisition step. Shaped particles 10 with TNF-α secreting T cells were then analyzed by flow cytometry (Sony SH800) and sorted based on high fluorescence peak height, and high fluorescence peak area. Sorted shaped particles 10 show localized fluorescence labeling the voids or cavity 12, and in some cases labeling the cell 50. A single T cell IFN-γ secretion assay was also run using a similar protocol without emulsification yielding strong secretion signal observable by microscopy and flow cytometry (FIG. 26B). Shaped particles 10 with high signal could be FACS sorted based on high fluorescence peak height and high fluorescence peak area, or may be sorted by fluorescence peak width. Sorted shaped particles 10 contained cells 50 and fluorescent signal in the void or cavity 12 of the shaped particles 10 corresponding to secreting cells 50. Cells 50 generally did not become labeled as interferon gamma does not have a membrane bound form as does tumor necrosis factor alpha.

### Multiplexed Analysis and Sorting of Viable Polyfunctional T Cells

Immune cell function is intrinsically linked to secreted factors which enable cells to communicate with neighboring or distant cells to coordinate a response. The ability to secrete cytokines also can help define the population of cells with therapeutic potential in emerging cell therapies, such as chimeric antigen receptor (CAR)-T cell therapies. Polyfunctional cells that can secrete more than one cytokine have been found to play an outsized role in therapeutic efficacy. Here, the shaped particles 10 or nanovials were used to analyze and sort T cells based on a combination of secreted factors, interferon gamma (IFN-γ), tumor necrosis factor alpha (TNF-α) and interleukin 2 (IL-2) and surface markers (CD8+ and CD4+). Cells 50 are selectively loaded into the antibody-functionalized cavity 12 of hydrogel-based shaped particles 10, where secreted cytokines are captured and fluorescently stained. By leveraging a standard fluorescence activated cell sorter instrument 100 and using fluorescence peak shape information one can distinguish between fluorescence signals on the particle cavities 12 and on cells 50 and are able to process greater than 1 million shaped particles 10 in one hour of sorting. A larger percentage of CD4+ cells 50 secreted more than one cytokine than CD8+ cells 50. CD8+ cells 50 that secreted more than one cytokine, were biased towards IFN-γ and TNF-α with fewer CD8+ cells 50 secreting IL-2. The majority of cells 50 with a polyfunctional phenotype that were sorted remained viable and regrew following sorting. This nanovial cytokine secretion assay can be applied to sort antigen-specific T cells 50 or CAR-T cells 50 based on functional engagement with cognate antigen or peptide-major histocompatibility complex (MHC), enabling discovery of functional CARs or T cell receptors and deeper investigation into the molecular underpinnings of single T cell function.

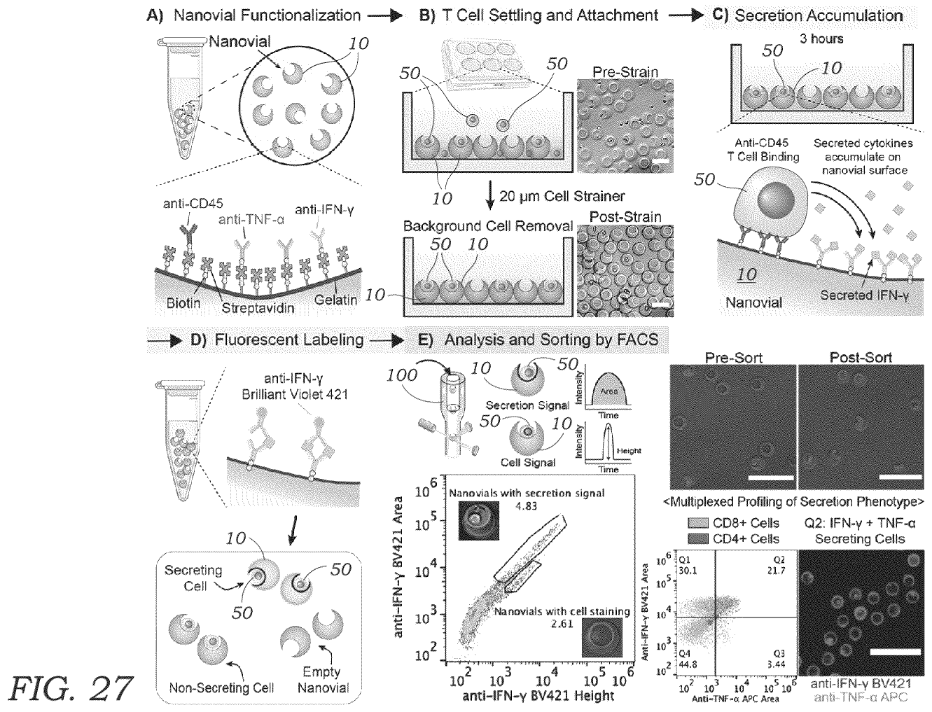

FIG. 27 illustrates the workflow or operations involved in the gelatin-based shaped particle technology used for high-throughput analysis and sorting of individual T cells 50 based on secreted cytokines using a FACS instrument 100. Shaped particles 10 are functionalized with cytokine capture antibodies (anti-IFN-γ, anti-TNF-α, anti-IL-2) and cell binding motifs (anti-CD45). T cells 50 are loaded into the cavities 12 of the shaped particles 10 and, following activation, secrete cytokines which are also captured in the cavity 12 of the shaped particle 10 with minimal crosstalk. Captured cytokines are labeled with a fluorescent detection antibody while cells 50 are labeled with live/dead dyes or additional surface markers. Cytokine binding to shaped particles 10 could be easily distinguished from non-specific cell binding through use of fluorescence peak area and height metrics associated with staining of the larger shaped particles. The particle-based system is used as part of an assay for characterizing four (4) markers simultaneously in live cells 50, two surface markers (CD4 and CD8) and two secreted proteins (IFN-γ and TNF-α or IL-2). Cells 50 are sorted based on the secretion levels using a commercial FACS instrument 100 and remain viable and can be re-cultured after sorting.

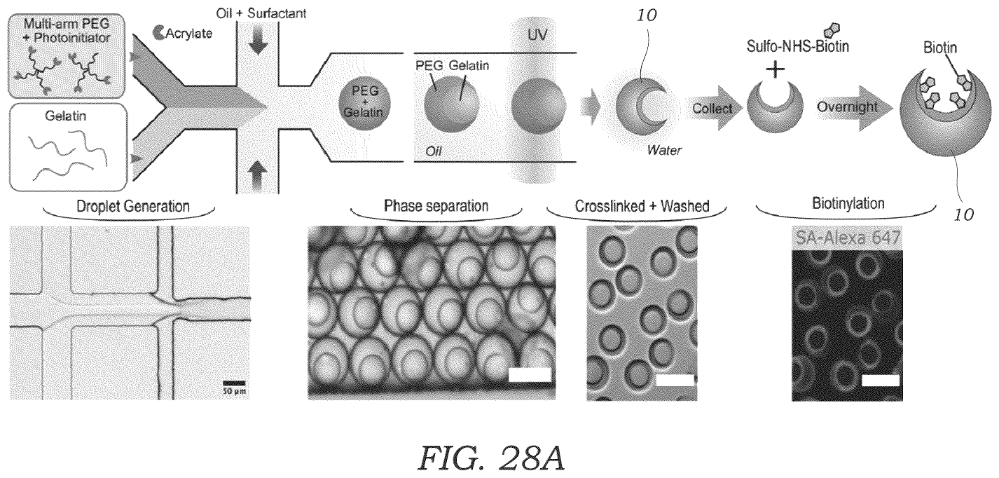

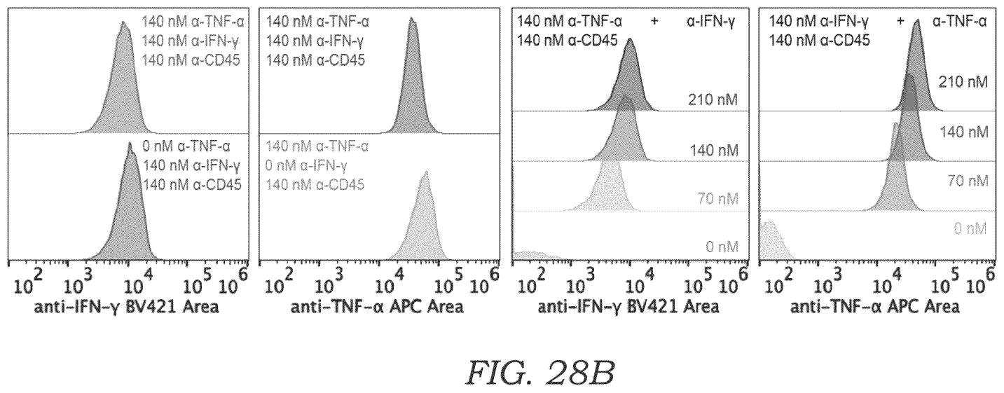

Fabrication and functionalization of shaped particles. To fabricate the shaped particles 10 for the multiplex experiments, an aqueous two-phase system combined with droplet microfluidics and hydrogel chemistry was used. Specifically, a flow-focusing device was used to generate millions of monodisperse polyethylene glycol (PEG)-based shaped particles 10 with the inner cavity selectively coated with biotinylated gelatin (FIG. 28A). To accommodate human T cells 50 with diameters of ˜10 μm uniform shaped particles 10 were fabricated with an average diameter of 35 μm (CV=5.1%) and average cavity of 21.2 μm (CV=7.2%). The shaped particles 10 offer the unique property that this cavity 12 can be functionalized with biotin, reacted with streptavidin and then linked to a number of different biotinylated antibodies to selectively isolate cells 50 based on surface expression and capture secreted molecules. For T cell secretion assays, the shaped particles 10 were decorated with anti-IFN-γ and anti-TNF-α or anti-IL-2 antibodies along with anti-CD45 antibodies to selectively adhere T cells 50 into the cavity 12 of shaped particle 10. In total, 2 or 3 antibodies were linked to the shaped particles 10 through streptavidin-biotin noncovalent interactions. It was found that the optimum ratio between anti-CD45 capture antibodies and cytokine secretion capture antibodies was 1:1 (140 nM each) for single cytokine analysis and 1:1:1 (140 nM each) for multiplexed probing, which allowed for T cell 50 loading as well as signal from recombinant cytokines down to 10 ng/mL (FIG. 28B).

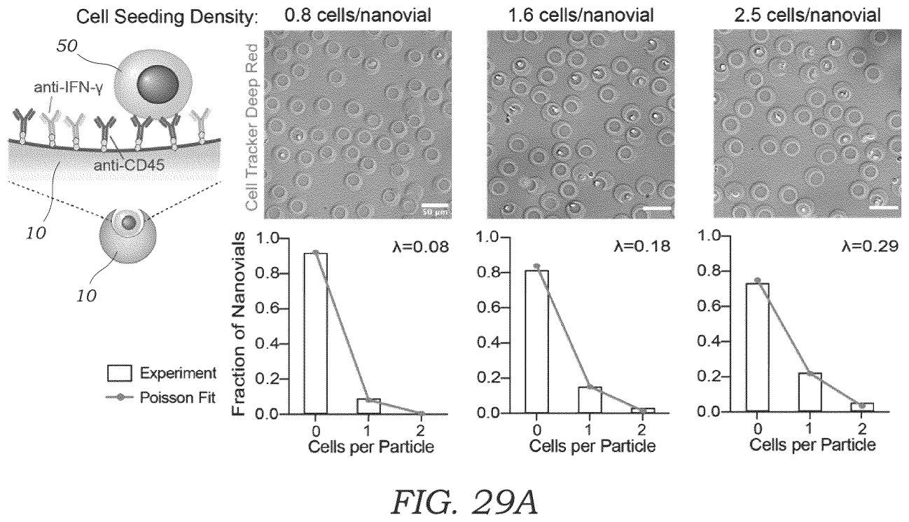

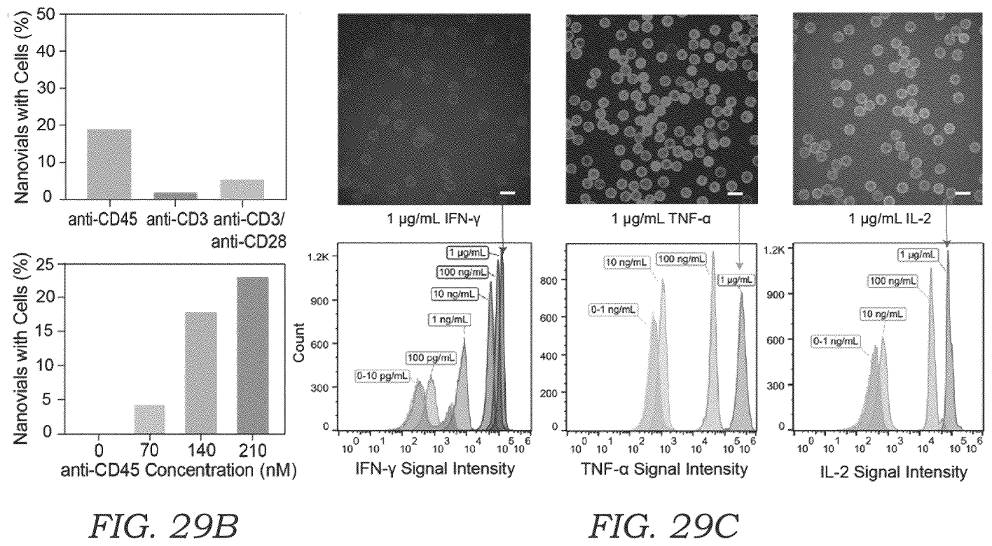

Loading of T cells onto shaped particles. T cells 50 were loaded into the cavities 12 of shaped particles 10 by simply pipetting the cell 50 and particle suspension in a three-dimensional space such as a well-plate or a tube, followed by incubation for one hour to allow cell binding to anti-CD45 antibodies linked to the cavities 12 of the shaped particles 10. Single-cell loading onto shaped particles 10 through binding with CD45 on the surfaces of T cells 50 was achieved by tuning the cell seeding density to shaped particle 10 ratio. When 1.6 cells per shaped particle 10 were seeded in a well plate, 15% of shaped particles 10 contained single cells 50 and only 3.6% had two or more cells 50 (FIG. 29A). When the cell to shaped particle ratio increased, there were more than 10% of shaped particles 10 loaded with more than two cells 50. Depending on the size of the cells 50, the cell seeding density can be adjusted to ensure that most shaped particles 10 have one cell 50 loaded in the cavity 12. Different types of surface protein targets that can serve as cell binding motifs such as anti-CD3 and anti-CD28 were tested, but shaped particles 10 labeled with anti-CD45 had the highest loading efficiency because T cells 50 were initially activated for growth with anti-CD3/anti-CD28 soluble activators prior to being loaded, which reduced CD3 and CD28 availability on the cells 50 (FIG. 29B). Increased anti-CD45 concentration on shaped particles 10 further improved cell binding by nearly 6-fold that shaped particles 10 labeled with 210 nM of anti-CD45 had 23% loading efficiency as compared to 70 nM anti-CD45 labeled shaped particles 10 had 4% loading of cells 50 (FIG. 29B).

Cytokine detection was observed on shaped particles 10 over >3 orders of magnitude dynamic range. The limit of detection of shaped particles 10 was validated by pre-labeling shaped particles 10 with each cytokine capture antibody and incubating with recombinant proteins (IFN-γ, TNF-α, IL-2). Secreted cytokines were labeled with fluorescent detection antibodies and the signal was analyzed by flow cytometry. Cytokine concentrations as low as 1-10 ng/mL were observable above background with at least three orders of magnitude dynamic range, which was found to be sufficient for single-cell analysis (FIG. 29C). When two cytokine capture antibodies were conjugated to the shaped particles 10 (against IFN-γ or TNF-α), the ability to detect each individual cytokine was not significantly affected (FIG. 28B). For the same concentration of recombinant IFN-γ or TNF-α, the average intensity on the shaped particles 10 only changed from 8.4×10³ to 10.8×10³ and 3.7×10⁴ to 5.4×10⁴, respectively.

Measuring single-cell secretion of cytokines on shaped particles using FACS. Single-cell assays targeting three secreted cytokines (IFN-γ, TNF-α, IL-2) were created by labeling streptavidin-coated shaped particles 10 with separate cytokine capture antibodies and anti-CD45. After loading T cells 50 onto shaped particles 10, cells 50 were activated with phorbol 12-myristate 13-acetate (PMA) and ionomycin. Secreted cytokines were accumulated on shaped particles 10 over 3 hours and labeled with fluorescent detection antibodies. From fluorescence microscopy images, two distinct fluorescence patterns were observed, fluorescence spread across the cavity 12 of the shaped particle 10, presumably from secreted cytokines and fluorescence associated with cells 50 on shaped particles 10 (without signal on the shaped particle 10). It was hypothesized that the fluorescent antibody labels binding to cells 50 could be caused by intracellular staining of permeabilized or dead cells, or the presence of membrane bound forms of cytokines on the surface of the cell membrane, especially for TNF-α.

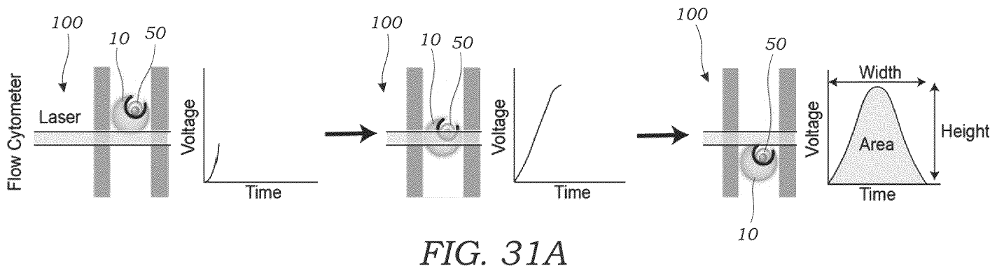

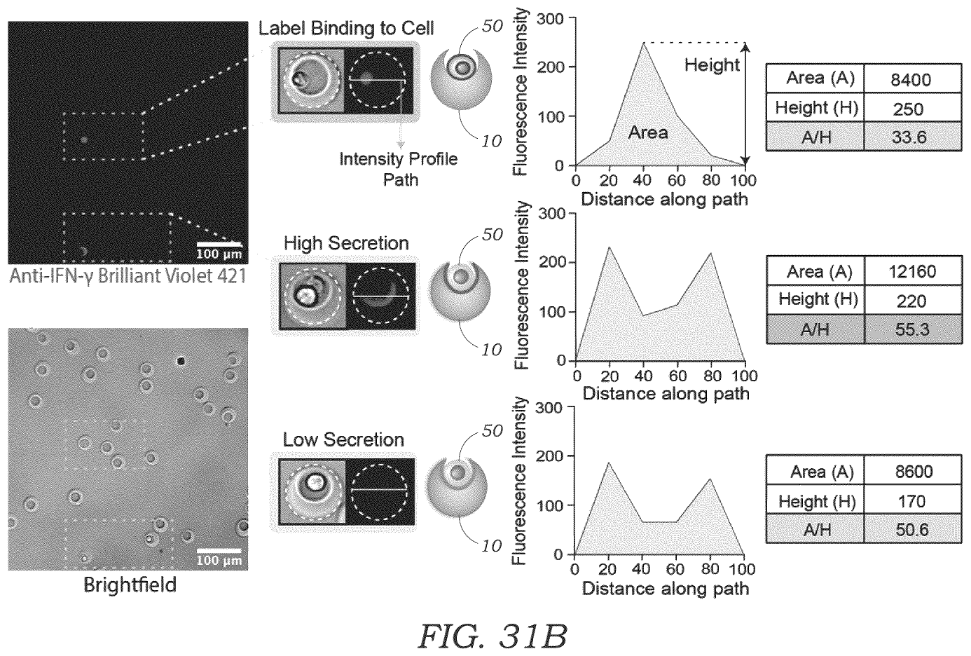

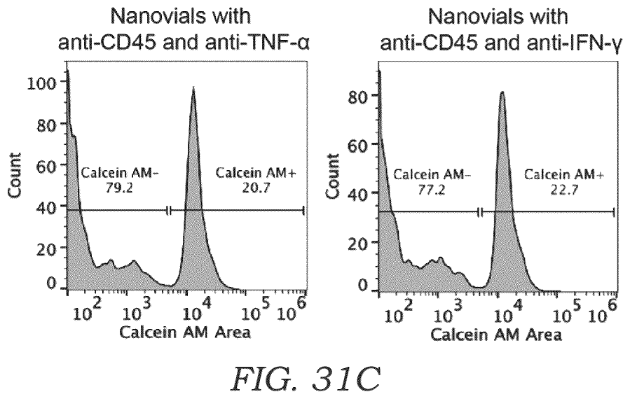

An approach was developed to use the fluorescence peak shape to distinguish between shaped particle 10 staining and cell 50 staining. The spatial fluorescence distribution was examined in microscopy images (FIG. 31B). From fluorescence images of T cells 50 secreting on shaped particles 10 the maximum intensity (height), and area under the intensity curve (area), and the ratio between the area and height were calculated. Shaped particles 10 with spatially spread secretion signal on shaped particles 10 and label bound to cells 50 adhered to shaped particles 10 had similar peak height values, but secretion signal led to a much higher area over height ratio (A/H). This information is important because when a shaped particle 10 passes through the excitation laser spot in flow cytometry, it emits fluorescence light in a time dependent manner generating a pulse that is detected corresponding to this spatial information (FIG. 31A). The height of the pulse is determined by the maximum fluorescence intensity of the shaped particle 10 and the area integrates the intensity emitted over the entire transit event through the laser spot. Accordingly, the shaped particles 10 with spatially-extended secretion signals are expected to produce higher fluorescence area signals for a given fluorescence intensity (height) compared to shaped particles 10 with cells 50 bound to labels.

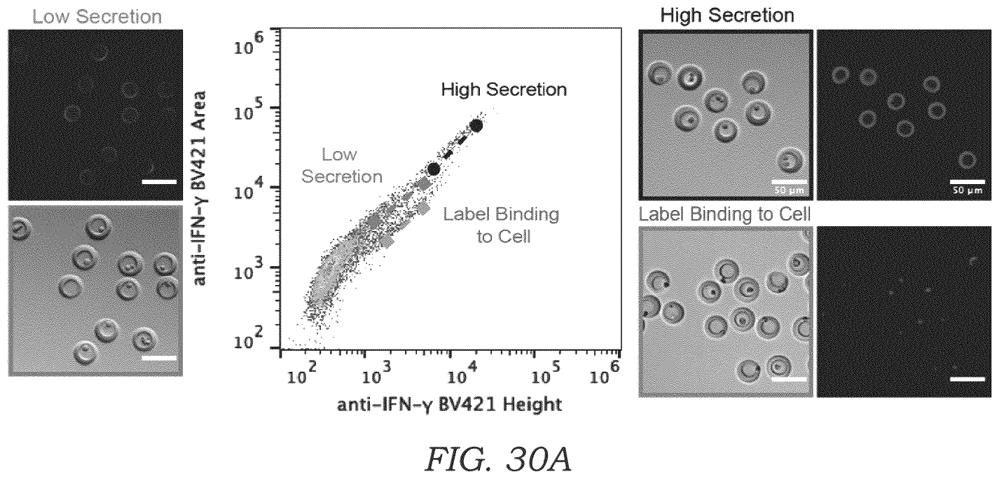

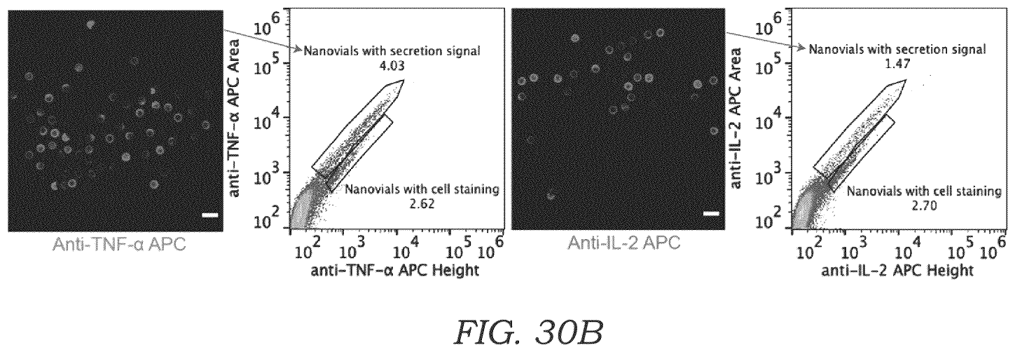

Samples were analyzed based on a combination of fluorescence peak area and peak height signals (area vs. height plot) and observed two populations, where one population had higher area signal as compared to the other population with similar height values. When shaped particles 10 were sorted with larger ratios of area/height (following a line on the log-log plot offset to the upper left), they did correspond to shaped particles 10 with secretion signal, while sorted events in the lower area/height (A/H) region corresponded to shaped particles 10 with label bound to cells 50 (FIG. 30A). Using this area vs. height metric, one is able to sort populations of cells 50 with cytokine secretions only (A/H >3), completely differentiating secretion signal on shaped particles 10 from signal solely from cell surface binding or intracellular staining of permeabilized or dead cells (FIG. 30B). Additional quantitative analysis was performed of how the fluorescence peak shape would change with shaped particle staining compared to cell staining or a combination of both, which provides a guide for differentiating signals from different sources on shaped particles 10 and as a function of different sized shaped particles 10.

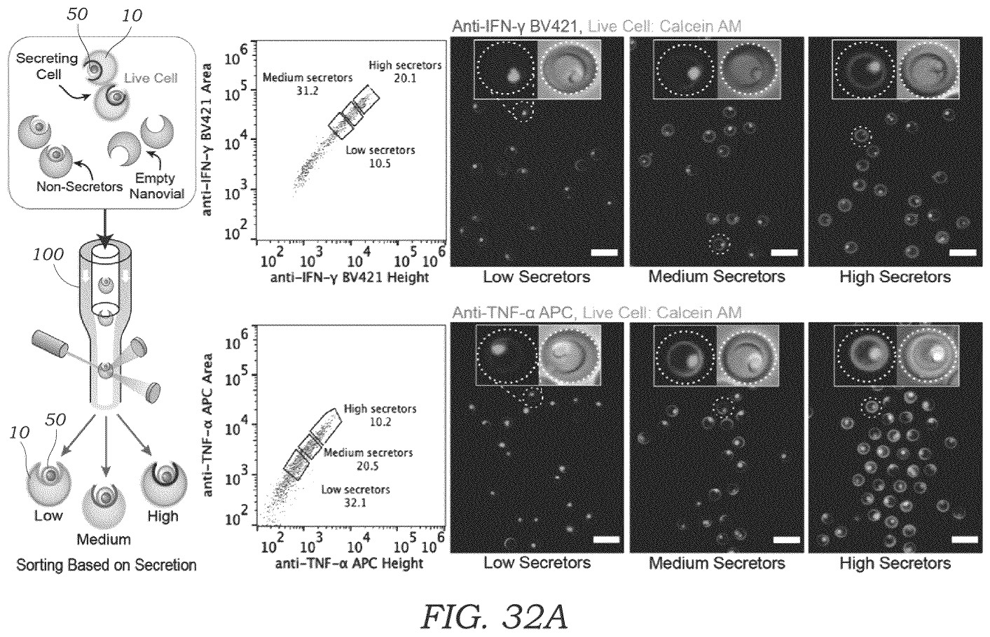

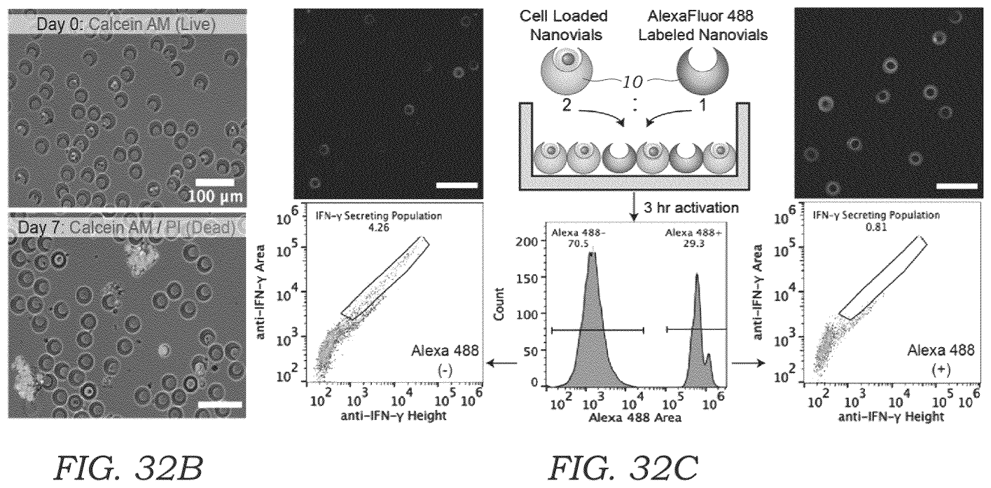

Sorting viable cells based on secretion level. The shaped particle 10 platform was able to simultaneously measure secretions and viability of individual cells 50 on shaped particles 10, improving the selective sorting of functional cells 50. Cells 50 were activated with PMA and Ionomycin and secretions were accumulated for three hours. Cells 50 were stained with calcein AM while secreted cytokines were labeled with fluorescently labeled detection antibodies. Using a FACS instrument 100, the shaped particle population was first gated based on high calcein AM signal and quantified fluorescence secretion signal using a 2D plot of the fluorescence peak area vs. fluorescence peak height (FIG. 32A). It should be noted that when gating on viable cells 50 the number of events with a low area to height ratio was dramatically reduced, suggesting that fluorescent labeling of cells 50 most likely results from intracellular staining of permeabilized or dead cells. The fluorescent secretion signals associated with viable cells 50 for TNF-α and IFN-γ spanned an order of magnitude in both fluorescence peak area and height. Single cells 50 generally secreted higher amounts of IFN-γ, with a distribution that was shifted to higher intensities (>50% of cells 50 were medium or high secretors). Cells 50 secreting TNF-α often had lower secretion signal, with a distribution skewed to lower levels (>50% of cells 50 were low or medium secretors).

Viable cells 50 with different levels of TNF-α and IFN-γ secretion could be recovered following sorting. Sorting was done by first gating on viable cells 50 and then the associated secretion level for TNF-α and IFN-γ. Three regimes were identified in the area vs. height plot to gate populations into low, medium, or high secretors. After sorting, cells 50 remained intact and viable in the shaped particles 10, enabling the regrowth of cells 50 post-sort for further downstream analysis. As shown in FIG. 32B, cells 50 proliferated over 7 days and formed clusters that grew from shaped particles 10 as is expected for T cell culture. In addition, cell-loaded shaped particles 10 were incubated along with fluorescently (AlexaFluor 488) labeled shaped particles 10 without cells 50 to determine the crosstalk between two samples. Secretion signal was analyzed by gating on the green fluorescence signal on shaped particles 10 and found less than 1% of gated shaped particles 10 without cells 50 also appeared in the positive secretion gate during the 3 hour activation period (FIG. 32C), suggesting minimal cross-talk.

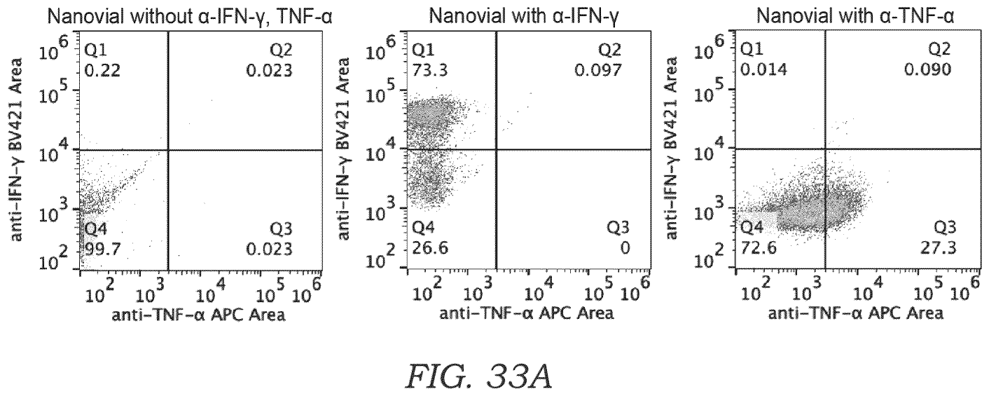

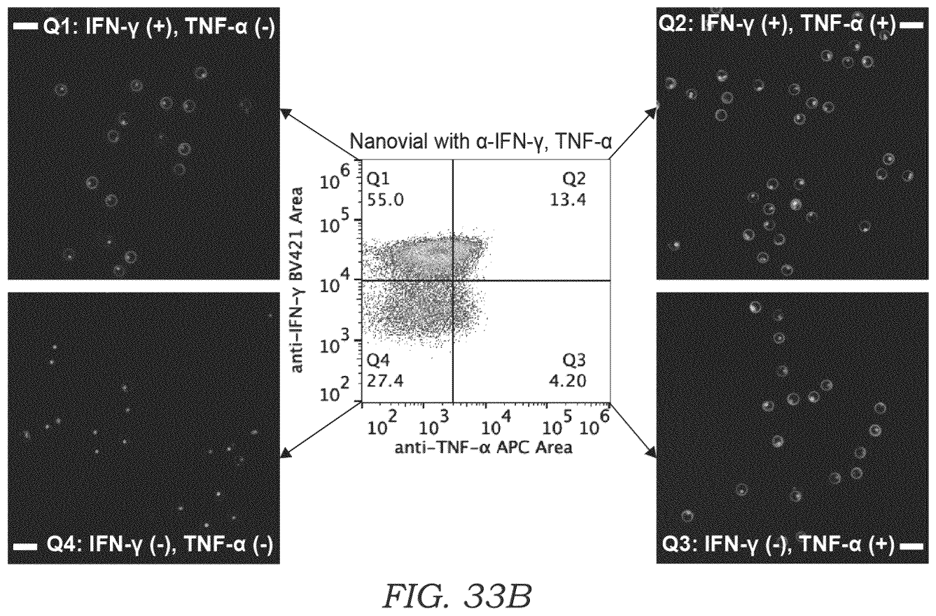

Sorting cells based on a combination of secreted cytokines. By decorating the cavity 12 of shaped particles 10 with two cytokine capture antibodies, multiplexed secretion-based sorting was performed of >1 million cells to select polyfunctional T cells 50 secreting at least two cytokines simultaneously. Shaped particles 10 were pre-labeled with two cytokine capture antibodies against IFN-γ and TNF-α along with anti-CD45 antibodies to capture cells 50. After seeding of cells 50 on the shaped particles 10, cells 50 were activated with PMA and ionomycin and secretions were accumulated over 3 hours. Staining with calcein AM was used to gate out the live cell population only. The secretion signal from shaped particles 10 labeled without any cytokine capture antibody and shaped particles 10 with each cytokine capture antibody alone were analyzed to create a positive threshold gate for IFN-γ, TNF-α or IFN-γ and TNF-α secreting cells (FIGS. 33A-33B). Single-cytokine capture control samples were also stained with each detection antibody individually or with both detection antibodies to determine if there was any crosstalk from the detection antibodies. Individual cytokine signals were found to be independent of the presence of one or more detection antibodies (FIG. 33A).

After the accuracy of the multiplexed system was confirmed and positive secretion gates identified, sorting of T cell populations was performed based on secretion phenotype. It was observed that more than half of T cells 50 secreted only IFN-γ, while 4.2% secreted only TNF-α, and 13.4% secreted both cytokines, and were considered polyfunctional T cells 50 (FIG. 33B). The fraction of polyfunctional T cells 50 was then successfully sorted and recovered, which were observed to have distributed staining of the shaped particle cavity 12 surfaces with both fluorescent anti-cytokine antibodies (FIG. 30B).

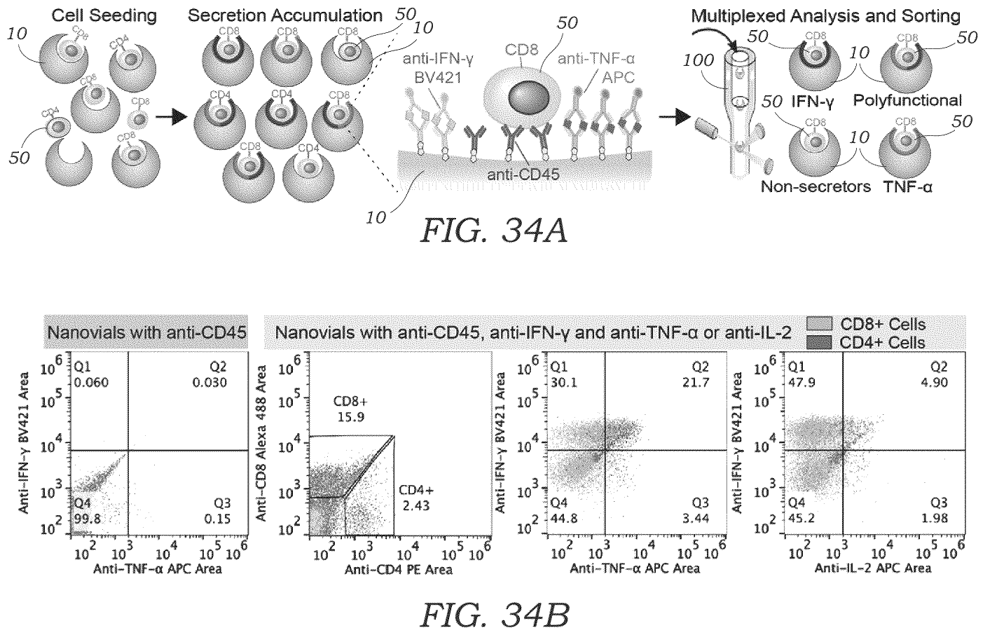

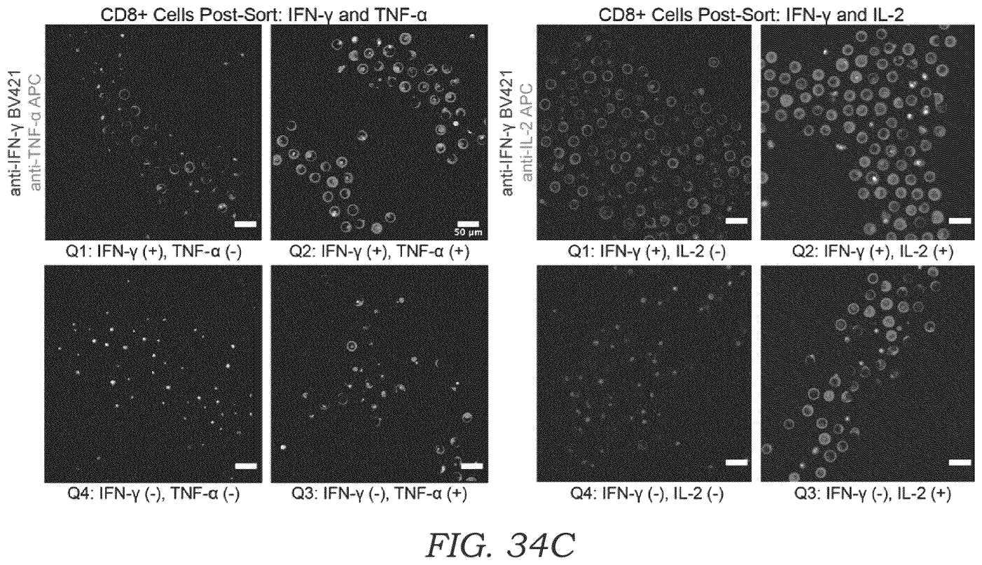

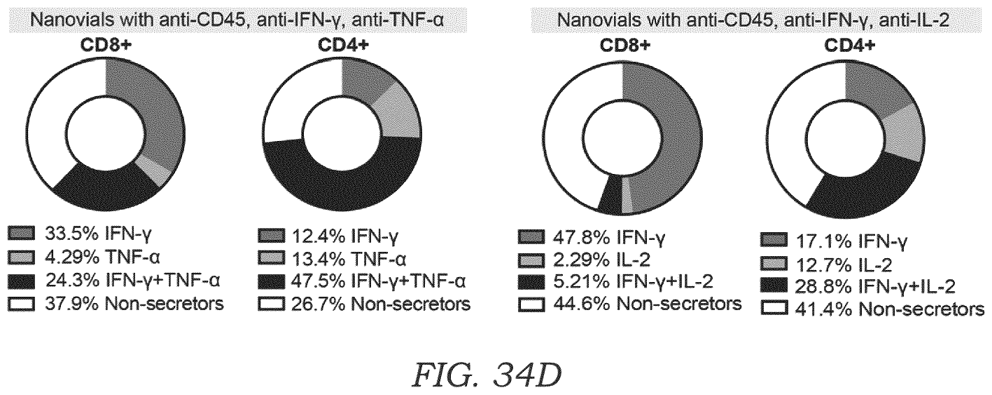

Linking secretions with surface markers from single T cells. The secretion phenotypes identified with the assay were then correlated with surface markers associated with T cell sub-populations. T cells 50 were loaded onto shaped particles 10 conjugated with antibodies against IFN-γ and TNF-α, or antibodies against IFN-γ and IL-2 and anti-CD45 (FIG. 34A). After 3 hours of activation with PMA and ionomycin, cells 50 were stained with fluorescent anti-CD4 and anti-CD8 antibodies and captured cytokines were stained with their respective fluorescent antibodies. The T cell population originally consisted of more CD8+ cells (61.1%) than CD4+ cells (23.5%). When T cells 50 were loaded, 15.9% of shaped particles 10 had CD8+ cells while 2.43% of shaped particles 10 contained CD4+ cells (FIG. 34B). Secreting cells were identified by first creating a gate based on the negative control sample that comprised shaped particles 10 with only anti-CD45 antibodies. populations of cells 50 were sorted based on fluorescence peak areas exceeding the negative control for each individual cytokine as well as combinations of IFN-γ and TNF-α or IL-2 and IFN-γ (FIG. 34C). Cells 50 on shaped particles 10 that fell within the negative control gate were considered non-secretors. When measuring IFN-γ and TNF-α, it was found that the majority of CD8+ cells 50 secreted IFN-γ and only a small fraction of CD8+ cells 50 secreted TNF-α alone (4.3%) (FIG. 34D). About 24% of CD8+ cells 50 were polyfunctional, secreting both IFN-γ and TNF-α simultaneously. On the other hand, CD4+ cells 50 had a larger population that secreted both IFN-γ and TNF-α simultaneously (48%) than CD8+ cells 50. This pattern was consistent when analyzed for IFN-γ and IL-2 secretion. About 29% of CD4+ cells 50 secreted both cytokines while 17% secreted IFN-γ only and 13% secreted IL-2 only. Few CD8+ cells 50 secreted IL-2 alone (2%) or IL-2 and IFN-γ (5%). Although the polyfunctional populations were only a small fraction of the entire cell population, the platform was able to sort and recover them with high viability.

Using a combination of fluorescence peak area and height signals in FACS, one can differentiate secreted cytokines captured on the shaped particle cavity 12 from other fluorescence labeling likely due to intracellular staining of permeabilized or dead cells 50. When using viability dye, this cell labeling could be completely gated out. This analytical feature is particularly important for some secreted proteins as they can also be present on the cell surface. For example, there are both secreted and soluble forms of TNF-α, which would reflect different biological states of a cell 50. TNF-α can be expressed on the surface of activated T cells 50 that exerts various pro-inflammatory functions in a cell-to-cell contact manner, which is distinct from the soluble form of TNF-α, for example, membrane TNF-α was significantly induced on the cell surface of CD8+ T cells from autoimmune disease patients and exerted cytotoxic activity against an anticancer drug resistant cell line. Similarly, for B cells, secreted IgG and the B cell receptor share a common structure. Therefore, the area/height ratio, or other metrics of fluorescence peak width or shape, can be used to distinguish these different states. Images from image cytometry systems or image-activated cell sorting systems would provide the similar ability to distinguish between cell surface staining and staining on shaped particles 10.

### Methods

### List of Reagents and Resources

| REAGENT OR RESOURCE | SOURCE | IDENTIFIER |
|---|---|---|
| Antibodies | | |
| Goat anti-human IgG H&L, | Abcam | ab96911 |
| Dylight ® 488 conjugated | | |
| Goat anti-Human IgG Fc, biotin | Thermo Fisher | A18821 |
| conjugated | | |
| Chemicals, Peptides and | | |
| Recombinant Proteins | | |
| Nanovial fabrication and | | |
| modification | | |
| 4-arm Poly(ethylene glycol) | Sigma-Aldrich | 808474-1G |
| norbornene terminated | | |
| DTT | Sigma-Aldrich | 10197777001 |
| Lithium phenyl-2,4,6- | Sigma-Aldrich | 900889-1G |
| trimethylbenzoylphosphinate | | |
| Biotin PEG thiol | Nanocs | PG2-BNTH-5k |
| RGD peptide (Ac- | Genscript | Custom Order |
| RGDSPGERCG-NH2) | | |
| [SEQ ID NO: 28] | | |
| Dextran from Leuconostoc spp. | Sigma-Aldrich | 31389-25G |
| (40,000) | | |
| Hexane, mixture of isomers | Sigma-Aldrich | 178918-2.5L |
| 1H,1H,2H,2H-Perfluoro-1- | Sigma-Aldrich | 370533-5G |
| octanol | | |
| Thiol-Poly-L-Lysine-Thiol | Nanosoft | 12570-32,000 |
| | Polymers | |
| Fibronectin Human Plasma, | Thermo Fisher | 33016015 |
| Protein | | |
| Pico-Surf ™ (5%(w/w)) in | Sphere Fluidics | CO24 |
| Novec ™ 7500 | | |
| 3M Novec 7500 | Gallade | N17501 |
| | Chemical | |
| Mineral oil, light | Sigma-Aldrich | 330779-1L |
| Cell Culture | | |
| Fetal Bovine Serum US Certified | Invitrogen | 16000044 |
| Penicillin-Streptomycin (10,000 | Invitrogen | 15140122 |
| U/mL) | | |
| DMEM, high glucose, pyruvate | Invitrogen | 11995065 |
| Insulin, Human recombinant | Sigma-Aldrich | 91077C-100MG |
| Trace Elements A | Thermo Fisher | MT99182CI |
| Trace Elements B | Thermo Fisher | MT99175CI |
| Methotrexate hydrate | Sigma-Aldrich | M8407-100MG |
| Gibco ™ ExpiCHO ™ Expression | Fisher | A2910001 |
| Medium | Scientific | |
| ExpiCHO-S cells | Fisher | A29127 |
| | Scientific | |
| HEK293 cells | ATCC | CRL-1573TM |
| Secretion Assay | | |
| Streptavidin (5 mg/ml) | Thermo Fisher | 434302 |
| Streptavidin, Alexa Fluor ™ | Fisher | S11226A |
| conjugated | Scientific | |
| CellTracker Deep Red Dye | Thermo Fisher | C34565 |
| DPBS (Ca+, Mg+) | Invitrogen | 14040133 |
| BSA Bovine Serum Albumin | GeminiBio | 700-102P |
| (BSA), Fraction V-Low- | | |
| Endotoxin Grade | | |
| Pluronic ™ F-127 | Sigma | P2443-250G |
| Reversible Strainers (37 um) | Stemcell | 27215 |
| | Technologies | |
| Single-Cell RT-PCR | | |
| RNasin ® Plus Ribonuclease | Promega | N2611 |
| Inhibitor | | |
| SuperScript III Reverse | Thermo Fisher | 18080 |
| Transcriptase | | |
| Phusion High Fidelity DNA | Thermo Fisher | F530S |
| Polymerase | | |
| Experimental Models: Cell Lines | | |
| Hamster: CHO-K1 (CCL61) | ATCC | CCL61 |
| Hamster: CHO DP-12 (CRL- | ATCC | CRL-12445 |
| 12445) | | |
| Human: HEK293 cells | ATCC | CRL-1573 ™ |
| Hamster: ExpiCHO-S cells | Fisher | A29127 |
| | Scientific | |
| Oligonucleotides | | |
| Heavy chain 1^{st} PCR forward | | AGGAACTGCAGGTGTCC |
| primer: 1mFH_I | | [SEQ ID NO: 1] |
| Heavy chain 1^{st} PCR forward | | CAGCTACAGGTGTCCACTCC |
| primer: 1mFH_II | | [SEQ ID NO: 2] |
| Heavy chain 1^{st} PCR forward | | TGGCAGCARCAGCTACAGG |
| primer: 1mFH_III | | [SEQ ID NO: 3] |
| Heavy chain 1^{st} PCR forward | | CTGCCTGGTGACATTCCCA |
| primer: 1mFH_IV | | [SEQ ID NO: 4] |
| Heavy chain 1^{st} PCR forward | | CCAAGCTGTGTCCTGTC |
| primer: 1mFH_V | | [SEQ ID NO: 5] |
| Heavy chain 1^{st} PCR forward | | TTTTAAAAGGTGTCCAGKGT |
| primer: 1mFH_VI | | [SEQ ID NO: 6] |
| Heavy chain 1^{st} PCR forward | | CCTGTCAGTAACTRCAGGTGTCC |
| primer: 1mFH_VII | | [SEQ ID NO: 7] |
| Heavy chain 1^{st} PCR forward | | TTTTAAAAGGGGTCCAGTGT |
| primer: 1mFH VIII | | [SEQ ID NO: 8] |
| Heavy chain 1^{st} PCR forward | | CGTTCCTGGTATCCTGTCT |
| primer: 1mFH_IX | | [SEQ ID NO: 9] |
| Heavy chain 1^{st} PCR forward | | ATGAAGTTGTGGYTRAACTGG |
| primer: 1mFH_X | | [SEQ ID NO: 10] |
| Heavy chain 1^{st} PCR forward | | TGTTGGGGCTKAAGTGGG |
| primer: 1mFH_XI | | [SEQ ID NO: 11] |
| Heavy chain 1^{st} PCR reverse | | AGAAGGTGTGCACACCGCTGGAC |
| primer: 1mRG* (Gamma) | | [SEQ ID NO: 12] |
| Heavy chain 2^{nd} PCR forward | | GGGAATTCGAGGTGCAGCTGCAGGAGTCTGG |
| primer: 2mFG* | | [SEQ ID NO: 13] |
| Heavy chain 2^{nd} PCR reverse | | GCTCAGGGAARTAGCCCTTGAC |
| primer: | | [SEQ ID NO: 14] |
| K chain 1^{st} PCR forward primer: | | RGTGCAGATTTTCAGCTTCCTGCT |
| 1mFK_I | | [SEQ ID NO: 15] |
| K chain 1^{st} PCR forward primer: | | TGGACATGAGGGCYCCTGCTCAGT |
| 1mFK_II | | [SEQ ID NO: 16] |
| K chain 1^{st} PCR forward primer: | | CTSTGGTTGTCTGGTGTTGAYGGA |
| 1mFK_III | | [SEQ ID NO: 17] |
| K chain 1^{st} PCR forward primer: | | GTTGCTGCTGCTGTGGCTTACA |
| 1mFK_IV | | [SEQ ID NO: 18] |
| K chain 1^{st} PCR forward primer: | | GTATCTGGTACCTGTGG |
| 1mFK_V | | [SEQ ID NO: 19] |
| K chain 1^{st} PCR forward primer: | | TGCCTGTTAGGCTGTTGGTGCT |
| 1mFK_VI | | [SEQ ID NO: 20] |
| K chain 1^{st} PCR forward primer: | | GCTCAGTTCCTTGGTCTCCTGTTGC |
| 1mFK_VII | | [SEQ ID NO: 21] |
| K chain 1^{st} PCR forward primer: | | TGGGTGCTGCTGCTCTGGGT |
| 1mFK_VIII | | [SEQ ID NO: 22] |
| K chain 1^{st} PCR forward primer: | | CAGTTCCTGTTTCTGTTARTGCTCTGG |
| 1mFK_IX | | [SEQ ID NO: 23] |
| K chain 1^{st} PCR forward primer: | | TGCTCTGGTTATATGGTGCTGATGGG |
| 1mFK_X | | [SEQ ID NO: 24] |
| K chain 1^{st} PCR reverse primer: | | ACTGAGGCACCTCCAGATGTT |
| 1mRK* | | [SEQ ID NO: 25] |
| K chain 2^{nd} PCR forward primer: | | GAYATTGTGMTSACMCARWCTMCA |
| 2mFK* | | [SEQ ID NO: 26] |
| K chain 2^{nd} PCR reverse primer: | | TGGGAAGATGGATACAGTT |
| 2mRK* | | [SEQ ID NO: 27] |
| Software and Algorithms | | |
| Image J | NIH | https://imagej.nih.gov/ij |
| Matlab | Mathworks | Version 2016b |
| FlowJo | FlowJo, LLC | |

Fabrication of Shaped Particles (non-multiplexed). Shaped particles 10 were fabricated using a standard PDMS microfluidic flow focusing droplet generator (FIGS. 12A and 12B) an example of which may be found in U.S. Patent Application Publication No. 2021/0268465, which is incorporated herein by reference. A PEG phase comprised of 28.9% w/w 4-arm PEG-Norbornene (Sigma), 3% w/w LAP (Lithium phenyl-2,4,6-trimethylbenzoylphosphinate,sigma), and 1 mg/ml Biotin-PEG-thiol (5000 MW, Nanocs) in Phosphate Buffered Saline (PBS, pH7.2) was co-injected with a dextran phase comprised of 11% w/w 40 kDa dextran (Sigma), 1.3% w/w DTT (dithiothreitol, Sigma), and 5 mM RGD peptide (Ac-RGDSPGERCG-NH2 [SEQ ID NO:28], Genscript) in PBS at a rates of 0.5-5 μL/min, depending on the shaped particle 10 size, using syringe pumps (Harvard Apparatus PHD 2000). An oil phase comprised of Novec™ 7500 (3M) and 0.25% w/w Pico-Surf™ (Sphere Fluidics) was injected at a rate of 10-42 μL/min to partition the aqueous phases into monodisperse water and oil droplets. PEG and dextran polymers phase separated on chip after approximately 5 seconds (FIG. 12C showing phase separation). The PEG phase was crosslinked with focused UV light through a DAPI filter set and microscope objective (Nikon, Eclipse Ti-S) near the outlet region of the microfluidic device. Crosslinked shaped particles 12 were collected and oil and dextran were removed using a series of washing steps (FIG. 12D). Briefly, excess oil was removed by pipetting and a layer of PBS was added on top of the remaining emulsions. A solution of 20% v/v perfluoro octanol (PFO, Sigma) in Novec™ 7500 was then added to destabilize the emulsions and transfer the shaped particles 10 to the PBS phase. Excess oil was removed and samples were washed 2× with Novec™ 7500 to remove remaining surfactant. Novec™ 7500 was removed by pipetting and residual oil was removed by washing 2-3× with hexane (Sigma). Samples were then washed 3× with PBS to remove dextran from the system. For cell experiments the shaped particles 10 were sterilized by incubating in 70% ethanol overnight. Shaped particles 10 were then washed 5× with a storage solution comprised of PBS+0.1% Pluronic F-127 (Sigma)+1% penicillin/streptomycin (Invitrogen) and stored in a conical tube at 4° C.

Fabrication of Shaped Particles (multiplexed). Shaped particles 10 were fabricated using a three inlet flow-focusing microfluidic droplet generator formed from polydimethylsiloxane as discussed above. In respective inlets, PEG pre-polymer, gelatin and oil phases were infused at flow rates of 1.5 μl/min, 1.5 μl/min, and 15 μl/min respectively. The PEG pre-polymer phase comprised 27.5% w/v 5 kDa 4-arm PEG acrylate (Advanced BioChemicals) with 4% w/v lithium phenyl2,4,6-trimethylbenzoylphosphinate (LAP, Sigma) in phosphate buffered saline (PBS, pH 7.2). The gelatin phase comprised 20% w/v cold water fish gelatin (Sigma) in deionized water. The oil phase comprised Novec™ 7500 (3M) with 0.5% v/v Pico-surf (Sphere Fluidics). Oil partitioned the aqueous phases into monodisperse water-in-oil droplets and PEG-gelatin polymers phase separated after approximately 5 seconds, followed by cross-linking with focused UV light through a DAPI filter set and microscope objective (Nikon, Eclipse Ti-S) near the outlet region of the microfluidic device. Polymerized shaped particles 10 were collected in a conical tube and any unreacted phases including oil were removed through a series of washing steps as previously described(16, 17). Biotinylation of the gelatin-layer formed in the particle cavity 12 was conducted by incubating shaped particles 10 with 10 mM Sulfo-NHS-Biotin (APExBIO) overnight at 4° C. Shaped particles 10 were then washed in Washing Buffer consisting of 0.05% Pluronic F-127 (Sigma), 1% 1× antibiotic-antimycotic (Thermo Fisher), and 0.5% bovine serum albumin (BSA, Sigma) in PBS and sterilized in 70% ethanol overnight. Sterile shaped particles 10 were stored at 4° C. in Washing Buffer.

### Cell Culture

CHO K1 and CHO DP-12 cells. All cells were cultured in incubators at 37° C. and 5% CO₂. CHO K1 cells (ATCC CCL61) were cultured in F12 base media (Invitrogen) supplemented with 10% fetal bovine serum (FBS, Invitrogen) and 1% penicillin/streptomycin (Invitrogen). CHO DP-12 cells (ATCC CRL-12445) were maintained according to manufacturer's specifications. Cell culture media was comprised of DMEM (Invitrogen) supplemented with 10% FBS, 1% penicillin/streptomycin, 0.002 mg/ml recombinant human insulin (Sigma), 0.1% Trace Elements A (Fisher Scientific), 0.1% Trace Elements B (Fisher Scientific), and 200 nM Methotrexate (MTX, SIGMA).

ExpiCHO cells. ExpiCHO cells (Fisher Scientific A29127) were cultured in ExpiCHO™ Expression Medium (Fisher Scientific) on an orbital shaker in a 37° C. incubator with ≥80% relative humidity and 8% CO₂. The shake speed was set to 120 rpm with 25 mm shaking diameter. Cells were seeded at 0.2×10⁶ viable cells/mL and subcultured when the cell density reached 4×10⁶-6×10⁶ viable cells/mL.

HEK293 cells. HEK293 cells (ATCC CRL-1573™) were cultured in incubators at 37° C. and 5% CO₂ and the media consisted of DMEM, high glucose, pyruvate media (Invitrogen) supplemented with 10% FBS (Invitrogen). Cells were seeded at 2×10⁴ viable cells/cm² and subcultured when concentration reached 6×10⁴ cells/cm².

Particle seeding and cell seeding characterization. Stock shaped particles 10 (nanovials) were first concentrated in a conical tube by centrifugation and supernatant was aspirated. Shaped particles 10 were then fluorescently labeled by diluting at a 1:1 ratio with PBS containing 10 μg/ml of Alexa Fluor 568 streptavidin (Fisher Scientific) and incubating for 10 min. Shaped particles 10 were washed 3× with PBS and then dispersed in a well plate at a concentration of 7.5 μL of concentrated particles per cm of well surface area. Shaped particles 10 were then allowed to settle for 10 min. CHO cells 50 pre-stained with Hoechst were then seeded at a range of concentrations (31-190 cells/mm²) into the well by carefully pipetting evenly across the well area. Cells 50 were allowed to settle for 15 min and then imaged using a fluorescent microscope. Loading statistics was calculated using custom image analysis algorithms in MATLAB. Shaped particles 10 were first identified using the particle fluorescence channel and cell number was calculated by counting nuclei number within each particle cavity. The amount of shaped particles 10 and cells 50 to seed for target loading fraction are estimated as follows:

$${N}_{n a n o v i a l s} ( {D}_{out} , {SA}_{wellplate} ) = \frac{{SA}_{wellplate} [ {cm}^{2} ] \times 1 {0}^{8}}{{SA}_{N a n o v i a l} [ μ {m}^{2} ]} \times P {F}_{2 D} \qquad ( 1 )$$

Where N is number of shaped particles 10 or nanovials to cover the well plate bottom, PF_{2D} is the packing fraction of the shaped particles 10 or nanovials on a flat surface (˜0.8-0.9) and the projected surface area of each shaped particle 10 or nanovial is:

$$S {A}_{N a n o v i a l s} = {π ( \frac{{D}_{o u t} [ μ m ]}{2} )}^{2} \qquad ( 2 )$$

The volume of concentrated shaped particles 10 or nanovials to add to each well is calculated as:

$${V}_{n a n o v i a l} [ μ L ] ( {D}_{out} , {N}_{n a n o v i a l} ) = \frac{4}{3} {π ( \frac{{D}_{o u t} [ μ m ]}{2} )}^{3} \times \frac{{N}_{n a n o v i a l s}}{P {F}_{3 D}} \times 1 {0}^{9} \qquad ( 3 )$$

Where PF_{3D} is the expected packing fraction of the shaped particles 10 or nanovials which is estimated to be within the range of 0.7-0.8. Based on the number of shaped particles 10 or nanovials seeded for a set well plate size, the expected number of cells to seed for a target loading fraction is estimated as:

$${N}_{cells} ( {D}_{out} , {D}_{cavity} , {N}_{nanovials} , {λ}_{target} ) = {N}_{nanovials} \times λ \times {( \frac{{D}_{o u t}}{{D}_{cavity}} )}^{2} \times \frac{1}{{PF}_{2 D}} \qquad ( 4 )$$

### Cell Binding Characterization

### ExpiCHO Cell Binding

PLL modification on particle surface. In order to modify the particle surface with Poly-L-Lysine, 100 μl of 55 μm shaped particles 10 were suspended in 1 ml solution consisting of 1 mg/ml PLL (Thiol-Poly-L-Lysine-Thiol, 32 kDa, Nanosoft Polymers) and 0.2% w/w LAP (Lithium phenyl-2,4,6-trimethylbenzoylphosphinate, Sigma). The particle suspension was transferred to a glass vial pre-coated with Sigmacote (Sigma SL2-25ML) and the vial was placed on top of a mini stir plate (IKA Lab Disc IKAMAG Magnetic Stirrer) with a micro stir bar in it. With continuous mixing, the particle suspension was exposed to UV for 60 seconds at the power of 2.9 mW/cm². The particle suspension was retrieved into a 1.5 ml Eppendorf tube and washed with 0.05% Pluronic buffer three times.

Fibronectin adhesion to PLL particles. 25 μl of PLL conjugated-shaped particles 10 were incubated in 500 μl of 10 μg/ml or 500 μg/ml fibronectin solution for one hour at room temperature and washed with 0.05% Pluronic buffer three times.

ExpiCHO cell loading assay. In each well of a 24 well-plate, 1 ml of ExpiCHO expression media with 7.84 μl of PLL or PLL+Fibronectin modified particles and 79,000 ExpiCHO cells were added. Particle and cell suspension was mixed 10 times using a 1000 μl pipette. The plate was incubated on a rocker in 37° C. incubator for 30 minutes, and then incubated for another 2.5 hours on a steady state in the incubator. Particle and cell suspension in each well was strained using 37 μm strainer (Stemcell Technologies) and shaped particles 10 were recovered in fresh and pre-warmed ExpiCHO expression media. Recovered shaped particles 10 were imaged using fluorescent microscope.

HEK293 cell loading assay. PLL and PLL+Fibronectin particles were prepared by the same method mentioned in the ExpiCHO cell binding section above. In each well of a 24 well-plate, 1 ml of DMEM base media (without FBS) with 7.84 μl of PLL or PLL+Fibronectin modified particles and 79,000 HEK293 cells were added. Particle and cell suspension was mixed 10 times using a 1000 μl pipette. The plate was incubated on a rocker in 37° C. incubator for 30 minutes, and then incubated for another 2.5 hours on a steady state in the incubator. Particle and cell suspension in each well was strained using 37 μm strainer (Stemcell Technologies) and shaped particles 10 were recovered in fresh and pre-warmed DMEM media with 10% FBS. Recovered shaped particles 10 were imaged using fluorescent microscope.

B cell binding assay. PLL+Fibronectin shaped particles 10 were prepared as mentioned in ExpiCHO cell binding section above. To test capture with anti-surface marker antibodies, particles were first incubated for 10 min with μg streptavidin per μL particle solution. Shaped particles 10 were then washed 3× and incubated with a solution containing 2 ng anti-CD45 (aCD45), anti-CD19 (aCD19), or 1:1 mix of aCD45+aCD19 per μL particle solution at 60 μg total antibody concentration. Shaped particles 10 were then washed 3×. 8 μL of particles were added to each well of a 24 well plate in 2 mL washing buffer, followed by addition of 100,000 cells 50. The cells 50 were allowed to settle into particle voids or cavities 12 and adhere for 2 hours. The shaped particles 10 were then strained with a 20 μm cell strainer to remove unattached cells 50, and analyzed/sorted by fluorescence microscopy or a Sony SH800 FACS instrument 100.

Dropicle formation and characterization. 85 μm shaped particles 10 were suspended in DMEM base media (Invitrogen) and then concentrated by centrifuging at 300 g for 2-3 minutes and aspirating supernatant. An oil phase comprised of Novec™ 7500 and 2% w/w Pico-Surf™ was added to the particle suspension at approximately 2× the remaining volume. The sample was then vigorously pipetted for 30-60 s (˜100 pipettes) using a 200 μL micropipette (Eppendorf). The resulting water-in-oil emulsion was then carefully transferred into a PDMS reservoir by pipetting and imaged. Size distribution characterization was then performed using custom image analysis algorithms in MATLAB. For the fluorescence images shown in FIG. 2B, shaped particles 10 were first labeled with Alexa Fluor 568 streptavidin and the DMEM was replaced with PBS containing 2 mg/ml fluorescein isothiocyanate-dextran (500 k MW, Sigma). For 35 μm shaped particles 10 the same procedure was used except the shaped particles 10 were first diluted 10-15× to reduce fraction of aggregates formed.

Cell viability characterization. 30 μL of concentrated shaped particles 10 and 48 k CHO DP-12 cells 50 were suspended in media and sequentially seeded into individual wells of a 12-well plate. A separate well plate control sample containing no shaped particles 10 was prepared in parallel. Cells 50 were allowed to adhere for 4 hours. Samples containing both the cells 50 and shaped particles 10 were transferred into a 15 ml conical tube, exchanged with fresh media and then concentrated via centrifugation. 100 μL of Novec™+2% w/w Pico-Surf™ was added to the concentrated sample and pipetted for 30 s to encapsulate the shaped particles 10 and associated cells 50 into dropicles. 150 μL of light mineral oil was added on top of the samples to mitigate evaporation during incubation. Samples were then incubated for 2, 12, and 24 hours in an incubator at 37° C. and 5% CO₂. To recover cells 50 back into an aqueous phase excess oil was first removed via pipetting and several ml of media was added on top of the emulsions. To destabilize the droplets 50 μL of 20% v/v PFO in Novec™ 7500 was then pipetted on top of the emulsion layer and the sample was gently agitated. After 5 min most of the droplets were merged and shaped particles 10 and associated cells 50 transferred into the bulk media phase. Optionally, samples can be centrifuged for 15-30 s at 200 g to coalesce remaining droplets. The suspension of shaped particles 10 and cells 50 were then transferred by pipetting into a separate conical tube. Samples were washed with PBS and then sequentially stained with calcein AM and propidium iodide. Particle samples were transferred back into a well plate, imaged, and then analyzed in MATLAB to determine cell viability statistics.

CHO cell secretion assay. To identify cells 50 during downstream analysis CHO DP-12 cells were first stained with CellTracker™ Blue CMAC Dye (Thermo Fisher). RGD coated shaped particles 10 and cells 50 were seeded into a 12-well plate as described above and then incubated at 37° C. for 2 hours to allow cells 50 to adhere to the shaped particles 10. The shaped particles 10 were then recovered by tilting the well plate and transferring by pipetting. In order to remove unattached cells 50 from the background, samples were strained using a 37 μm reversible cell strainer (STEMCELL Technologies) and then shaped particles 10 were recovered by flipping the cell strainer and washing with a washing buffer comprised of PBS (Ca²⁺, Mg²⁺), 0.5% BSA (GeminiBio), 1% penicillin/streptomycin and 0.05% Pluronic F-127. To reduce sample loss all conical tubes and pipette tips were first precoated with washing buffer prior to handling particle containing solutions. After recovering from the well plate, samples were washed 2× by centrifuging shaped particles 10 and associated cells 50 at 300 rpm for 3 min, aspirating, and then resuspending in washing buffer. Particles were then labeled with streptavidin (Thermo Fisher, 434302) by adding 0.415 ng per μL of concentrated particle solution and incubating for 10 min. Samples were then washed 3× with washing buffer and resuspended. Particles were then labeled with Biotin Anti-FC (Thermo Fisher, A18821) by adding 75 μg of the antibodies per μL of concentrated particle solution and incubating for 10 min. After incubation, samples were washed and resuspended in CHO DP-12 media. After re-concentrating, samples were compartmentalized by pipetting with oil and surfactant as described above to create dropicles. Samples were then incubated for a range of times 0, 1, 2, 4, and 8 hours to allow cells 50 to secrete and to facilitate capture of secreted antibodies onto the associated particle matrix via Anti-FC sites. After the incubation period, shaped particles 10 and associated cells 50 were transferred back into media by breaking the emulsions (see live dead section). Samples were then washed and captured secretions were stained with Goat anti-human IgG H&L (Dylight® 488, Abcam ab96911) at a final concentration of 30 μg per μL of initial concentrated particle solution. After 30 min of staining, samples were then washed 5× with a solution of PBS (Ca²⁺, Mg²⁺), 2% FBS, 1% penicillin/streptomycin and 0.05% Pluronic F-127 and optionally stained with propidium iodide. Samples were then imaged in both brightfield and fluorescence channels in a well plate. To characterize secretion amount per particle a custom MATLAB algorithm was used to identify shaped particles 10 in brightfield and then count the number of cells 50, check for the presence of dead stain, and integrate the total secretion label fluorescence intensity for each particle.

Secretion cross-talk analysis experiment. To analyze potential cross-talk in the single cell secretion system, samples were prepared as described in the cell secretion assay section with several modifications to the protocol. Two sets of samples were prepared, a control sample that was incubated in bulk solution and one incubated after dropicle formation. Prior to the incubation step a separate suspension of shaped particles 10 containing no cells 50 and tagged with Alexa Fluor 647 streptavidin were mixed into the samples. This was done in order to ensure signal on empty shaped particles 10 that was measured did not arise from cells 50 that may have detached from the shaped particles 10 during various steps of the assay. Samples were washed and affinity capture agents 16 were then added. The bulk samples were then left to incubate in media while the dropicle samples was emulsified in oil via pipetting. After incubating for 15 hours samples were recovered and washed with washing buffer, stained, and imaged as described in the cell secretion assay section. The amount of cross talk was determined by comparing secretion staining intensity on shaped particles 10 with cells 50 with the intensity on the control particles.

Secretion based sorting control experiment. Samples were prepared as described in the cell secretion assay with the following modifications. CHO DP-12 cells 50 and CHO K1 cells 50 were prelabeled with CellTracker™ Deep Red and CellTracker™ Blue (Thermo Fisher). After labelling, cells 50 were mixed together at various ratios (1:5, 1:100, 1:1000) and loaded into shaped particles 10 (Seeding density ˜84/mm², lambda ˜0.1. All remaining secretion assay steps were as previously described. After labelling secretions on samples with fluorescently labeled secondary antibodies, samples were sorted using a FACS instrument 100 (BioSorter, Unionbioetrica). Samples were excited using both 488 nm and 561 nm lasers. Events were triggered based on particle absorbance from the 561 nm laser. Single particle events were gated based on time of flight. Shaped particles 10 with secretion signal were then sorted by thresholding the peak intensity height collected through a 543/22 nm filter. Samples were sorted directly into a 96-well plate and imaged with a fluorescence microscope.

Enrichment of high producing CHO cells. Samples were prepared as described in the cell secretion assay section. After labelling secretions, a fraction of each sample was kept and imaged using fluorescence microscopy. Remaining samples were then sorted using a FACS instrument 100 (On-Chip Sort, On Chip Biotechnologies). Samples were excited with both a 488 nm and 637 nm laser. Particle events were screened based on the forward and side scatter. Shaped particles 10 positive for both cells and secretion signal were gated based on peak fluorescence height collected through a 676/37 nm emission filter and a 543/22 nm emission filter respectively. Two sub-populations were sorted for each sample: (1) shaped particles 10 with cells 50 and positive secretion signal, (2) shaped particles 10 with cells 50 and the top 20% of positive secretion signal. Collected samples were plated and expanded for >10 days directly from the shaped particles 10. After expansion, cells 50 were released from the shaped particles 10 via trypsinization (e.g., incubation with trypsin EDTA to break down integrin mediated adhesion) and the shaped particles 10 were removed from solution by using a 40 μm cell strainer. To quantify antibody production of the isolated sub-populations, 30,000 cells 50 from the expanded sub-populations as well as un-sorted control samples were plated into a 48-well plate. After cells 50 attached the samples were washed, replaced with 400 μL of fresh media, and incubated for 6 hours. Supernatant was then collected and total human IgG amount was measured using ELISA (IgG (Total) Human ELISA Kit, Invitrogen, BMS2091). Production rate was calculated based off the measured IgG concentration, incubation time, and initial number of cells seeded.

### Mouse Immunization and B Cell Isolation and Culture.

All experiments involving animals, animal cells 50, or tissues were performed in accordance with the Chancellor's Animal Research Committee ethical guidelines at the University of California Los Angeles under protocol no ARC-2015-125. Ten-week-old C56BL/6J mice (Jackson Laboratory) were immunized for a total of 7 times in 28 days with recombinant ovalbumin (Biosearch Technologies) as a model antigen. Immunizations were made by mixing ovalbumin at 1 μg/μL in PBS with equal volume of Inject alum adjuvant (ThermoFisher Scientific). 250 μg and 25 μg doses of ovalbumin were used for initial immunization, and the subsequent 7 boosters, respectively. Four days after the final immunization, mice were euthanized with isoflurane overdose followed by cervical dislocation and sterilized by spraying with 70% ethanol. Isolated cells 50 from bone marrow of up to 4 mice were pooled together, strained through a 70 μm cell strainer, and centrifuged at 300 g for 10 min at 4 C. A 10⁸ cells/mL suspension of cells 50 was prepared in EasySep buffer (StemCell Technologies, #20144), and plasma cells 50 were isolated using a EasySep mouse CD138 positive selection kit (StemCell Technologies, #18957) according to the manufacturer's protocol. The isolated cell suspensions were then placed on ice until use. For B cell/plasmablast isolation, spleen was removed, cut into small pieces with scissors, and pushed through a 70 μm cell strainer in a 10 cm petri dish containing 10 mL cold PBS, using the plunger of a 10 mL syringe. The same syringe was then used to dissociate tissue clumps by drawing and flushing the solution through a 25-gauge needle 3 times. Cells 50 were pelleted by centrifugation at 300 g for 10 min and resuspended at 10⁸ cells/mL in EasySep buffer and B lineage cells were isolated using an EasySep mouse B cell negative isolation kit (StemCell Technologies, #19854). For serum, blood was collected directly from left ventricle via cardiac puncture immediately after euthanasia. Blood was allowed to clot at room temperature for 4 hours, before centrifuging at 10,000 g for 10 min to separate serum.

### B Cell and Plasma Cell Cultures

Plasma and B cells 50 were preferably used fresh after isolation. Excess cells 50 were frozen in FBS containing 10% DMSO at 1 million cells per mL. After thawing, cells 50 were allowed to recover for one hour in media before further use. For some experiments cells 50 were cultured in RPMI media, containing 10% FBS, 1% of each of penicillin/streptomycin, L-Glutamine, HEPES buffer, minimum essential medium nonessential amino acids, sodium pyruvate (ThermoFisher Scientific 15070063, A2916801, 15630130, 11140050, 11360070), and 50 M beta mercapto-ethanol. For any incubation time over 1 hour, the plasma cell media was supplemented with IL-6 and APRIL (GenScript Z03189 and Z02969) at 50 and 100 ng/mL respectively. Isolated B cells 50 from spleen of vaccinated mice were cultured at 500 k cells/mL in vitro for 3 days in media supplemented with LPS and CpG ODN 1826 at 5 and 1 μg/μL, respectively.

Plasma cell secretion assay. To minimize manipulation of primary plasma cells 50, particle functionalization with cell capture antibodies and affinity capture agents 16 was performed prior to seeding the cells 50. To reduce sample loss, as mentioned above, all conical tubes and pipette tips were precoated with blocking or washing buffer prior to handling particle containing solutions. Biotinylated shaped particles 10 were labeled with streptavidin (Thermo Fisher, 434302) by adding 150 ng per μL of concentrated particle solution and incubating for 10 min. Samples were then washed 3× with washing buffer and resuspended. Shaped particles 10 were then labeled with biotinylated anti-CD45 and biotinylated goat anti-mouse IgG H&L chain antibodies (Invitrogen 13045182 and SouthemBiotech 1034-08) by adding 40 ng and 60 ng of the respective antibodies per μL of concentrated particle solution and incubating for 30 min. After incubation, shaped particles 10 were washed and resuspended in EasySep buffer containing IL6 and APRIL, respectively. 9 μL of 55 μm shaped particles 10 and 85,000 plasma cells 50 were seeded into a 24-well plate as described above and then incubated at 37° C. for 1 hour to allow cells 50 to adhere to the shaped particles 10. Shaped particles 10 were then recovered by tilting the well plate and transferring by pipetting. To remove unattached cells 50 from the background, samples were strained using a 37 or 20 μm reversible cell strainer for 55 and 35 μm particles, respectively (STEMCELL Technologies or Partec North America CELLTRICS) and then shaped particles 10 were recovered by flipping the cell strainer and washing with washing buffer. After recovering, samples were concentrated by centrifuging shaped particles 10 and associated cells 50 at 300 g for 3 min, aspirating, and then resuspending in 1 mL EasySep buffer containing IL6 and APRIL. After re-suspending, samples were compartmentalized by pipetting with oil and surfactant as described above to create dropicles. Samples were then incubated for 2 hours to allow cells 50 attached to the shaped particles 10 to secrete and to facilitate capture of secreted antibodies onto the associated particle matrix via goat anti-mouse IgG H&L sites. After the incubation period, the shaped particles 10 and associated cells 50 were transferred back into media by breaking the emulsions. Samples were then washed, and captured secretions were stained with Alexa Fluor 647 conjugated ovalbumin (Invitrogen 034784) at a final concentration of 50 ng per μL of initial concentrated particle solution. After 30 min of staining, samples were washed 3× with a large volume of EasySep buffer and optionally stained with propidium iodide. An aliquot of the sample was then either sorted using a Sony SH800 FACS instrument 100 or imaged with a fluorescence microscope in both brightfield and fluorescence channels in a well plate for quality control, or analysis in MATLAB.

Serum measurements, LOD and Dynamic Range experiments. For assessment of the dynamic range of the on-particle FLISA for antibody capture and detection, 2 μL aliquots of shaped particles 10 were used for each condition. To reduce sample loss, as mentioned above, all conical tubes and pipette tips were precoated with washing buffer prior to handling particle containing solutions. Biotinylated shaped particles 10 were labeled with streptavidin (Thermo Fisher, 434302) by adding 150 ng per μL of concentrated particle solution and incubating for 10 min. Samples were then washed 3× with washing buffer and resuspended. Shaped particles 10 were then functionalized with biotinylated goat anti-mouse IgG H&L antibodies with or without biotinylated anti-CD45 (SouthemBiotech 1034-08, Invitrogen 13045182) by adding 60 ng and 40 ng of the respective antibodies per μL of concentrated particle solution and incubating for 30 min. After incubation, shaped particles 10 were washed and resuspended in washing buffer. Serum, media, or purified antibodies at various concentrations were added to the shaped particles 10 and incubated for 2 hours to allow antibody capture onto the particle matrix via anti-mouse IgG H&L sites. After the incubation period, shaped particles 10 were washed and captured secretions were stained with Alexa Fluor 647 conjugated ovalbumin (Invitrogen 034784) at a final concentration of 50 ng per μL of initial concentrated particle solution. After 30 min of staining, samples were washed 5× with washing buffer. Finally, shaped particles 10 were analyzed by flow cytometry or fluorescence microscopy. A similar assay format was also tested for ovalbumin specific antibody capture using a reverse FLISA setup: biotinylated ovalbumin (Nanocs OVA1BN1) was used as a capture site bound on the shaped particles 10, and Alexa Fluor Plus 555 conjugated goat anti-mouse IgG H&L antibodies (Invitrogen, A32727) for detection, with similar moles of capture and detection molecules as the first FLISA configuration.

Heavy and Light Chain Amplification from Single Cells. Single cells 50 loaded on shaped particles 10 were sorted into 96-well plates containing 5 mM DTT, 0.7% NP-40, μM random hexamers, and 1 U RNasin Plus in PBS and stored at −80° C. until analysis. For first-strand cDNA synthesis, sorted cells 50 were thawed on ice, incubated at 68° C. for 1 min, then incubated on ice for 2 min. Each well then received 10 μL of 2× RT Buffer, 5 mM MgCl₂ and 0.01 M DTT containing 40 U RNasin Plus and 200 U SuperScript III Reverse Transcriptase (Thermo Fisher). The reaction was incubated at 25° C. for 10 min then 50° C. for 50 min. The cDNA product from each single cell 50 was then amplified for both heavy and κ light chains in separate, nested RT-PCR reactions using primers optimized by von Boehmer et al. (von Boehmer, L., Liu, C., Ackerman, S. et al. Sequencing and cloning of antigen-specific antibodies from mouse memory B cells. Nat Protoc 11, 1908-1923 (2016). https://doi.org/10.1038/nprot.2016.102, incorporated herein by reference). All PCR reactions contained 1× HF Buffer, 200 μM dNTPs, and 0.02 U/μL Phusion High Fidelity DNA Polymerase (Thermo Fisher) in a final reaction volume of 20 μL. The initial, preamplification RT-PCRs contained a mixture of forward primers at a final concentration of 0.3 μM, the reverse primer at a concentration of 0.2 μM, and 2 μL of the reverse-transcribed cDNA. The thermocycler conditions included an initial denaturation at 98° C. for 3 min followed by 50 cycles of 10 sec at 98° C., 10 sec at 46° C., and 15 sec at 72° C. then a final extension at 72° C. for 10 min. The second, nested RT-PCRs contained 0.2 μM each of the forward and reverse primers and 2 μL of the preamplification RT-PCR product. The thermocycler conditions were the same as in the preamplification PCR, but with a 57° C. annealing. The second PCR products were visualized on a 2% agarose gel with SYBR Safe DNA gel stain (Thermo Fisher).

Antibody conjugation to shaped particles (multiplexed detection/sorting). Sterile shaped particles 10 were diluted in Washing Buffer five times the volume of shaped particles 10 (i.e., 100 μL of shaped particle volume was resuspended in 400 μL Washing Buffer). Diluted shaped particle suspension was incubated with equal volume of 200 μg/mL of streptavidin for 30 minutes at room temperature on a tube rotator. Excess streptavidin was washed out three times by pelleting shaped particles 10 at 200 g for 5 minutes, removing supernatant and adding 1 mL of fresh Washing Buffer. Streptavidin-coated shaped particles 10 were reconstituted at a five-time dilution in Washing Buffer containing 20 μg/mL of each biotinylated antibody or cocktail of antibodies. anti-CD45 (Biolegend) and anti-IFN-γ (R&D Systems), anti-TNF-α (R&D Systems), or anti-IL-2 (BD Sciences). Shaped particles 10 were incubated with antibodies for 30 minutes at room temperature on a rotator and washed three times as described above. Shaped particles 10 were resuspended at a five times dilution in Washing Buffer or culture medium prior to each experiment.

T cell culture. T cells 50 were isolated from human donor whole blood samples by negative selection using the RosetteSep Human T Cell Enrichment Cocktail Kit (STEMCELL Technologies). Isolated T cells 50 were seeded in a fresh complete ImmunoCult™-XF T cell expansion medium (STEMCELL Technologies) at 1×10⁶ cells/mL with 2 μL/mL ImmunoCult™ human CD3/CD28 T cell activator. T cells 50 were activated for 3 days and expanded for up to 12 days by changing into fresh expansion medium every 2-3 days. All cells 50 were cultured in incubators at 37° C. and 5% CO₂.

Cell loading into shaped particles (multiplexed detection). T cells 50 were stained with 1 μM cell tracker deep red dye (Thermo Fisher) for 30 minutes and loaded into shaped particles 10. Each well of a 24-well plate was filled with 1 mL of T cell expansion medium and 30 μL of reconstituted antibody-labeled shaped particles 10 (6 μL of shaped particle volume=187,000 total shaped particles 10) were added in each well using a standard micropipette. Cells 50 were seeded in each well and extra culture medium was added to make a total volume of 1.5 mL. Each well was mixed by simply pipetting 5 times with a 1000 μL pipette set to 1000 μL. The well plate was transferred to an incubator to allow cell binding for one hour; each well was pipetted up and down again with a 200 μL pipette at 30 minute intervals. After one hour, shaped particles 10 were strained to remove any unbound cells 50 and recovered using a 20 μm cell strainer. During this step, any unbound cells 50 were washed through the strainer and only the shaped particles 10 (with or without cells 50 loaded) were recovered into a conical tube or well plate.

Cell loading efficiency and statistics (multiplexed detection/sorting). Shaped particles 10 labeled with anti-CD45 antibodies were prepared using the procedures described above. To test concentration dependent loading of shaped particles 10 0.15×10⁶ (0.8 cells per shaped particle), 0.3×10⁶ (1.6 cells per shaped particle), and 0.47×10⁶ (2.4 cells per shaped particle 10) of cell tracker deep red stained T cells 50 were seeded in each well of a 24-well plate along with 187,000 shaped particles 10. After incubating for one hour in the incubator to promote cell binding, shaped particles 10 were recovered and transferred to a well plate to be imaged with a fluorescence microscope. Loading efficiency was analyzed using a custom image analysis algorithm in MATLAB which detected shaped particles 10 using standard segmentation algorithms. The software measured the total number of shaped particles 10 in each image frame, then the number of cells 50 in each shaped particle 10 was manually counted to record the total number of shaped particles 10 with 0, 1 or 2 or more cells 50 (n>2000). For comparing different cell binding motifs shaped particles 10 were labeled with 20 μg/mL of each biotinylated antibodies (anti-CD3, anti-CD3 and anti-CD28, or anti-CD45) and seeded with 0.3 million cells 50 in each well. To determine the effect of increased anti-CD45 concentration on shaped particles 10, shaped particles 10 were labeled with 0, 10, 20, or 30 μg/mL of anti-CD45 antibodies and 0.3 million cells 50 were seeded in each well with shaped particles 10 in a 24-well plate. After cell binding and recovery of shaped particles 10, the number of cells 50 in each shaped particle were analyzed using the same image analysis algorithms mentioned above (n>2000).

Dynamic range of cytokine detection on nanovials (multiplexed detection/sorting). Shaped particles 10 were labeled with biotinylated antibodies (20 μg/mL anti-CD45 and 20 μg/mL anti-IFN-γ, anti-TNF-α or anti-IL-2) using the modification steps mentioned above. Each sample of cytokine capture antibody-labeled shaped particles 10 was incubated with 0, 10, 100, or 1000 ng/mL of recombinant human IFN-γ (R&D Systems), TNF-α (R&D Systems), and IL-2 (R&D Systems) for 2 hours at 37° C. Excess proteins were removed by washing shaped particles 10 three times with washing buffer. Shaped particles 10 were pelleted at the last wash step and incubated with 5 μl of 100 μg/mL anti-IFN-γ BV421 (Biolegend), 25 μg/mL anti-TNF-α Alexa 647 (Biolegend) and 50 μg/mL anti-IL-2 Alexa 488 (Biolegend) in 50 μL washing buffer per 6 μL shaped particle volume at 37° C. for 30 minutes protected from light. Following washing three times, shaped particles 10 were reconstituted at a 50 times dilution in the Washing Buffer and transferred to a flow tube. A small fraction of the sample was transferred to a 96-well plate to be imaged on a fluorescence microscope. Fluorescent signal on shaped particles 10 was analyzed using SONY SH800, using violet (405 nm), blue (488 nm), and red (640 nm) lasers with 450/50 nm, 525/50 nm, and 665/30 nm filters.

Analyzing single-cell cytokine secretion on nanovials (multiplexed detection/sorting). Shaped particles 10 were sequentially coated with streptavidin and biotinylated antibodies (20 g/mL anti-CD45 and 20 μg/mL anti-IFN-γ, anti-TNF-α or IL-2). In each well of a 24-well plate, antibody labeled shaped particles 10 and 0.3 million cells 50 were mixed in a total of 1.5 mL T cell expansion medium to allow cell binding. Any unbound cells were removed by a 20 μm cell strainer and shaped particles 10 were recovered into a 12-well plate in 2 mL of T cell expansion medium with 100 ng/mL PMA and 500 ng/mL Ionomycin. T cells 50 were activated for 3 hours in a 37° C. incubator to accumulate secretion and shaped particles 10 were collected in a conical tube with washing buffer. After 5 minutes of centrifugation at 200 g, supernatant was removed and shaped particles 10 were reconstituted at a ten-time dilution in washing buffer containing 5 μl of 100 μg/mL anti-IFN-γ BV421 (Biolegend), 25 μg/mL anti-TNF-α APC (Biolegend) and 200 μg/mL anti-IL-2 APC (BD Biosciences) per 6 μL shaped particle volume. Shaped particles 10 were incubated at 37° C. for 30 minutes, protected from light to label secreted cytokines. After washing shaped particles 10 with 5 mL of washing buffer, shaped particles 10 were resuspended at a 50-time dilution in the washing buffer and transferred to a flow tube, while a small fraction of sample was transferred to 96-well plate to be imaged prior to sorting on a fluorescence microscope. Pre-sort images were analyzed by the custom image analysis algorithms in MATLAB. Fluorescence intensity profile was calculated along a line segment manually defined around the cavity 12 of the shaped particle 10. The intensity peak height and the area under intensity profile were then evaluated, yielding area over height aspect ratio. In FACS, samples were excited using violet (405 nm), blue (488 nm), and red (640 nm) lasers with 450/50 nm, 525/50 nm, and 665/30 nm filters and analyzed based on a combination of fluorescence area and height signals. The population with higher area signal as compared to the other population with similar height values was sorted as shaped particles 10 with secretion signal, while the other population was sorted as shaped particles 10 with intracellular staining. For each cytokine, 40,000 shaped particles 10 were analyzed and directly sorted into a 96-well plate and imaged with a fluorescence microscope to quantify the enrichment of shaped particles 10 with secretion signal or intracellular staining.

Sorting of high, medium, and low secretors. Shaped particles 10 were labeled with streptavidin and biotinylated antibodies (20 μg/mL anti-CD45 and 20 μg/mL anti-IFN-γ or anti-TNF-α). Antibody-labeled shaped particles 10 (187,000) and 0.3 million cells 50 were mixed in a total of 1.5 mL T cell expansion medium to allow cell binding for one hour in a 24-well plate. Shaped particles 10 were recovered using a 20 μm cell strainer and seeded into a 12-well plate in 2 mL of T cell expansion medium with 100 ng/mL PMA and 500 ng/mL ionomycin. T cells 50 were activated for 3 hours in a 37° C. incubator to accumulate secretion and shaped particles 10 were reconstituted at a ten times dilution in Washing Buffer containing 0.3 μM calcein AM dye (Thermo Fisher) along with 5 μL of each detection antibody (anti-IFN-γ BV421, anti-TNF-α APC, anti-IL-2 APC) per 6 μL shaped particle volume. Shaped particles 10 were incubated at 37° C. for 30 minutes, protected from light. Shaped particles 10 were resuspended at a 50 times dilution in the Washing Buffer and transferred to a flow tube to be analyzed by FACS. To sort live single cells 50 based on secretion signal, shaped particles 10 with calcein staining were first gated and high, medium, low secretors were sorted by thresholding the fluorescence area and height signals as shown in the results. Sorted samples were imaged with a fluorescence microscope to validate the enrichment of shaped particles 10 based on the amount of secretion captured on the shaped particles 10.

Linking cell surface markers to secretion phenotype. Analysis of secretions across multiple cytokines was performed and linked this to surface markers. Streptavidin-coated shaped particles 10 were decorated with biotinylated antibodies (20 μg/mL anti-CD45, 20 μg/mL anti-IFN-γ, 20 μg/mL anti-TNF-α or 20 μg/mL anti-CD45, 20 μg/mL anti-IFN-γ, 20 μg/mL IL-2) for 30 minutes. Negative control shaped particles 10 were also prepared by labeling shaped particles 10 only with anti-CD45 antibody without any cytokine capture antibodies. In each well of a 24-well plate, shaped particles 10 were seeded with 0.5 million cells 50 in a total of 1.5 mL T cell expansion medium. Cells 50 were allowed to bind for 1.5 hour in a 37° C. incubator. After straining and recovery of shaped particles 10, cells 50 were activated for 3 hours in the T cell expansion medium containing 100 ng/mL PMA and 500 ng/mL ionomycin. To stain secreted cytokines, shaped particles 10 were washed once with 5 mL Washing Buffer and reconstituted at a ten times dilution in Washing Buffer containing 5 μL of each detection antibody (anti-IFN-γ BV421, anti-TNF-α APC, anti-IL-2 APC), 5 μL of 25 μg/mL anti-CD4 PE (Biolegend) and 5 μL of 100 μg/mL anti-CD8 AlexaFluor 488 (Biolegend) per 6 μL shaped particle volume for 30 minutes in a 37° C. incubator. Shaped particles 10 were resuspended at a 50 times dilution in the Washing Buffer and transferred to a flow tube to be analyzed in FACS. In flow, shaped particles 10 with CD4 and CD8 cells 50 were gated based on fluorescence area signals of AlexaFluor 488 and PE. Shaped particles 10 with secretion signal were evaluated by first creating quadrant gates based on the negative control sample (shaped particles 10 only labeled with anti-CD45 antibody). Q1 was defined as shaped particles 10 with only IFN-γ secreting cells 50. Q2 was shaped particles 10 with polyfunctional T cells 50 that secreted both cytokines (IFN-γ and TNF-α or IL-2). Q3 was shaped particles 10 with either TNF-α or IL-2 secreting cells 50 while Q4 was shaped particles 10 with non-secretors. Shaped particles 10 in each quadrant were sorted into a 96-well plate and samples were imaged with a fluorescence microscope to quantify enrichment of each cell type and their associated secretion characteristics. For each multiplexed screening, 100,000 shaped particles 10 were analyzed and sorted by FACS per condition and each condition was repeated three times for a total of 300,000 shaped particles 10 analyzed per condition.

While embodiments of the present invention have been shown and described, various modifications may be made without departing from the scope of the present invention. The invention, therefore, should not be limited, except to the following claims, and their equivalents.

1. A shaped particle system comprising:
a plurality of three-dimensional shaped particles, each shaped particle having a void or cavity formed therein that comprises a single opening to an external environment of the shaped particle, wherein each shaped particle further comprises a poly-L-lysine (PLL)-containing surface in the void or cavity.

2. The shaped particle system of claim 1, further comprising an extracellular matrix protein or protein fragment disposed on the surface of the void or cavity.

3. The shaped particle system of claim 1, wherein the extracellular matrix protein comprises fibronectin or retronectin.

4. The shaped particle system of claim 1, further comprising a cell adhered to the shaped particle within the void or cavity.

5. The shaped particle system of claim 1, wherein a longest dimensional length of the shaped particles is <100 micrometers.

6. (canceled)

7. A shaped particle system comprising:
a plurality of three-dimensional shaped particles, each shaped particle having a void or cavity formed therein that comprises a single opening to an external environment of the shaped particle, wherein each shaped particle further comprises a cell binding moiety and one or more capture agents disposed on a surface of the shaped particle in the void or cavity;
wherein the cell binding moiety comprise one or more of: (1) anti-CD45 antibodies or a fragment thereof, anti-CD19 antibodies or a fragment thereof, anti-CD3 antibodies or a fragment thereof, anti-CD28 antibodies or a fragment thereof, streptavidin, biotin, an antigen, a peptide-major histocompatibility complex; and the one or more capture agents comprise one or more of: (2) anti-IL-2, anti-TNF-α, anti-IFN-γ antibodies or fragments thereof.

8. (canceled)

9. The shaped particle system of claim 7, further comprising a cell adhered to the surface of the shaped particle within the void or cavity and wherein the cell comprises a B cell, plasmablast, plasma cell, T cell, NK cell, or CHO cell.

10. (canceled)

11. The shaped particle system of claim 7, wherein a longest dimensional length of the shaped particle is <100 micrometers.

12. (canceled)

13. A method of screening B or T cells for one or more secretions of interest using a plurality of three-dimensional shaped particles comprising:
loading single B or T cells into respective voids or cavities formed in the plurality of three-dimensional shaped particles;
capturing the one or more secretions of interest with one or more capture agents disposed on or in the plurality of three-dimensional shaped particles;
labeling the one or more captured secretions of interest with fluorescent reporters;
sorting the three-dimensional shaped particles using a flow cytometer or fluorescence activated cell sorter based on a fluorescence signal from the fluorescent reporters to create a sorted population of three-dimensional shaped particles.

14. The method of claim 13, further comprising
performing nucleic acid sequence analysis on nucleic acids contained in at least some of the sorted population of three-dimensional shaped particles.

15. The method of claim 14, wherein the B or T cells are exposed to a lysing agent and subject to nucleic acid amplification prior to performing nucleic acid sequence analysis.

16. The method of claim 13, wherein a plurality of capture agents are disposed on or in the plurality of three-dimensional shaped particles, wherein each of the plurality of capture agents are specific to different secretions of interest.

17. The method of claim 13, further comprising labeling one or more cell surface markers with fluorescent reporters.

18. The method of claim 13, wherein the T cells comprise chimeric antigen receptor (CAR)-T cells.

19. The method of claim 13, wherein the secretion of interest comprises a cytokine.

20. The method of claim 13, wherein the capture agent comprises protein A, protein G, anti-IgG antibody, anti-Fc antibody, anti-H&L antibody or a fragment thereof.

21. The method of claim 20, wherein the fluorescent reporter comprises a fluorescently-labelled antigen.

22. The method of claim 13, wherein the capture agent comprises an antigen.

23. The method of claim 22, wherein the fluorescent reporter comprises a fluorescently-labelled anti-IgG antibody, anti-Fc antibody, anti-H&L antibody or a fragment thereof.

24. The method of claim 13, wherein the sorted population of three-dimensional shaped particles are sorted into respective vessels, wells, droplets, or containers each containing a single shaped particle.

25. The method of claim 13, wherein a single three-dimensional shaped particle from the sorted population of three-dimensional shaped particles is sorted to a specific well in a multiwell plate.

26. The method of claim 25, wherein the fluorescence signal associated with the single three-dimensional shaped particle from the sorted population is linked in a data record that maps to the location of the specific well in the multiwell plate.

27. The method of claim 26, wherein the nucleic acid sequence analysis is linked in the data record to the fluorescence signal associated with the single three-dimensional shaped particle.

28. The method of claim 13, wherein the fluorescence signal comprises a fluorescence intensity peak that is defined by a fluorescence height, fluorescence width, and fluorescence area.

29. The method of claim 28, wherein sorting is based on thresholds or gates on two or more of: fluorescence height, fluorescence width, and fluorescence area.

30. A method of sorting shaped particles loaded with cells using a flow cytometer or FACS instrument comprising:
providing a population of shaped particles loaded with single cells into respective voids or cavities formed in the plurality of three-dimensional shaped particles;
capturing a secretion from the cells on one or more of the shaped particles containing single cells therein;
exposing the one or more shaped particles with the captured secretion to a fluorescent reporter;
flowing the population of shaped particles loaded with single cells through the flow cytometer or FACS instrument;
optically interrogating the shaped particles in the flow cytometer or FACS instrument to measure a fluorescence signal for each shaped particle that comprises two or more of: a fluorescence peak area, fluorescence peak height, and fluorescence peak width; and
identifying and sorting a sub-population of the shaped particles based at least in part on a threshold or gate on two or more of: fluorescence peak area, fluorescence peak height, and fluorescence peak width.

31. The method of claim 30, wherein the number of shaped particles in the population of shaped particles is greater than 100,000.

32. The method of claim 30, wherein the sub-population is identified and sorted based on a ratio between fluorescence peak area and fluorescence peak height.

33. The method of claim 32, wherein the sub-population is identified and sorted based on a threshold ratio, wherein a sub-population having a ratio above the threshold ratio are sorted separately from another sub-population having a ratio below the threshold ratio.

34. The method of claim 30, wherein the cells comprise B cells or T cells.
